(19) United States
(12) Patent Application Publication
(10) Pub. No.: US 2011/0263593 A1
(43) Pub. Date: Oct. 27, 2011

(54) NOVEL IMIDAZO[1,2-a]PYRIMIDINE DERIVATIVES, METHOD FOR THE PREPARATION THEREOF, USE THEREOF AS MEDICAMENTS, PHARMACEUTICAL COMPOSITIONS AND NOVEL USE, IN PARTICULAR AS MET INHIBITORS

(75) Inventors: Eric Bacque, Gif Sur Yvette (FR); Dominique Damour, Orly (FR); Conception Nemecek, Orveau (FR); Patrick Nemecek, Orveau (FR); Laurent Schio, Marolles en Brie (FR); Sylvie Wentzler, L'Hay les Roses (FR)

(73) Assignee: sanofi-aventis, Paris (FR)

(21) Appl. No.: 13/007,830

(22) Filed: Jan. 17, 2011

Related U.S. Application Data
(63) Continuation of application No. PCT/FR2009/051408, filed on Jul. 16, 2009.

(30) Foreign Application Priority Data
Jul. 18, 2008 (FR) 0804086

(51) Int. Cl. A61K 31/519; C07D 417/10; A61K 31/5377; A61P 9/00; A61P 7/04; A61P 3/00; A61P 37/08; A61P 11/06; A61P 7/02; A61P 25/00; A61P 9/10; A61P 17/06; A61P 29/00; A61P 3/10; A61P 21/00; A61P 35/00; C07D 471/04

(52) U.S. Cl. 514/233.2; 544/281; 514/259.1; 544/135; 514/233.8; 544/117

(57) ABSTRACT

The disclosure concerns imidazo[1,2-a]pyrimidine derivatives of formula (I) and isomers and pharmaceutically acceptable salts thereof. Methods for preparing the compounds, pharmaceutical compositions, and methods of treatment also are disclosed.

### CROSS-REFERENCE TO RELATED APPLICATIONS

This application is a continuation of International application No. PCT/FR2009/051408, filed Jul. 16, 2009, which claims the benefit of priority of French Patent Application No. 0804086, filed Jul. 18, 2008, both of which are incorporated herein by reference.

The present invention relates to novel imidazo[1,2-a]pyrimidine derivatives, to their process of preparation, to the novel intermediates obtained, to their application as medicaments, to the pharmaceutical compositions including them and to the novel use of such imidazo[1,2-a]pyrimidine derivatives.

The present invention relates more particularly to novel imidazo[1,2-a]pyrimidine derivatives exhibiting an anticancer activity via the regulation of the activity of proteins, in particular kinases.

Currently, most commercial compounds used in chemotherapy are cytotoxic compounds which present significant problems of side effects and of tolerance by the patients. These effects might be limited in so far as the medicaments used act selectively on cancerous cells to the exclusion of healthy cells. One of the solutions for limiting the undesirable effects of a chemotherapy can thus consist of the use of medicaments which act on metabolic pathways or constituent components of these pathways which are predominantly expressed in cancerous cells and which would not be expressed or which would be only slightly expressed in healthy cells. Protein kinases are a family of enzymes which catalyse the phosphorylation of hydroxyl groups of specific protein residues, such as tyrosine, serine or threonine. Such phosphorylations can extensively modify the function of the proteins; thus, protein kinases play a major role in the regulation of a wide variety of cell processes, including in particular metabolism, cell proliferation, cell adhesion and motility, cell differentiation or cell survival, some protein kinases playing a central role in initiation, development and completion of the events of the cell cycle.

The various cell functions in which activity of a protein kinase is involved include some processes which represent attractive targets for treating certain diseases. Mention may in particular be made, as example, of angiogenesis and the control of the cell cycle and also that of cell proliferation, in which protein kinases can play an essential role. These processes are in particular essential for the growth of solid tumours and of other diseases; in particular, molecules which inhibit such kinases are capable of limiting undesirable cell proliferation, such as that observed in cancers, and can intervene in the prevention, regulation or treatment of neurodegenerative diseases, such as Alzheimer's disease or neuronal apoptosis.

The subject-matter of the present invention is novel derivatives having inhibitory effects with regard to protein kinases. The products according to the present invention can thus in particular be used for the prevention or treatment of diseases which can be regulated by the inhibition of protein kinases.

The products according to the present invention exhibit in particular an anticancer activity via the regulation of the activity of kinases. Preference is given, among the kinases for which a regulation of the activity is desired, to MET and to mutants of the MET protein.

The present invention also relates to the use of the said derivatives in the preparation of a medicament intended for the treatment of man.

Thus, one of the objects of the present invention is to provide compositions having anticancer activity by acting in particular with regard to kinases. Preference is given, among the kinases for which a regulation of the activity is desired, to MET.

In the pharmacological part below, it is shown, in biochemical tests and with regard to cell lines, that the products of the present patent application thus inhibit in particular the autophosphorylation activity of MET and the proliferation of the cells whose growth depends on MET or on its mutant forms.

MET, or Hepatocyte Growth Factor Receptor, is a receptor having tyrosine kinase activity expressed in particular by epithelial and endothelial cells. HGF, Hepatocyte Growth Factor, is described as the specific ligand for MET. HGF is secreted by mesenchymal cells and activates the MET receptor, which homodimerizes. Consequently, the receptor autophosphorylates on the tyrosines of the catalytic domain Y1230, Y1234 and Y1235.

The stimulation of MET by HGF induces cell proliferation, scattering (or dispersion) and motility, resistance to apoptosis, invasion and angiogenesis.

MET as well as HGF are found to be overexpressed in many human tumours and a wide variety of cancers. MET is also found to be amplified in gastric tumours and glioblastomas. Many point mutations of the MET gene have also been described in tumours, in particular in the kinase domain, but also in the juxtamembrane domain and the SEMA domain. Overexpression, amplification or mutations cause constitutive activation of the receptor and deregulation of its functions.

The present invention thus relates in particular to novel inhibitors of the MET protein kinase and of its mutants which can be used in an antiproliferative and antimetastatic treatment, in particular in oncology.

The present invention also relates to novel inhibitors of the MET protein kinase and of its mutants which can be used in an antiangiogenic treatment, in particular in oncology.

A subject-matter of the present invention is the products of formula (I):

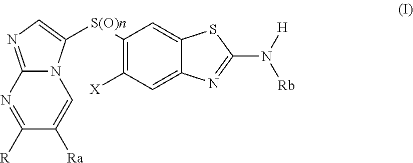

in which:

n=0, 1 or 2;

X represents a hydrogen atom, a halogen atom or an alkyl radical;

R represents a hydrogen atom or an NH₂, NHalk or N(alk)₂ radical;

Ra represents a hydrogen atom, a halogen atom or an —O-cycloalkyl, —O-alkyl, —O-aryl, —O-heteroaryl, —NRd(cycloalkyl), —NRd(alkyl), —NRd(aryl), —NRd(heteroaryl), alkyl, cycloalkyl, heterocycloalkyl, aryl or heteroaryl radical; the cycloalkyl, alkyl, aryl and heteroaryl radicals being, in all these radicals, optionally substituted as indicated below;

Rb represents a hydrogen atom or an Rc, —COORc, —CO—Rc or —CO—NRcRd radical;

with Rc representing an alkyl, cycloalkyl, heterocycloalkyl, aryl or heteroaryl radical, all these radicals being optionally substituted as indicated below;

Rd represents a hydrogen atom or an alkyl or cycloalkyl radical;

all the radicals defined above, alkyl, cycloalkyl, heterocycloalkyl, aryl and heteroaryl, being optionally substituted by one or more radicals chosen from halogen atoms and hydroxyl, alkoxyl, CN, CF₃, —NR1R2, —COOH, —COOalk, —CONR1R2, —NR1COR2, COR1, oxo, heterocycloalkyl, aryl and heteroaryl radicals, the latter heterocycloalkyl, aryl or heteroaryl being themselves optionally substituted by one or more radicals chosen from halogen atoms and hydroxyl, alkoxy, alkyl, CN, CF₃, —NR3R4, —COOH, —COOalk, —CONR3R4, —NR3COR4, —COR3 and oxo radicals;

the cycloalkyl, heterocycloalkyl, aryl or heteroaryl radicals being in addition optionally substituted by an alkyl radical itself optionally substituted by one or more radicals chosen from halogen atoms and hydroxyl, alkoxy, alkyl, NR3R4, —COOH, —COOalk, —CONR3R4, —NR3COR4 and —COR3 radicals;

NR1R2 being such that: either, R1 and R2 being identical or different, one of R1 and R2 represents a hydrogen atom or an alkyl radical and the other of R1 and R2 represents a hydrogen atom, a —CO₂-alkyl radical, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 3 to 10 ring members and optionally one or more other heteroatoms chosen from O, S and NH, this radical, including the optional NH which it comprises, being optionally substituted;

NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R3 and R4 form, with the nitrogen atom to which they are bonded, a cyclic radical including from 3 to 10 ring members and optionally one or more other heteroatoms chosen from O, S and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals chosen from halogen atoms, hydroxyl, oxo, alkoxy, NH₂, NHalk and N(alk)₂ radicals and alkyl, cycloalkyl, heterocycloalkyl, —CO-alkyl, —CO₂Alk, phenyl, CH₂-phenyl and heteroaryl radicals, such that, in the latter radicals, the alkyl, heterocycloalkyl, phenyl and heteroaryl radicals are themselves optionally substituted by one or more radicals chosen from halogen atoms, hydroxyl radicals, alkyl and alkoxy radicals including from 1 to 4 carbon atoms, and NH₂, NHalk and N(alk)₂ radicals;

R1, R2, R3 and R4 in the —NR1COR2, —COR1, —NR3COR4 and —COR3 radicals being chosen from the meanings indicated above for R1, R2, R3 and R4 in NR1R2 and NR3R4 when R1 and R2, on the one hand, and R3 and R4 do not form a cyclic radical with the nitrogen atom to which they are bonded;

all the alkyl(alk) and alkoxy radicals above including from 1 to 6 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which R and Ra both represent a hydrogen atom, n represents the integer 0, 1 or 2 and the X and Rb substituents are chosen from the meanings indicated above or below for these X and Rb substituents,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which R represents a hydrogen atom, n represents the integer 0, 1 or 2 and the X, Ra and Rb substituents are chosen from the meanings indicated above or below for these X, Ra and Rb substituents, Ra not representing a hydrogen atom,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which Ra represents a hydrogen atom, n represents the integer 0, 1 or 2 and the X, R and Rb substituents are chosen from the meanings indicated above or below for these X, R and Rb substituents, R not representing a hydrogen atom,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which X represents a hydrogen atom, n represents the integer 0, 1 or 2 and the R, Ra and Rb substituents are chosen from the meanings indicated above or below for these R, Ra and Rb substituents,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which X represents a fluorine atom, n represents the integer 0, 1 or 2 and the R, Ra and Rb substituents are chosen from the meanings indicated above or below for these R, Ra and Rb substituents,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

n=0, 1 or 2;

X represents a hydrogen atom, a fluorine atom or a methyl radical;

R represents a hydrogen atom or an NH₂ radical;

Ra represents a hydrogen atom, a halogen atom or an —O-cycloalkyl, —O-alkyl, —NRd(cycloalkyl), —NRd(alkyl), aryl or heteroaryl radical; in all these radicals, the cycloalkyl, alkyl, aryl and heteroaryl radicals being optionally substituted as indicated below;

Rb represents a hydrogen atom, a —CO—Rc radical or a —CO—NRcRd radical;

with Rc representing an alkyl, cycloalkyl, heterocycloalkyl or aryl radical, all optionally substituted by one or more radicals chosen from halogen atoms, hydroxyl, alkoxy and NR1R2 radicals and alkyl, heterocycloalkyl, aryl and heteroaryl radicals, themselves optionally substituted as indicated below;

Rd represents a hydrogen atom or an alkyl radical;

all the radicals defined above, alkyl, cycloalkyl, heterocycloalkyl, aryl and heteroaryl, being optionally substituted by one or more radicals chosen from halogen atoms and hydroxyl, alkoxy, —NR1R2, —COOH, —COOalk, —CONR1R2, alkyl and heterocycloalkyl radicals, itself optionally substituted by one or more radicals chosen from halogen atoms and hydroxyl, alkoxy, alkyl, —COOH, —COOalk, —NR3R4 and —CONR3R4 radicals;

NR1R2 being such that: either, R1 and R2 being identical or different, one of R1 and R2 represents a hydrogen atom or an alkyl radical and the other of R1 and R2 represents a hydrogen atom, a —CO₂-alk radical, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 3 to 10 ring members and optionally one or more other heteroatoms chosen from O, S and NH, this radical, including the optional NH which it comprises, being optionally substituted;

NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R3 and R4 form, with the nitrogen atom to which they are bonded, a cyclic radical including from 3 to 10 ring members and optionally one or more other heteroatoms chosen from O, S and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals chosen from halogen atoms, hydroxyl, oxo, alkoxy, NH₂, NHalk or N(alk)₂ radicals and alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and hydroxyl, alkyl, alkoxy, NH₂, NHalk and N(alk)₂ radicals;

all the alkyl(alk) or alkoxy radicals above including from 1 to 6 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

n=0, 1 or 2;

X represents a hydrogen atom or a fluorine atom;

R represents a hydrogen atom or an NH₂ radical;

Ra represents a hydrogen atom, a halogen atom, an —O-cycloalkyl radical, an —NH-cycloalkyl radical, an —NH-alk-phenyl radical or a phenyl radical, all these cycloalkyl and phenyl radicals being optionally substituted by one or more radicals chosen from halogen atoms and hydroxyl, alkoxy, —NR1R2, —COOH, —COOalk, —CONR1R2, alkyl and heterocycloalkyl radicals, themselves optionally substituted by one or more radicals chosen from halogen atoms and alkyl, —COOH, —COOalk and —CONR3R4 radicals;

Rb represents a hydrogen atom, a —CO—Rc radical or a —CO—NRcRd radical;

with Rc representing an alkyl, cycloalkyl, heterocycloalkyl or aryl radical, all optionally substituted by one or more radicals chosen from hydroxyl, alkoxy, NR1R2, alkyl, heterocycloalkyl and phenyl radicals, the latter alkyl, heterocycloalkyl and phenyl radicals being themselves optionally substituted by one or more radicals chosen from halogen atoms and hydroxyl, alkoxy, alkyl and NR3R4 radicals;

Rd represents a hydrogen atom or an alkyl radical;

NR1R2 being such that either, R1 and R2 being identical or different, one of R1 and R2 represents a hydrogen atom or an alkyl radical and the other of R1 and R2 represents a hydrogen atom, a cycloalkyl radical, a CO₂alk radical or an alkyl radical optionally substituted by one or more identical or different radicals chosen from hydroxyl, alkoxy, NR3R4 or phenyl radicals, itself optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 4 to 7 ring members and optionally another heteroatom chosen from O, S and NH, this radical, including the optional NH which it comprises, being optionally substituted;

NR3R4 being such that either R3 and R4, which are identical or different, represent a hydrogen atom or an alkyl radical optionally substituted by one or more identical or different radicals chosen from hydroxyl or alkoxy radicals or R3 and R4 form, with the nitrogen atom to which they are bonded, a cyclic radical including from 4 to 7 ring members and optionally another heteroatom chosen from O, S and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals as defined in either one of claims 1 and 2;

all the alkyl(alk) or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

n=0, 1 or 2;

X represents a hydrogen atom or a fluorine atom;

R represents a hydrogen atom or an NH₂ radical;

Ra represents a hydrogen atom, a halogen atom, an —O-cycloalkyl radical, an —NH-cycloalkyl radical, an —NH-alk-phenyl radical or a phenyl radical, the phenyl radicals being optionally substituted by one or more radicals chosen from halogen atoms and the alkyl radical;

Rb represents a hydrogen atom, a —CO—Rc radical or a —CO—NRcRd radical;

with Rc representing an alkyl, cycloalkyl, heterocycloalkyl or phenyl radical, all optionally substituted by one or more radicals chosen from hydroxyl, alkoxy, NR1R2, alkyl and heterocycloalkyl radicals, the latter alkyl and heterocycloalkyl radicals being themselves optionally substituted by one or more radicals chosen from halogen atoms and the hydroxyl, alkoxy, alkyl and NR3R4 radicals;

Rd represents a hydrogen atom;

NR1R2 being such that either R1 and R2, which are identical or different, represent a hydrogen atom or an alkyl radical optionally substituted by one or more identical or different radicals chosen from hydroxyl, alkoxy, NH₂, NHalk and N(alk)₂ radicals or else NR1R2 represents the —NHCO₂alk radical; or R1 and R2 form, with the nitrogen atom to which they are bonded, a cyclic radical including from 4 to 7 ring members and optionally another heteroatom chosen from O, S and NH, optionally substituted by one or more identical or different radicals chosen from oxo, NH₂, NHalk, N(alk)₂, alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and alkyl, hydroxyl, alkoxy, NH₂, NHalk and N(alk)₂ radicals;

NR3R4 being such that either R3 and R4, which are identical or different, represent a hydrogen atom or an alkyl radical optionally substituted by one or more identical or different radicals chosen from hydroxyl or alkoxy radicals or R3 and R4 form, with the nitrogen atom to which they are bonded, a cyclic radical including from 4 to 7 ring members and optionally another heteroatom chosen from O, S and NH, this radical, including the optional NH which it comprises, being optionally substituted by an alkyl or phenyl radical, themselves optionally substituted by one or more identical or different radicals chosen from halogen atoms and alkyl, hydroxyl, alkoxy, NH₂, NHalk and N(alk)₂ radicals;

all the alkyl(alk) or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention relates in particular to the products of formula (I) in which X and R both represent a hydrogen atom, n represents the integer 0 and the Ra and Rb substituents are chosen from the meanings indicated above or below for these X and Rb substituents,

the said products of formula (I) being in all the isomeric forms possible, racemic, enantiomeric and diastereoisomeric, and also the addition salts with inorganic and organic acids or with inorganic and organic bases of the said products of formula (I).

The present invention thus relates to the products of following formula (I):

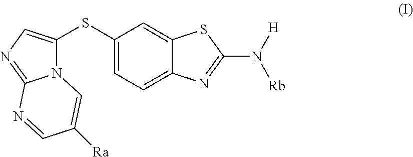

in which Ra and Rb are chosen from the meanings indicated above or below,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) in which:

Ra represents a hydrogen atom, a halogen atom, an aryl radical or a heteroaryl radical, these aryl and heteroaryl radicals being optionally substituted as indicated below;

Rb represents a hydrogen atom or an Rc, —COORc, —CO—Rc or —CO—NRcRd radical;

with Rc representing an alkyl, cycloalkyl, heterocycloalkyl, aryl and heteroaryl radical, all these radicals being optionally substituted as indicated below;

Rd represents a hydrogen atom or an alkyl or cycloalkyl radical;

all the radicals defined above, alkyl, cycloalkyl, heterocycloalkyl, aryl and heteroaryl, being optionally substituted by one or more radicals chosen from halogen atoms and hydroxyl, alkoxy, CN, CF₃, —NR1R2, —COOH, —COOalk, —CONR1R2 and —NR1COR2 radicals;

the alkyl radicals being in addition optionally substituted by an aryl or heteroaryl radical, themselves optionally substituted by one or more radicals chosen from halogen atoms and hydroxyl, alkyl, alkoxy and NR3R4 radicals;

the cycloalkyl, heterocycloalkyl, aryl or heteroaryl radicals being in addition optionally substituted by an alkyl radical, itself optionally substituted by one or more radicals chosen from halogen atoms and hydroxyl, alkyl, alkoxy and NR3R4 radicals;

NR1R2 being such that: either, R1 and R2 being identical or different, one of R1 and R2 represents a hydrogen atom or an alkyl radical and the other of R1 and R2 represents a hydrogen atom, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted; or R1 and R2 form, with the nitrogen atom to which they are bonded, a cyclic radical including from 3 to 10 ring members and optionally one or more other heteroatoms chosen from O, S, N and NH, this radical, including the optional NH which it comprises, being optionally substituted;

NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted; or R3 and R4 form, with the nitrogen atom to which they are bonded, a cyclic radical including from 3 to 10 ring members and optionally one or more other heteroatoms chosen from O, S, N and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals chosen from halogen atoms, hydroxyl, oxo, alkoxy, NH₂, NHalk and N(alk)₂ radicals and alkyl, phenyl, CH₂-phenyl and heteroaryl radicals, such that, in the latter radicals, the alkyl, phenyl and heteroaryl radicals are themselves optionally substituted by one or more radicals chosen from halogen atoms, hydroxyl radicals, alkyl and alkoxy radicals including from 1 to 4 carbon atoms, and NH₂, NHalk and N(alk)₂ radicals;

all the alkyl(alk) and alkoxy radicals above including from 1 to 6 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

Ra represents a hydrogen atom, a halogen atom or an aryl or heteroaryl radical, these aryl and heteroaryl radicals being optionally substituted as indicated below;

Rb represents a hydrogen atom, a —CO—Rc radical or a —CO—NRcRd radical;

with Rc representing an alkyl radical or a cycloalkyl radical, both optionally substituted by one or more radicals chosen from hydroxyl, alkoxy, NR1R2, heterocycloalkyl, aryl and heteroaryl radicals, themselves optionally substituted as indicated below;

Rd represents a hydrogen atom or an alkyl radical;

all the radicals defined above, alkyl, cycloalkyl, heterocycloalkyl, aryl and heteroaryl, being optionally substituted by one or more radicals chosen from halogen atoms and hydroxyl, alkoxy, —NR1R2, —COOH, —COOalk and —CONR1R2 radicals;

NR1R2 being such that: either, R1 and R2 being identical or different, one of R1 and R2 represents a hydrogen atom or an alkyl radical and the other of R1 and R2 represents a hydrogen atom, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted; or R1 and R2 form, with the nitrogen atom to which they are bonded, a cyclic radical including from 3 to 10 ring members and optionally one or more other heteroatoms chosen from O, S, N and NH, this radical, including the optional NH which it comprises, being optionally substituted;

NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted; or R3 and R4 form, with the nitrogen atom to which they are bonded, a cyclic radical including from 3 to 10 ring members and optionally one or more other heteroatoms chosen from O, S, N and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals chosen from halogen atoms, hydroxyl and alkoxy radicals and alkyl, phenyl and CH₂-phenyl radicals, in which the alkyl or phenyl radicals are themselves optionally substituted by one or more identical or different radicals chosen from halogen atoms and alkyl, hydroxyl, alkoxy, NH₂, NHalk and N(alk)₂ radicals;

all the alkyl(alk) or alkoxy radicals above including from 1 to 6 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

Ra represents a hydrogen atom, a halogen atom or an optionally substituted phenyl radical as indicated below;

Rb represents a hydrogen atom, a —CO—Rc radical or a —CO—NRcRd radical;

with Rc representing an alkyl or cycloalkyl radical, both optionally substituted by one or more radicals chosen from hydroxyl, alkoxy, NR1R2 and phenyl radicals, itself optionally substituted by one or more radicals chosen from halogen atoms and hydroxyl, alkoxy, alkyl, NH₂, NHalk and N(alk)₂ radicals;

Rd represents a hydrogen atom or an alkyl radical;

NR1R2 is such that: either, R1 and R2 being identical or different, one of R1 and R2 represents a hydrogen atom or an alkyl radical and the other of R1 and R2 represents a hydrogen atom, a cycloalkyl radical or an alkyl radical optionally substituted by one or more identical or different radicals chosen from hydroxyl, alkoxy, NR3R4 or phenyl radicals, themselves optionally substituted; or R1 and R2 form, with the nitrogen atom to which they are bonded, a cyclic radical including from 4 to 7 ring members and optionally another heteroatom chosen from O, S, N and NH, this radical, including the optional NH which it comprises, being optionally substituted;

NR3R4 being such that either R3 and R4, which are identical or different, represent a hydrogen atom or an alkyl radical optionally substituted by one or more identical or different radicals chosen from hydroxyl or alkoxy radicals or R3 and R4 form, with the nitrogen atom to which they are bonded, a cyclic radical including from 4 to 7 ring members and optionally another heteroatom chosen from O, S, N and NH, this radical, including the optional NH which it comprises, being optionally substituted;

the cyclic radicals which R1 and R2 or R3 and R4 respectively can form with the nitrogen atom to which they are bonded being optionally substituted by one or more identical or different radicals as defined in either one of claims 1 and 2;

all the alkyl(alk) or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

Ra represents a hydrogen atom, a halogen atom or a phenyl radical optionally substituted by a halogen atom;

Rb represents a hydrogen atom, a —CO—Rc radical or a —CO—NRcRd radical;

with Rc representing an alkyl or cycloalkyl radical which is optionally substituted by one or more radicals chosen from hydroxyl, alkoxy and NR1R2 radicals;

Rd represents a hydrogen atom;

NR1R2 being such that either R1 and R2, which are identical or different, represent a hydrogen atom or an alkyl radical optionally substituted by one or more identical or different radicals chosen from hydroxyl, alkoxy, NH₂, NHalk and N(alk)₂ radicals or R1 and R2 form, with the nitrogen atom to which they are bonded, a cyclic radical including from 4 to 7 ring members and optionally another heteroatom chosen from O, S, N and NH, optionally substituted by an alkyl, phenyl or —CH₂-phenyl radical, the latter radicals being themselves optionally substituted by one or more identical or different radicals chosen from halogen atoms and alkyl, hydroxyl, alkoxy, NH₂, NHalk and N(alk)₂ radicals;

all the alkyl(alk) or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

In the products of formula (I) and in that which follows:
the term alkyl (or alk) radical denotes the linear and, if appropriate, branched radicals methyl, ethyl, propyl, isopropyl, butyl, isobutyl, sec-butyl, tert-butyl, pentyl, isopentyl, hexyl or isohexyl and also heptyl, octyl, nonyl and decyl, and also their linear or branched positional isomers; preference is given to the alkyl radicals including from 1 to 6 carbon atoms and more particularly to the alkyl radicals including from 1 to 4 carbon atoms of the above list;
the term alkoxy radical denotes the linear and, if appropriate, branched radicals methoxy, ethoxy, propoxy, isopropoxy, linear, secondary or tertiary butoxy, pentoxy or hexoxy, and also their linear or branched positional isomers; preference is given to the alkoxy radicals including from 1 to 4 carbon atoms of the above list;
the term halogen atom denotes the chlorine, bromine, iodine or fluorine atoms and preferably the chlorine, bromine or fluorine atom;
the term cycloalkyl radical denotes a saturated carbocyclic radical including from 3 to 10 carbon atoms and thus denotes in particular the cyclopropyl, cyclobutyl, cyclopentyl and cyclohexyl radicals and very particularly the cyclopropyl, cyclopentyl and cyclohexyl radicals;
the term heterocycloalkyl radical thus denotes a monocyclic or bicyclic carbocyclic radical including from 3 to 10 ring members which is interrupted by one or more identical or different heteroatoms chosen from oxygen, nitrogen or sulphur atoms; mention may be made, for example, of the morpholinyl, thiomorpholinyl, homomorpholinyl, aziridyl, azetidyl, piperazinyl, piperidyl, homopiperazinyl, pyrrolidinyl, imidazolidinyl, pyrazolidinyl, tetrahydrofuryl, tetrahydrothienyl, tetrahydropyranyl, oxodihydropyridazinyl or oxetanyl radicals, all these radicals being optionally substituted; mention may in particular be made of the morpholinyl, thiomorpholinyl, homomorpholinyl, piperazinyl, piperidyl, homopiperazinyl or pyrrolidinyl radicals,
the terms aryl and heteroaryl denote unsaturated or partially unsaturated, respectively carbocyclic and heterocyclic, monocyclic or bicyclic radicals including at most 12 ring members which can optionally comprise a —C(O) ring member, the heterocyclic radicals comprising one or more identical or different heteroatoms chosen from O, N or S, with N, if appropriate, optionally substituted;
the term aryl radical thus denotes monocyclic or bicyclic radicals including from 6 to 12 ring members, such as, for example, the phenyl, naphthyl, biphenyl, indenyl, fluorenyl and anthracenyl radicals, more particularly the phenyl and naphthyl radicals and more particularly still the phenyl radical. It may be noted that a carbocyclic radical comprising a —C(O) ring member is, for example, the tetralonyl radical;
the term heteroaryl radical thus denotes monocyclic or bicyclic radicals including from 5 to 12 ring members: monocyclic heteroaryl radicals, such as, for example, the following radicals: thienyl, such as 2-thienyl and 3-thienyl, furyl, such as 2-furyl or 3-furyl, pyranyl, pyrrolyl, pyrrolinyl, pyrazolinyl, imidazolyl, pyrazolyl, pyridyl, such as 2-pyridyl, 3-pyridyl and 4-pyridyl, pyrazinyl, pyrimidinyl, pyridazinyl, oxazolyl, thiazolyl, isothiazolyl, diazolyl, thiadiazolyl, thiatriazolyl, oxadiazolyl, isoxazolyl, such as 3- or 4-isoxazolyl, furazanyl or tetrazolyl, free or salified, all these radicals being optionally substituted; including more particularly the following radicals: thienyl, such as 2-thienyl and 3-thienyl, furyl, such as 2-furyl, pyrrolyl, pyrrolinyl, pyrazolinyl, imidazolyl, pyrazolyl, oxazolyl, isoxazolyl, pyridyl or pyridazinyl, these radicals being optionally substituted; or bicyclic heteroaryl radicals, such as, for example, the following radicals: benzothienyl, such as 3-benzothienyl, benzothiazolyl, quinolyl, isoquinolyl, dihydroquinolyl, quinolonyl, tetralonyl, adamantyl, benzofuryl, isobenzofuryl, dihydrobenzofuryl, ethylenedioxyphenyl, thianthrenyl, benzopyrrolyl, benzimidazolyl, benzoxazolyl, thionaphthyl, indolyl, azaindolyl, indazolyl, purinyl, thienopyrazolyl, tetrahydroindazolyl, tetrahydrocyclopentapyrazolyl, dihydrofuropyrazolyl, tetrahydropyrrolopyrazolyl, oxotetrahydropyrrolopyrazolyl, tetrahydropyranopyrazolyl, tetrahydropyridinopyrazolyl, oxodihydropyridinopyrazolyl or dihydroimidazo[1,2-a]pyrazinyl, all these radicals being optionally substituted.

Mention may more particularly be made, as examples of heteroaryl or bicyclic radicals, of the pyrimidinyl, pyridyl, pyrrolyl, azaindolyl, indazolyl, pyrazolyl, benzothiazolyl or benzimidazolyl radicals, optionally substituted by one or more identical or different substituents, as indicated above.

The carboxyl radical or radicals of the products of formula (I) can be salified or esterified by various groups known to a person skilled in the art, among which may be mentioned, for example:
among the salification compounds, inorganic bases, such as, for example, one equivalent of sodium, potassium, lithium, calcium, magnesium or ammonium, or organic bases, such as, for example, methylamine, propylamine, trimethylamine, diethylamine, triethylamine, N,N-dimethylethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals in order to form alkoxycarbonyl groups, such as, for example, methoxycarbonyl, ethoxycarbonyl, tert-butoxycarbonyl or benzyloxycarbonyl, it being possible for these alkyl radicals to be substituted by radicals chosen, for example, from halogen atoms or hydroxyl, alkoxy, acyl, acyloxy, alkylthio, amino or aryl radicals, such as, for example, in the chloromethyl, hydroxypropyl, methoxymethyl, propionyloxymethyl, methylthiomethyl, dimethylaminoethyl, benzyl or phenethyl groups.

The addition salts with inorganic or organic acids of the products of formula (I) can, for example, be the salts formed with hydrochloric, hydrobromic, hydriodic, nitric, sulphuric, phosphoric, propionic, acetic, trifluoroacetic, formic, benzoic, maleic, fumaric, succinic, tartaric, citric, oxalic, glyoxylic, aspartic or ascorbic acid, alkylmonosulphonic acids, such as, for example, methanesulphonic acid, ethanesulphonic acid or propanesulphonic acid, alkyldisulphonic acids, such as, for example, methanedisulphonic acid or α,β-ethanedisulphonic acid, arylmonosulphonic acids, such as benzenesulphonic acid, and aryldisulphonic acids.

It may be remembered that stereoisomerism can be defined in its broad sense as the isomerism of compounds having the same expanded formulae but whose various groups are positioned differently in space, such as, in particular, in monosubstituted cyclohexanes, the substituent of which can be in the axial or equatorial position, and the different possible rotational conformations of ethane derivatives. However, there exists another type of stereoisomerism due to the different spatial arrangements of substituents attached either to double bonds or to rings, which is often referred to as geometrical isomerism or cis-trans isomerism. The term stereoisomers is used in the present patent application in its broadest sense and thus relates to all of the compounds indicated above.

When NR1R2 or NR3R4 forms a ring as defined above, such an amine-comprising ring can be chosen in particular from the azetidinyl, pyrrolidinyl, pyrazolidinyl, pyrazolinyl, piperidyl, azepinyl, morpholinyl, homomorpholinyl, piperazinyl or homopiperazinyl radicals, these radicals being themselves optionally substituted, as indicated above or below, for example by one or more identical or different radicals chosen from halogen atoms and alkyl, hydroxyl, alkoxy, phenyl and CH₂-phenyl radicals, the alkyl or phenyl radicals being themselves optionally substituted by one or more identical or different radicals chosen from halogen atoms and alkyl, hydroxyl, alkoxy, NH₂, NHalk and N(alk)₂ radicals.

The NR1R2 or NR3R4 ring can be chosen more particularly from the pyrrolidinyl radical, the morpholino radical, optionally substituted by one or two alkyl radicals, or the piperazinyl radical, optionally substituted on the second nitrogen atom by an alkyl, phenyl and CH₂-phenyl radical, themselves optionally substituted by one or more identical or different radicals chosen from halogen atoms and alkyl, hydroxyl and alkoxy radicals.

The present invention has more particularly as subject-matter the products of formula (I) as defined above in which:

n=0, 1 or 2;

X represents a hydrogen atom or a fluorine atom;

R represents a hydrogen atom or an NH₂ radical;

Ra represents a hydrogen atom, a —O-cycloalkyl radical, a —NH-cycloalkyl radical, a —NH-alk-phenyl radical or a phenyl radical, the phenyl radicals being optionally substituted by a halogen atom;

Rb represents a hydrogen atom, a —CO—Rc radical or a —CO—NRcRd radical;

with Rc representing a cycloalkyl radical optionally substituted by an alkyl radical itself optionally substituted by a morpholino radical; a heterocycloalkyl radical optionally substituted by an alkyl radical; a phenyl radical; or an alkyl radical substituted by an alkoxy, NR1R2 or heterocycloalkyl radical, itself optionally substituted by one or more radicals chosen from halogen atoms and alkyl radicals;

Rd represents a hydrogen atom;

NR1R2 being such that either R1 and R2, which are identical or different, represent a hydrogen atom or an alkyl radical or else NR1R2 represents the —NHCO₂alk radical; or R1 and R2 form, with the nitrogen atom to which they are bonded, a cyclic radical including from 4 to 7 ring members and optionally another heteroatom chosen from O, S, N and NH, optionally substituted by one or more identical or different radicals chosen from oxo, NH₂, NHalk and N(alk)₂ radicals and alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and alkyl, hydroxyl, alkoxy, NH₂, NHalk and N(alk)₂ radicals;

the alkyl or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention thus has as subject-matter the products of formula (I) as defined above in which:

Ra represents a hydrogen atom;

Rb represents a hydrogen atom, a —CO—Rc radical or a —CO—NRcRd radical;

with Rc representing a cyclopropyl radical or an alkyl radical optionally substituted by an alkoxy or NR1R2 radical;

Rd represents a hydrogen atom;

NR1R2 being such that either R1 and R2, which are identical or different, represent a hydrogen atom or an alkyl radical or R1 and R2 form, with the nitrogen atom to which they are bonded, a morpholinyl radical;

the alkyl or alkoxy radicals above including from 1 to 4 carbon atoms,

the said products of formula (I) being in all the isomeric forms possible, racemic, enantiomeric and diastereoisomeric, and also the addition salts with inorganic and organic acids or with inorganic and organic bases of the said products of formula (I).

The present invention has very particularly as subject-matter the products of formula (I) as defined above corresponding to the following formulae:
6-(imidazo[1,2-a]pyrimidin-3-ylsulphanyl)-1,3-benzothiazol-2-amine
N-[6-(imidazo[1,2-a]pyrimidin-3-ylsulphanyl)-1,3-benzothiazol-2-yl]cyclopropanecarboxamide
N-[6-(imidazo[1,2-a]pyrimidin-3-ylsulphanyl)-1,3-benzothiazol-2-yl]acetamide
1-[6-(imidazo[1,2-a]pyrimidin-3-ylsulphanyl)-1,3-benzothiazol-2-yl]-3-[2-(morpholin-4-yl)ethyl]urea
1-[6-(imidazo[1,2-a]pyrimidin-3-ylsulphanyl)-1,3-benzothiazol-2-yl]-3-[2-(pyrrolidin-1-yl)ethyl]urea
N-[6-(imidazo[1,2-a]pyrimidin-3-ylsulphinyl)-1,3-benzothiazol-2-yl]cyclopropanecarboxamide
N-[6-(imidazo[1,2-a]pyrimidin-3-ylsulphonyl)-1,3-benzothiazol-2-yl]cyclopropanecarboxamide
N-[6-(imidazo[1,2-a]pyrimidin-3-ylsulphanyl)-1,3-benzothiazol-2-yl]-3-(pyrrolidin-1-yl)propanamide
N-[6-(imidazo[1,2-a]pyrimidin-3-ylsulphanyl)-1,3-benzothiazol-2-yl]benzamide
N-[6-(imidazo[1,2-a]pyrimidin-3-ylsulphanyl)-1,3-benzothiazol-2-yl]-2-(4-methylpiperazin-1-yl)acetamide
2-methylpropan-2-yl(2-{[6-(imidazo[1,2-a]pyrimidin-3-ylsulphanyl)-1,3-benzothiazol-2-yl]amino}-2-oxoethyl)carbamate
N-[6-(imidazo[1,2-a]pyrimidin-3-ylsulphanyl)-1,3-benzothiazol-2-yl]glycinamide dihydrochloride
(trans-A)-N-[6-(imidazo[1,2-a]pyrimidin-3-ylsulphanyl)-1,3-benzothiazol-2-yl]-2-(morpholin-4-ylmethyl)cyclopropanecarboxamide
(trans-B)-N-[6-(imidazo[1,2-a]pyrimidin-3-ylsulphanyl)-1,3-benzothiazol-2-yl]-2-(morpholin-4-ylmethyl)cyclopropanecarboxamide
2-(4-ethylpiperazin-1-yl)-N-[6-(imidazo[1,2-a]pyrimidin-3-ylsulphanyl)-1,3-benzothiazol-2-yl]acetamide
2-(4-cyclopropylpiperazin-1-yl)-N-[6-(imidazo[1,2-a]pyrimidin-3-ylsulphanyl)-1,3-benzothiazol-2-yl]acetamide
N₂,N₂-diethyl-N-[6-(imidazo[1,2-a]pyrimidin-3-ylsulphanyl)-1,3-benzothiazol-2-yl]glycinamide
N-[5-fluoro-6-(imidazo[1,2-a]pyrimidin-3-ylsulphanyl)-1,3-benzothiazol-2-yl]cyclopropanecarboxamide
5-fluoro-6-(imidazo[1,2-a]pyrimidin-3-ylsulphanyl)-1,3-benzothiazol-2-amine
N-[6-(imidazo[1,2-a]pyrimidin-3-ylsulphanyl)-1,3-benzothiazol-2-yl]-3-methoxypropanamide
N-[6-(imidazo[1,2-a]pyrimidin-3-ylsulphanyl)-1,3-benzothiazol-2-yl]-2-(4-methyl-3-oxopiperazin-1-yl)acetamide
N-{6-[(7-aminoimidazo[1,2-a]pyrimidin-3-yl)sulphanyl]-1,3-benzothiazol-2-yl}cyclopropanecarboxamide
N-(6-{[6-(3-fluorophenyl)imidazo[1,2-a]pyrimidin-3-yl]sulphanyl}-1,3-benzothiazol-2-yl)cyclopropanecarboxamide
N-(6-{[6-(cyclohexyloxy)imidazo[1,2-a]pyrimidin-3-yl]sulphanyl}-1,3-benzothiazol-2-yl)cyclopropanecarboxamide
3-[(2-amino-1,3-benzothiazol-6-yl)sulphanyl]-N-cyclohexylimidazo[1,2-a]pyrimidin-6-amine
N-(6-{[6-(benzylamino)imidazo[1,2-a]pyrimidin-3-yl]sulphanyl}-1,3-benzothiazol-2-yl)cyclopropanecarboxamide
N-[6-(imidazo[1,2-a]pyrimidin-3-ylsulphanyl)-1,3-benzothiazol-2-yl]tetrahydro-2H-pyran-4-carboxamide

and the addition salts with inorganic and organic acids or with inorganic and organic bases of the said products of formula (I).

The present invention also relates to the following products of formula (I):
N-[6-(imidazo[1,2-a]pyrimidin-3-ylsulphanyl)-1,3-benzothiazol-2-yl]-3-(morpholin-4-yl)propanamide
N-[6-(imidazo[1,2-a]pyrimidin-3-ylsulphanyl)-1,3-benzothiazol-2-yl]-2-(morpholin-4-yl)acetamide
N-[6-(imidazo[1,2-a]pyrimidin-3-ylsulphanyl)-1,3-benzothiazol-2-yl]-3-(4-methylpiperazin-1-yl)propanamide
N-[6-(imidazo[1,2-a]pyrimidin-3-ylsulphanyl)-1,3-benzothiazol-2-yl]-2-[4-(propan-2-yl)piperazin-1-yl]acetamide
2-(4-cyclopropylpiperazin-1-yl)-N-[5-fluoro-6-(imidazo[1,2-a]pyrimidin-3-ylsulphanyl)-1,3-benzothiazol-2-yl]acetamide
N₂-ethyl-N-[6-(imidazo[1,2-a]pyrimidin-3-ylsulphanyl)-1,3-benzothiazol-2-yl]glycinamide
2-(4-cyclopropylpiperazin-1-yl)-N-[5-fluoro-6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-2-(4-methyl-1,4-diazepan-1-yl)acetamide
2-(4-ethyl-1,4-diazepan-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-3-[4-(2,2,2-trifluoroethyl)piperazin-1-yl]propanamide
N-[6-(imidazo[1,2-a]pyrimidin-3-ylsulphanyl)-1,3-benzothiazol-2-yl]-2-[4-(2,2,2-trifluoroethyl)piperazin-1-yl]acetamide
N-[6-(imidazo[1,2-a]pyrimidin-3-ylsulphanyl)-1,3-benzothiazol-2-yl]-2-(1-methylpiperidin-4-yl)acetamide
N-[6-(imidazo[1,2-a]pyrimidin-3-ylsulphanyl)-1,3-benzothiazol-2-yl]-3-(1-methylpiperidin-4-yl)propanamide
2-(3-fluoro-1-methylpiperidin-4-yl)-N-[6-(imidazo[1,2-a]pyrimidin-3-ylsulphanyl)-1,3-benzothiazol-2-yl]acetamide
3-(3-fluoro-1-methylpiperidin-4-yl)-N-[6-(imidazo[1,2-a]pyrimidin-3-ylsulphanyl)-1,3-benzothiazol-2-yl]propanamide
2-(3,3-difluoro-1-methylpiperidin-4-yl)-N-[6-(imidazo[1,2-a]pyrimidin-3-ylsulphanyl)-1,3-benzothiazol-2-yl]acetamide
3-(3,3-difluoro-1-methylpiperidin-4-yl)-N-[6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-1-methylazetidine-3-carboxamide
2-(3,5-dimethylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-(3,4,5-trimethylpiperazin-1-yl)acetamide
3-(3,5-dimethylpiperazin-1-yl)-N-[6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-3-(3,4,5-trimethylpiperazin-1-yl)propanamide
3-(5,6-dihydroimidazo[1,2-a]pyrazin-7(8H)-yl)-N-[6-(imidazo[1,2-a]pyrimidin-3-ylsulphanyl)-1,3-benzothiazol-2-yl]propanamide
2-(5,6-dihydroimidazo[1,2-a]pyrazin-7(8H)-yl)-N-[6-(imidazo[1,2-a]pyrimidin-3-ylsulphanyl)-1,3-benzothiazol-2-yl]acetamide
2-(4-cyclohexylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-[4-(tetrahydro-2H-pyran-4-yl)piperazin-1-yl]acetamide
N-[6-(imidazo[1,2-a]pyrimidin-3-ylsulphanyl)-1,3-benzothiazol-2-yl]-2-[4-(4-methyltetrahydro-2H-pyran-4-yl)piperazin-1-yl]acetamide
N-[6-(imidazo[1,2-a]pyrimidin-3-ylsulphanyl)-1,3-benzothiazol-2-yl]-2-[4-(2-methylpropan-2-yl)piperazin-1-yl]acetamide
2-[4-(diethylamino)piperidin-1-yl]-N-[6-(imidazo[1,2-a]pyrimidin-3-ylsulphanyl)-1,3-benzothiazol-2-yl]acetamide
2-[3-(diethylamino)pyrrolidin-1-yl]-N-[6-(imidazo[1,2-a]pyrimidin-3-ylsulphanyl)-1,3-benzothiazol-2-yl]acetamide
2-(4-acetylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-[4-(2-methoxyethyl)piperazin-1-yl]acetamide
2-[4-(2-hydroxyethyl)piperazin-1-yl]-N-[6-(imidazo[1,2-a]pyrimidin-3-ylsulphanyl)-1,3-benzothiazol-2-yl]acetamide
methyl 4-(2-{[6-(imidazo[1,2-a]pyrimidin-3-ylsulphanyl)-1,3-benzothiazol-2-yl]amino}-2-oxoethyl)piperazine-1-carboxylate
2-[4-(N,N-dimethylglycyl)piperazin-1-yl]-N-[6-(imidazo[1,2-a]pyrimidin-3-ylsulphanyl)-1,3-benzothiazol-2-yl]acetamide
N₂,N₂-diethyl-N-[6-(imidazo[1,2-a]pyrimidin-3-ylsulphanyl)-1,3-benzothiazol-2-yl]glycinamide
N-[6-(imidazo[1,2-a]pyrimidin-3-ylsulphanyl)-1,3-benzothiazol-2-yl]-2-(tetrahydropyran-4-yl)acetamide
N-[5-fluoro-6-(imidazo[1,2-a]pyrimidin-3-ylsulphanyl)-1,3-benzothiazol-2-yl]-2-(4-methyl-1,4-diazepan-1-yl)acetamide
2-(4-ethyl-1,4-diazepan-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-3-(4-methylpiperazin-1-yl)propanamide
N-[5-fluoro-6-(imidazo[1,2-a]pyrimidin-3-ylsulphanyl)-1,3-benzothiazol-2-yl]-3-[4-(2,2,2-trifluoroethyl)piperazin-1-yl]propanamide
N-[5-fluoro-6-(imidazo[1,2-a]pyrimidin-3-ylsulphanyl)-1,3-benzothiazol-2-yl]-2-[4-(2,2,2-trifluoroethyl)piperazin-1-yl]acetamide
N-[5-fluoro-6-(imidazo[1,2-a]pyrimidin-3-ylsulphanyl)-1,3-benzothiazol-2-yl]-2-(1-methylpiperidin-4-yl)acetamide
N-[5-fluoro-6-(imidazo[1,2-a]pyrimidin-3-ylsulphanyl)-1,3-benzothiazol-2-yl]-3-(1-methylpiperidin-4-yl)propanamide
N-[5-fluoro-6-(imidazo[1,2-a]pyrimidin-3-ylsulphanyl)-1,3-benzothiazol-2-yl]-2-(3-fluoro-1-methylpiperidin-4-yl)acetamide
N-[5-fluoro-6-(imidazo[1,2-a]pyrimidin-3-ylsulphanyl)-1,3-benzothiazol-2-yl]-3-(3-fluoro-1-methylpiperidin-4-yl)propanamide
2-(3,3-difluoro-1-methylpiperidin-4-yl)-N-[5-fluoro-6-(imidazo[1,2-a]pyrimidin-3-ylsulphanyl)-1,3-benzothiazol-2-yl]acetamide
3-(3,3-difluoro-1-methylpiperidin-4-yl)-N-[5-fluoro-6-(imidazo[1,2-a]pyrimidin-3-ylsulphanyl)-1,3-benzothiazol-2-yl]propanamide
N-[5-fluoro-6-(imidazo[1,2-a]pyrimidin-3-ylsulphanyl)-1,3-benzothiazol-2-yl]-1-methylazetidine-3-carboxamide
2-(3,5-dimethylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-(3,4,5-trimethylpiperazin-1-yl)acetamide
3-(3,5-dimethylpiperazin-1-yl)-N-[5-fluoro-6-(imidazo[1,2-a]pyrimidin-3-ylsulphanyl)-1,3-benzothiazol-2-yl]propanamide
N-[5-fluoro-6-(imidazo[1,2-a]pyrimidin-3-ylsulphanyl)-1,3-benzothiazol-2-yl]-3-(3,4,5-trimethylpiperazin-1-yl)propanamide
3-(5,6-dihydroimidazo[1,2-a]pyrazin-7(8H)-yl)-N-[5-fluoro-6-(imidazo[1,2-a]pyrimidin-3-ylsulphanyl)-1,3-benzothiazol-2-yl]propanamide
2-(5,6-dihydroimidazo[1,2-a]pyrazin-7(8H)-yl)-N-[5-fluoro-6-(imidazo[1,2-a]pyrimidin-3-ylsulphanyl)-1,3-benzothiazol-2-yl]acetamide
2-(4-cyclohexylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-[4-(tetrahydro-2H-pyran-4-yl)piperazin-1-yl]acetamide
N-[5-fluoro-6-(imidazo[1,2-a]pyrimidin-3-ylsulphanyl)-1,3-benzothiazol-2-yl]-2-[4-(4-methyltetrahydro-2H-pyran-4-yl)piperazin-1-yl]acetamide
N-[5-fluoro-6-(imidazo[1,2-a]pyrimidin-3-ylsulphanyl)-1,3-benzothiazol-2-yl]-2-[4-(2-methylpropan-2-yl)piperazin-1-yl]acetamide
2-[4-(diethylamino)piperidin-1-yl]-N-[5-fluoro-6-(imidazo[1,2-a]pyrimidin-3-ylsulphanyl)-1,3-benzothiazol-2-yl]acetamide
2-[3-(diethylamino)pyrrolidin-1-yl]-N-[5-fluoro-6-(imidazo[1,2-a]pyrimidin-3-ylsulphanyl)-1,3-benzothiazol-2-yl]acetamide
2-(4-acetylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-[4-(2-methoxyethyl)piperazin-1-yl]acetamide
N-[5-fluoro-6-(imidazo[1,2-a]pyrimidin-3-ylsulphanyl)-1,3-benzothiazol-2-yl]-2-[4-(2-hydroxyethyl)piperazin-1-yl]acetamide
methyl 4-(2-{[5-fluoro-6-(imidazo[1,2-a]pyrimidin-3-ylsulphanyl)-1,3-benzothiazol-2-yl]amino}-2-oxoethyl)piperazine-1-carboxylate
2-[4-(N,N-dimethylglycyl)piperazin-1-yl]-N-[5-fluoro-6-(imidazo[1,2-a]pyrimidin-3-ylsulphanyl)-1,3-benzothiazol-2-yl]acetamide
2-(4-cyclopropylpiperazin-1-yl)-N-[5-fluoro-6-(imidazo[1,2-a]pyrimidin-3-ylsulphanyl)-1,3-benzothiazol-2-yl]acetamide
N₂,N₂-diethyl-N-[5-fluoro-6-(imidazo[1,2-a]pyrimidin-3-ylsulphanyl)-1,3-benzothiazol-2-yl]glycinamide
N-[5-fluoro-6-(imidazo[1,2-a]pyrimidin-3-ylsulphanyl)-1,3-benzothiazol-2-yl]-2-[4-(propan-2-yl)piperazin-1-yl]acetamide

and to the addition salts with inorganic and organic acids or with inorganic and organic bases of the said products of formula (I).

A further subject-matter of the present invention is any process for the preparation of the products of formula (I) as defined above.

The products according to the invention can be prepared from conventional methods of organic chemistry.

Preparation of Compounds of Formula (I)

Schemes 1, 2 and 3 below are illustrative of the methods used for the preparation of the products of formula (I). As such, they should not constitute a limitation on the scope of the invention as regards the methods for the preparation of the compounds claimed.

The products of formula (I) as defined above according to the present invention can thus in particular be prepared according to the process described in Schemes 1, 2 and 3 below.

Another subject-matter of the present invention is thus the process for the preparation of products of formula (I) according to Scheme 1 as defined below.

Another subject-matter of the present invention is thus the process for the preparation of products of formula (I) according to Scheme 2 as defined below.

Another subject-matter of the present invention is thus the process for the preparation of products of formula (I) according to Scheme 3 as defined below.

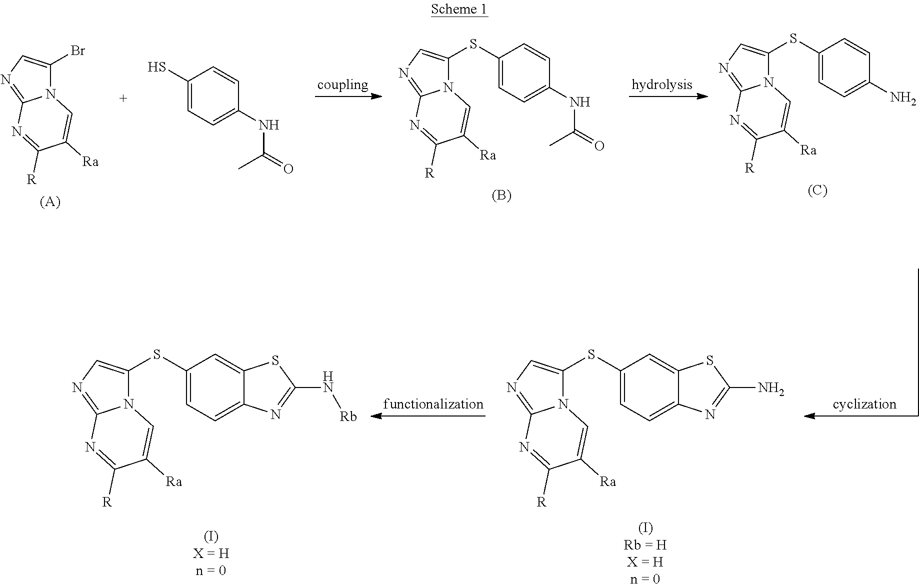

In Scheme 1 above, the Ra, Rb and R substituents have the meanings indicated above, X═H and n=0.

The compounds (I) for which Ra, Rb, R and X have the same meanings and n=0 can be obtained from the compounds (I) for which Rb═H.

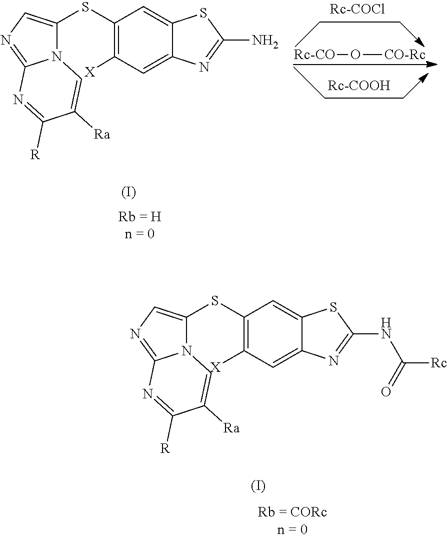

More particularly, the compounds (I) for which Rb═CORc (with Rc as defined above) can be obtained, for example:
by reaction of an acid chloride of formula Rc-COCl in the presence, for example, of a solvent, such as pyridine, at a temperature in the vicinity of 20° C.,
by reaction of an acid anhydride of formula Rc-CO—O—CO—Rc in the presence, for example, of a solvent, such as pyridine at a temperature in the vicinity of 20° C.,
by reaction with a carboxylic acid of formula Rc-COOH under the conditions, for example, described by D. DesMarteau et al. (Chem. Lett., 2000, 9, 1052), in the presence of 1-hydroxybenzotriazole and 1-(3-dimethylaminopropyl)-3-ethylcarbodiimide and in the presence of a base, such as triethylamine, at a temperature of between 20° C. and the reflux temperature of the solvent.

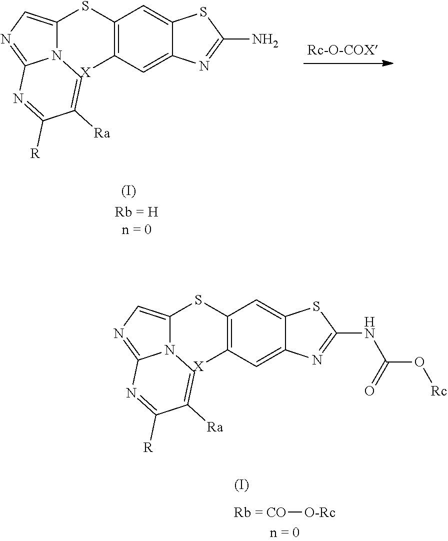

More particularly, the compounds (I) for which Rb═CO—O—Rc (with Rc as defined above) can be obtained, for example, by reaction with a chlorocarbonate Rc-O—COX′ (X′═Cl) of the compounds (I) for which Rb═H, in a solvent, such as tetrahydrofuran, in the presence of a base, such as sodium hydrogencarbonate, or in pyridine, at a temperature in the vicinity of 20° C.

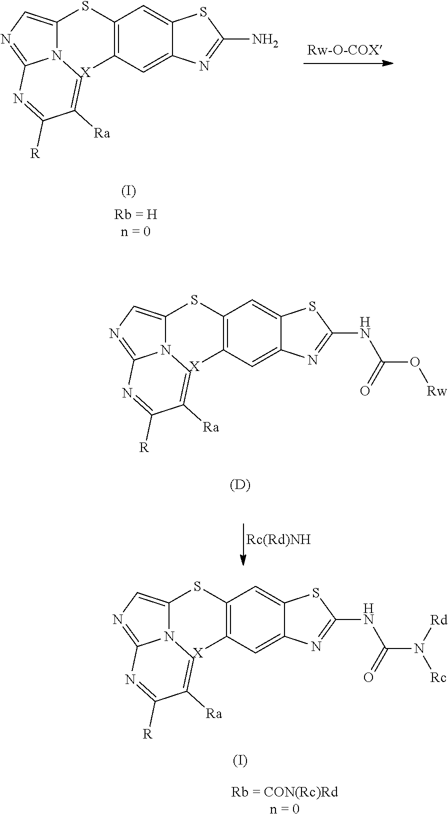

More particularly, the compounds (I) for which Rb═CON(Rc)Rd (with Rc and Rd as defined above) can be obtained, for example, by reaction of the carbamates (D), where Rw=phenyl, with amines Rc(Rd)NH (with Rc and Rd as defined above) in the presence of an aprotic solvent, such as tetrahydrofuran, at a temperature in the vicinity of 20° C.

The carbamates (D) can be obtained, for example, by reaction with a chlorocarbonate Rw-O—COX′0 (X′═Cl) of the compounds (I) for which Rb═H, in a solvent, such as tetrahydrofuran, in the presence of a base, such as sodium hydrogencarbonate, or in pyridine, at a temperature in the vicinity of 20° C.

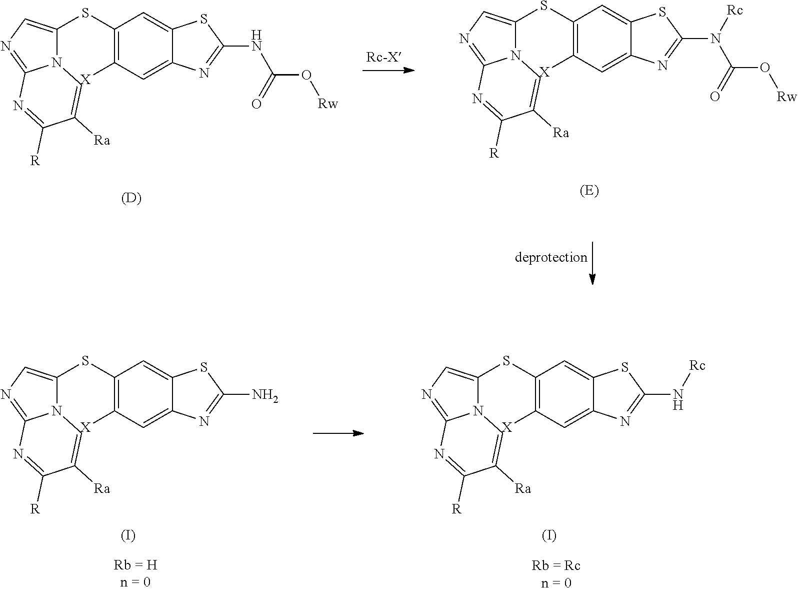

More particularly, the compounds (I) for which Rb═Rc (with Rc as defined above) can be obtained, for example:
by deprotection of the carbamates (E), with Rw=t-butyl, according to a method normal for a person skilled in the art, for example with trifluoroacetic acid in a solvent, such as dichloromethane, at a temperature in the vicinity of 20° C.,
from the compounds (I) for which Rb═H, by application of the methods described in Patent EP 0 408 437 or by R. A. Glennon et al. (Journal of Medicinal Chemistry, 1981, 24, 766-769).

The carbamates (E) can be obtained, for example, by reaction of the carbamates (D), where Rw=t-butyl, with halides Rc-X′ (with Rc as defined above) in the presence of a solvent, such as N,N-dimethylformamide, in the presence of a base, such as sodium hydride, at a temperature of between 20° C. and 90° C.

The compounds (I) for which Rb═H can be obtained by cyclization of the compounds (C) according to a method normal for a person skilled in the art, for example by application of the methods described by H. Masaichi et al. (Journal of Medicinal Chemistry, 2007, 50(18), 4453-4470), by reaction of potassium thiocyanate and bromine in the presence of acid, such as acetic acid, at a temperature of between 20° C. and the reflux temperature of the solvent.

The compounds (C) can be obtained by hydrolysis of the acetamide functional group of the compounds (B) according to a method normal for a person skilled in the art, for example using acid, such as hydrochloric acid, in a solvent, such as ethanol, at a temperature of between 20° C. and reflux of the solvent.

The compounds (B) can be obtained by coupling of the compounds (A), with Ra and R as defined above, with N-(4-sulphanylphenyl)acetamide (commercial product) under the conditions described, for example, by R. Varala et al. (Chemistry Letters, 2004, 33(12), 1614-1615), or by M. Winn et al. (Journal of Medicinal Chemistry, 2001, 44, 4393-4403), in the presence of a base, such as, for example, potassium carbonate, in a solvent, such as dimethyl sulphoxide, at a temperature of between 20° C. and the reflux temperature of the solvent. Such reactions can also be carried out under microwave irradiation.

The compounds (B) can also be obtained by coupling of the compounds (A) as described above with other 4-aminothiophenol derivatives, such as (4-NHR′)Ph-SH derivatives, where the amine functional group is free ((4-NH₂)Ph-SH, commercial product) or protected by a t-butyloxycarbonyl group, for example ((4-NHCO₂-t-Bu)Ph-SH, known product).

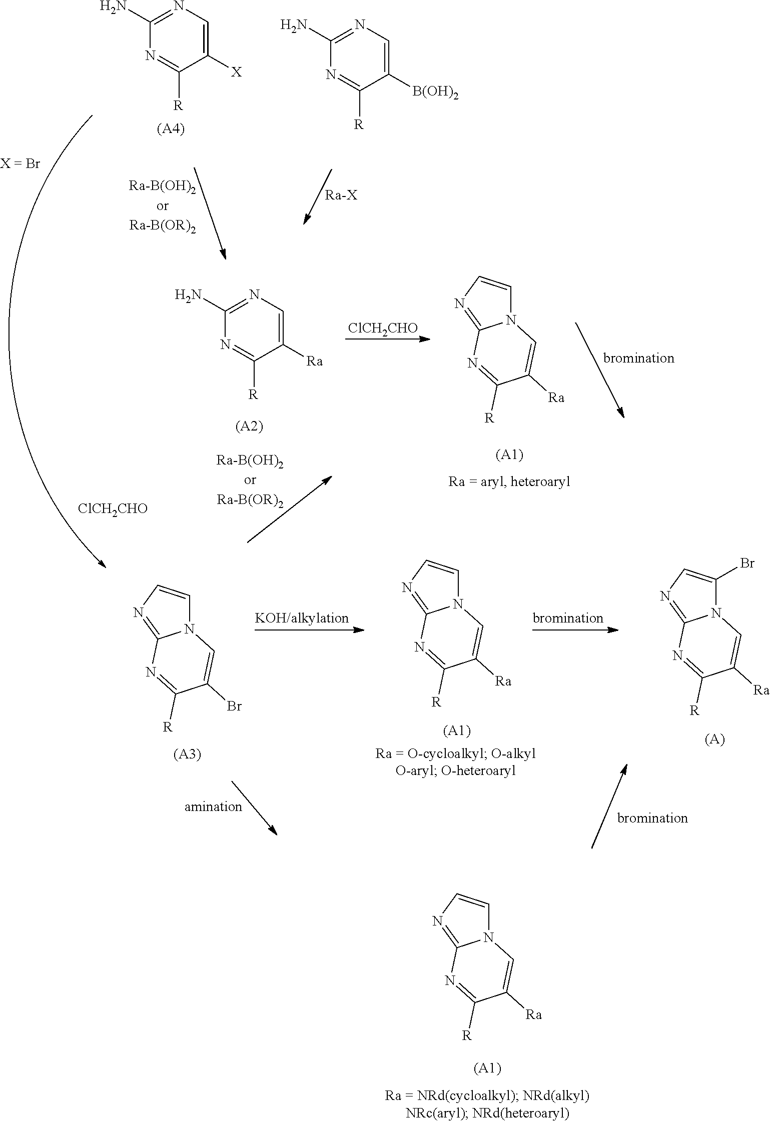

The compounds (A) are either commercially available or are prepared by bromination of the compounds (A1), according to a method normal for a person skilled in the art, for example according to the conditions described by S. C. Goodacre et al. (Journal of Medicinal Chemistry, 2006, 49(1), 35-38), using bromine or N-bromosuccinimide, in a solvent, such as ethanol or chloroform, at a temperature of between 20° C. and the temperature of the solvent.

The compounds (A1) are either commercially available or can be obtained according to a method normal for a person skilled in the art, for example by cyclization of the 2-aminopyrimidine compounds (A2) with chloroacetaldehyde, as described, for example, by Y. Rival et al. (European Journal of Medicinal Chemistry, 1991, 26, 13-18), in the presence of a base, such as sodium hydrogencarbonate, in a solvent, such as ethanol, at a temperature of between 20° C. and the reflux temperature of the solvent.

More particularly, the compounds (A1) for which Ra represents an aryl or heteroaryl radical can be obtained from the compounds (A3) by a coupling reaction with boronic acids Ra—B(OH)₂ or boronic esters Ra—B(OR)₂, in the presence of palladium-tetrakis(triphenylphosphine) and sodium carbonate, in a solvent, such as, for example, N,N-dimethylformamide, at a temperature in the vicinity of 150° C. under microwave irradiation.

More particularly, the compounds (A1) for which Ra represents an —O-cycloalkyl, —O-alkyl, —O-aryl and —O-heteroaryl radical can be obtained by treatment of the compounds (A3) with a base, such as, for example, potassium hydroxide, and a cycloalkyl, alkyl, aryl and heteroaryl halide respectively, in a solvent, such as, for example, ethanol, at a temperature in the vicinity of 135° C. under microwave irradiation.

More particularly, the compounds (A1) for which Ra represents an —NRd(cycloalkyl), —NRd(alkyl), —NRd(aryl) and —NRd(heteroaryl) radical can be obtained by amination of the compounds (A3) in a solvent, such as, for example, acetonitrile, at a temperature in the vicinity of 120° C. under microwave irradiation.

The compounds (A2) are either commercially available or can be obtained according to a method normal for a person skilled in the art.

More particularly, the compounds (A2) for which Ra represents an aryl or heteroaryl radical and R═H can be obtained, for example:
from 2-amino-5-halopyrimidines (commercially available products) by a coupling reaction with boronic acids Ra—B(OH)₂ or boronic esters Ra—B(OR)₂ by application of the methods described by Y. Gong et al. (Synlett, 2000, 6, 829-831) or M. Berlin et al. (Bioorganic & Medicinal Chemistry Letters, 2006, 16, 989-994),
from 5-(2-aminopyrimidine)boronic acid (commercially available products) by a coupling reaction with commercially available compounds Ra—X (X═I, Cl or Br) by application of the methods described by K. M. Clapham et al. (European Journal of Organic Chemistry, 2007, 34, 5712-5716).

The compounds (A3) are either commercially available (R═H) or can be obtained according to a method normal for a person skilled in the art, for example by cyclization of commercially available or known 2-aminopyrimidine compounds (A4) with chloroacetaldehyde, as described above.

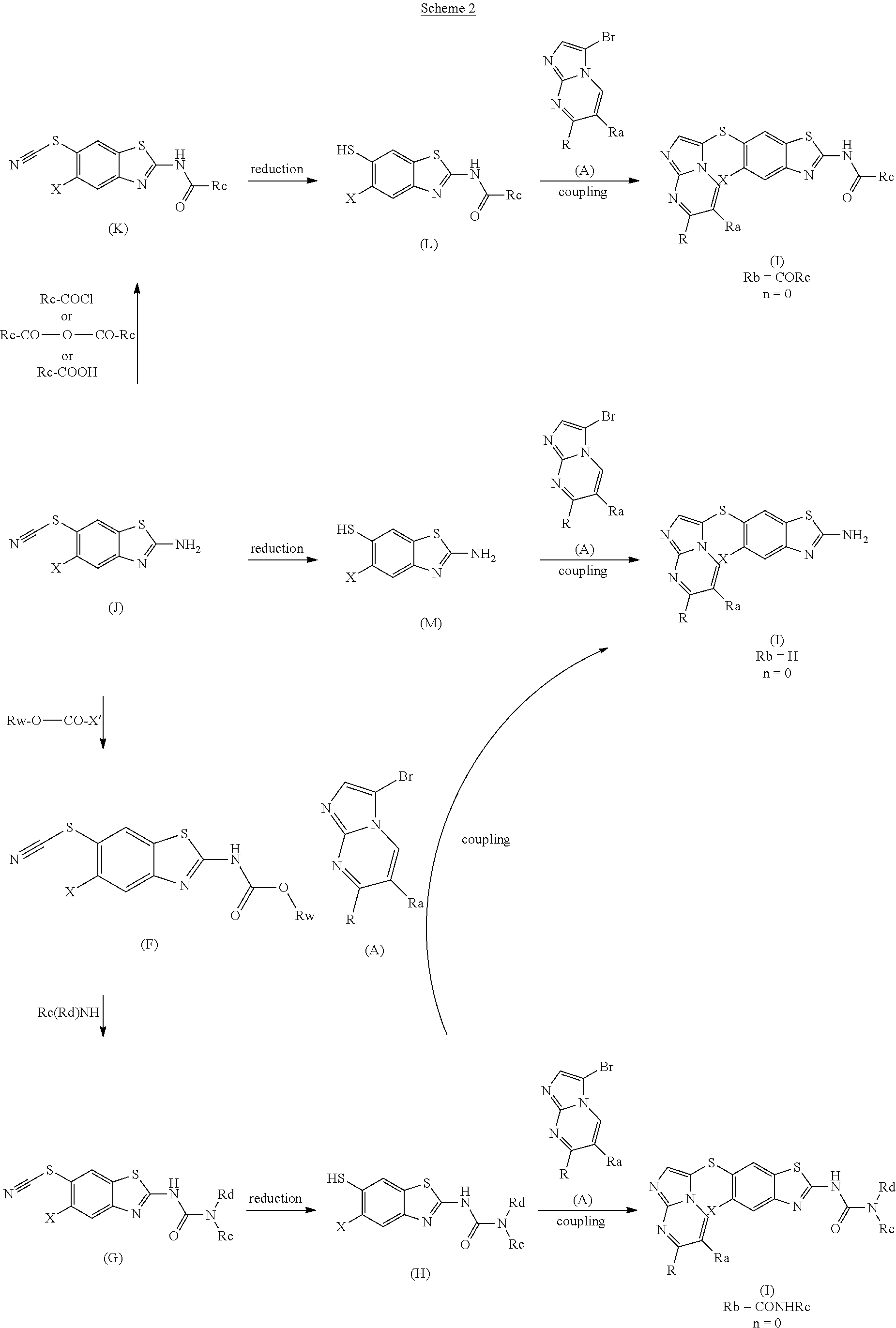

In Scheme 2 above, the Ra, Rc, Rd, R and X substituents have the meanings indicated above.

The compounds (I) for which Ra, R and X have the same meanings as above and for which Rb═H or Rb═CON(Rc)Rd can be obtained by a coupling reaction of the compounds (A), with Ra and R as defined above, with the compounds (H), with Rc, Rd and X as defined above, as described above for the preparation of the compounds (B).

The compounds (I) for which Ra, R and X have the same meanings as above and for which Rb═H can also be obtained by a coupling reaction of the compounds (A), with Ra and R as defined above, with the compounds (M), with X as defined above, as described above for the preparation of the compounds (B).

The compounds (I) for which Ra, R and X have the same meanings as above and for which Rb═CORc can be obtained by a coupling reaction of the compounds (A), with Ra and R as defined above, with the compounds (L), with Rc and X as defined above, as described above for the preparation of the compounds (B) or, for example, in the presence of bis(diphenylphosphino)-9,9-dimethylxanthene, tris(dibenzylideneacetone)dipalladium(0) and N,N-diisopropylethylamine, in a solvent, such as dimethylformamide, at a temperature of between 20° C. and the reflux temperature of the solvent. Such reactions can also be carried out under microwave irradiation.

The compounds (H), (L) and (M) for which Rc, Rd and X have the same meanings indicated above can be obtained, for example, from the compounds (G), (K) and (J) respectively by reduction with DL-dithiothreitol, in the presence of sodium hydrogencarbonate or potassium dihydrogenphosphate, in a solvent, such as ethanol, at a temperature of between 20° C. and reflux of the solvent.

The compounds (G) for which Rc, Rd and X have the same meanings indicated above can be obtained, for example, from the compounds (F) as described above for the preparation of the compounds (I) with Rb═CON(Rc)Rd.

The compounds (K) for which Rc and X have the same meanings indicated above can be obtained, for example, from the compounds (J) as described above for the preparation of the compounds (I) with Rb═CORc.

The compounds (F) can be obtained from the compounds (J) as described above for the preparation of the compounds (D).

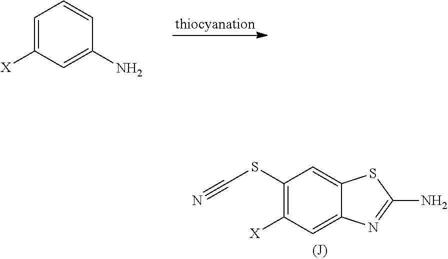

The compounds (J) for which X has the same meanings indicated above are either commercially available or can be prepared according to a method normal for a person skilled in the art, for example by thiocyanation of the corresponding anilines by reaction with potassium thiocyanate and bromine in the presence of acetic acid at a temperature of between 20° C. and the reflux temperature of the solvent or by reaction with sodium thiocyanate, sodium bromide and bromine in methanol, as described by J. V. N. Vara Prasad et al. (Tetrahedron Letters, 2000, 41, 4065-4068).

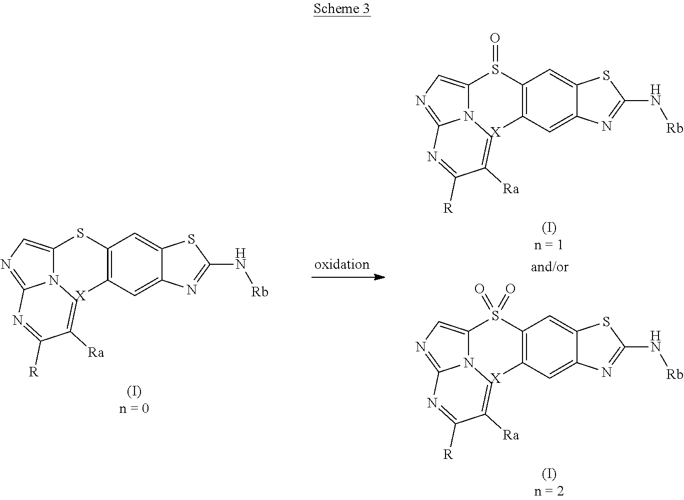

In Scheme 3 above, the Ra, Rb, R and X substituents have the meanings indicated above.

The compounds (I) for which Ra, Rb, R and X have the same meanings as above and for which n=1 or 2 can be obtained by oxidation of the compounds (I) for which n=0 according to a method normal for a person skilled in the art by using, for example, meta-chloroperbenzoic acid, in the presence of a solvent, such as dichloromethane, for example, at a temperature of between 20° C. and the reflux temperature of the solvent.

Among the starting materials of formulae (A), (A1), (A2), (A3), (A4), (F), (G) (J) and (K), some are known and can be obtained either commercially or according to the normal methods known to a person skilled in the art, for example starting from commercially available products.

It is understood, for a person skilled in the art, that, for the implementation of the processes according to the invention described above, it may be necessary to introduce protective groups for the amino, carboxyl and alcohol functional groups, in order to prevent side reactions.

The following nonexhaustive list of examples of the protection of reactive functional groups may be mentioned:
the hydroxyl groups can be protected, for example, with alkyl radicals, such as tert-butyl, trimethylsilyl, tert-butyldimethylsilyl, methoxymethyl, tetrahydropyranyl, benzyl or acetyl,
the amino groups can be protected, for example, with acetyl, trityl, benzyl, tert-butoxycarbonyl, BOC, benzyloxycarbonyl or phthalimido radicals or other radicals known in the chemistry of peptides,
the acid functional groups can be protected, for example in the form of esters formed with the easily cleavable esters, such as benzyl or tert-butyl esters, or esters known in the chemistry of peptides.

A list of various protective groups which can be used will be found in the handbooks known to a person skilled in the art and, for example in Patent BF 2 499 995.

It may be noted that it is possible to subject, if desired and if necessary, intermediates or products of formula (I) thus obtained by the processes indicated above, in order to obtain other intermediates or other products of formula (I), to one or more conversion reactions known to a person skilled in the art, such as, for example:

a) a reaction for the esterification of an acid functional group,

b) a reaction for the saponification of an ester functional group to give an acid functional group,

c) a reaction for the reduction of the free or esterified carboxyl functional group to give an alcohol functional group,

d) a reaction for the transformation of an alkoxy functional group to give a hydroxyl functional group or also of a hydroxyl functional group to give an alkoxy functional group,

e) a reaction for the removal of the protective groups which the protected reactive functional groups may carry,

f) a reaction for salification by an inorganic or organic acid or by a base in order to obtain the corresponding salt,

g) a reaction for the resolution of the racemic forms to give resolved products,

the said products of formula (I) thus obtained being in all the possible isomeric forms, racemic, enantiomeric and diastereoisomeric.

The reactions a) to g) can be carried out under the normal conditions known to a person skilled in the art, such as, for example, those indicated below.

a) The products described above can, if desired, form the subject, with regard to the possible carboxyl functional groups, of esterification reactions which can be carried out according to the normal methods known to a person skilled in the art.

b) The possible conversions of ester functional groups to give an acid functional group of the products described above can, if desired, be carried out under the normal conditions known to a person skilled in the art, in particular by acid or alkaline hydrolysis, for example by sodium hydroxide or potassium hydroxide in an alcoholic medium, such as, for example, in methanol, or also by hydrochloric or sulphuric acid.

The saponification reaction can be carried out according to the normal methods known to a person skilled in the art, such as, for example, in a solvent, such as methanol or ethanol, dioxane or dimethoxyethane, in the presence of sodium hydroxide or potassium hydroxide.

c) The possible free or esterified carboxyl functional groups of the products described above can, if desired, be reduced to give an alcohol functional group by the methods known to a person skilled in the art: the possible esterified carboxyl functional groups can, if desired, be reduced to give an alcohol functional group by the methods known to a person skilled in the art and in particular with lithium aluminium hydride in a solvent, such as, for example, tetrahydrofuran or dioxane or ethyl ether.

The possible free carboxyl functional groups of the products described above can, if desired, be reduced to give an alcohol functional group, in particular with boron hydride.

d) The possible alkoxy functional groups, such as, in particular, methoxy functional groups, of the products described above can, if desired, be converted to a hydroxyl functional group under the normal conditions known to a person skilled in the art, for example with boron tribromide in a solvent, such as, for example, methylene chloride, with pyridine hydrobromide or hydrochloride, or with hydrobromic or hydrochloric acid in water or trifluoroacetic acid, at reflux.

e) The removal of protective groups, such as, for example, those indicated above, can be carried out under the normal conditions known to a person skilled in the art, in particular by acid hydrolysis, carried out with an acid, such as hydrochloric, benzenesulphonic or para-toluenesulphonic, formic or trifluoroacetic acid, or by catalytic hydrogenation.

The phthalimido group can be removed with hydrazine.

f) The products described above can, if desired, form the subject of salification reactions, for example with an inorganic or organic acid or with an inorganic or organic base, according to the normal methods known to a person skilled in the art: such a salification reaction can be carried out, for example, in the presence of hydrochloric acid, for example, or of tartaric, citric or methanesulphonic acid, in an alcohol, such as, for example, ethanol or methanol.

g) The possible optically active forms of the products described above can be prepared by resolution of the racemates according to the normal methods known to a person skilled in the art.

The products of formula (I) as defined above and their addition salts with acids exhibit advantageous pharmacological properties, in particular due to their kinase-inhibiting properties, as is indicated above.

The products of the present invention are of use in particular in the therapeutic treatment of tumours.

The products of the invention can also thus enhance the therapeutic effects of antitumour agents currently used.

These properties justify their application in therapeutics and a subject-matter of the invention is in particular, as medicaments, the products of formula (I) as defined above, the said products of formula (I) being in all the possible isomeric forms, racemic, enantiomeric and diastereoisomeric, and the addition salts with pharmaceutically acceptable inorganic and organic acids or with pharmaceutically acceptable inorganic and organic bases of the said products of formula (I).

A subject-matter of the invention is very particularly, as medicaments, the products corresponding to the following formulae:
6-(imidazo[1,2-a]pyrimidin-3-ylsulphanyl)-1,3-benzothiazol-2-amine
N-[6-(imidazo[1,2-a]pyrimidin-3-ylsulphanyl)-1,3-benzothiazol-2-yl]cyclopropanecarboxamide
N-[6-(imidazo[1,2-a]pyrimidin-3-ylsulphanyl)-1,3-benzothiazol-2-yl]acetamide
1-[6-(imidazo[1,2-a]pyrimidin-3-ylsulphanyl)-1,3-benzothiazol-2-yl]-3-[2-(morpholin-4-yl)ethyl]urea
1-[6-(imidazo[1,2-a]pyrimidin-3-ylsulphanyl)-1,3-benzothiazol-2-yl]-3-[2-(pyrrolidin-1-yl)ethyl]urea
N-[6-(imidazo[1,2-a]pyrimidin-3-ylsulphinyl)-1,3-benzothiazol-2-yl]cyclopropanecarboxamide
N-[6-(imidazo[1,2-a]pyrimidin-3-ylsulphonyl)-1,3-benzothiazol-2-yl]cyclopropanecarboxamide
N-[6-(imidazo[1,2-a]pyrimidin-3-ylsulphanyl)-1,3-benzothiazol-2-yl]-3-(pyrrolidin-1-yl)propanamide
N-[6-(imidazo[1,2-a]pyrimidin-3-ylsulphanyl)-1,3-benzothiazol-2-yl]benzamide
N-[6-(imidazo[1,2-a]pyrimidin-3-ylsulphanyl)-1,3-benzothiazol-2-yl]-2-(4-methylpiperazin-1-yl)acetamide
2-methylpropan-2-yl(2-{[6-(imidazo[1,2-a]pyrimidin-3-ylsulphanyl)-1,3-benzothiazol-2-yl]amino}-2-oxoethyl)carbamate
N-[6-(imidazo[1,2-a]pyrimidin-3-ylsulphanyl)-1,3-benzothiazol-2-yl]glycinamide dihydrochloride
(trans-A)-N-[6-(imidazo[1,2-a]pyrimidin-3-ylsulphanyl)-1,3-benzothiazol-2-yl]-2-(morpholin-4-ylmethyl)cyclopropanecarboxamide
(trans-B)-N-[6-(imidazo[1,2-a]pyrimidin-3-ylsulphanyl)-1,3-benzothiazol-2-yl]-2-(morpholin-4-ylmethyl)cyclopropanecarboxamide
2-(4-ethylpiperazin-1-yl)-N-[6-(imidazo[1,2-a]pyrimidin-3-ylsulphanyl)-1,3-benzothiazol-2-yl]acetamide
2-(4-cyclopropylpiperazin-1-yl)-N-[6-(imidazo[1,2-a]pyrimidin-3-ylsulphanyl)-1,3-benzothiazol-2-yl]acetamide
N₂,N₂-diethyl-N-[6-(imidazo[1,2-a]pyrimidin-3-ylsulphanyl)-1,3-benzothiazol-2-yl]glycinamide
N-[5-fluoro-6-(imidazo[1,2-a]pyrimidin-3-ylsulphanyl)-1,3-benzothiazol-2-yl]cyclopropanecarboxamide
5-fluoro-6-(imidazo[1,2-a]pyrimidin-3-ylsulphanyl)-1,3-benzothiazol-2-amine
N-[6-(imidazo[1,2-a]pyrimidin-3-ylsulphanyl)-1,3-benzothiazol-2-yl]-3-methoxypropanamide
N-[6-(imidazo[1,2-a]pyrimidin-3-ylsulphanyl)-1,3-benzothiazol-2-yl]-2-(4-methyl-3-oxopiperazin-1-yl)acetamide
N-{6-[(7-aminoimidazo[1,2-a]pyrimidin-3-yl)sulphanyl]-1,3-benzothiazol-2-yl}cyclopropanecarboxamide
N-(6-{[6-(3-fluorophenyl)imidazo[1,2-a]pyrimidin-3-yl]sulphanyl}-1,3-benzothiazol-2-yl)cyclopropanecarboxamide
N-(6-{[6-(cyclohexyloxy)imidazo[1,2-a]pyrimidin-3-yl]sulphanyl}-1,3-benzothiazol-2-yl)cyclopropanecarboxamide
3-[(2-amino-1,3-benzothiazol-6-yl)sulphanyl]-N-cyclohexylimidazo[1,2-a]pyrimidin-6-amine
N-(6-{[6-(benzylamino)imidazo[1,2-a]pyrimidin-3-yl]sulphanyl}-1,3-benzothiazol-2-yl)cyclopropanecarboxamide
N-[6-(imidazo[1,2-a]pyrimidin-3-ylsulphanyl)-1,3-benzothiazol-2-yl]tetrahydro-2H-pyran-4-carboxamide

and the addition salts with pharmaceutically acceptable inorganic and organic acids or with pharmaceutically acceptable inorganic and organic bases of the said products of formula (I).

The present invention also relates, as medicaments, to the following products of formula (I):
N-[6-(imidazo[1,2-a]pyrimidin-3-ylsulphanyl)-1,3-benzothiazol-2-yl]-3-(morpholin-4-yl)propanamide
N-[6-(imidazo[1,2-a]pyrimidin-3-ylsulphanyl)-1,3-benzothiazol-2-yl]-2-(morpholin-4-yl)acetamide
N-[6-(imidazo[1,2-a]pyrimidin-3-ylsulphanyl)-1,3-benzothiazol-2-yl]-3-(4-methylpiperazin-1-yl)propanamide
N-[6-(imidazo[1,2-a]pyrimidin-3-ylsulphanyl)-1,3-benzothiazol-2-yl]-2-[4-(propan-2-yl)piperazin-1-yl]acetamide
2-(4-cyclopropylpiperazin-1-yl)-N-[5-fluoro-6-(imidazo[1,2-a]pyrimidin-3-ylsulphanyl)-1,3-benzothiazol-2-yl]acetamide
N₂-ethyl-N-[6-(imidazo[1,2-a]pyrimidin-3-ylsulphanyl)-1,3-benzothiazol-2-yl]glycinamide
2-(4-cyclopropylpiperazin-1-yl)-N-[5-fluoro-6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-2-(4-methyl-1,4-diazepan-1-yl)acetamide
2-(4-ethyl-1,4-diazepan-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-3-[4-(2,2,2-trifluoroethyl)piperazin-1-yl]propanamide
N-[6-(imidazo[1,2-a]pyrimidin-3-ylsulphanyl)-1,3-benzothiazol-2-yl]-2-[4-(2,2,2-trifluoroethyl)piperazin-1-yl]acetamide
N-[6-(imidazo[1,2-a]pyrimidin-3-ylsulphanyl)-1,3-benzothiazol-2-yl]-2-(1-methylpiperidin-4-yl)acetamide
N-[6-(imidazo[1,2-a]pyrimidin-3-ylsulphanyl)-1,3-benzothiazol-2-yl]-3-(1-methylpiperidin-4-yl)propanamide
2-(3-fluoro-1-methylpiperidin-4-yl)-N-[6-(imidazo[1,2-a]pyrimidin-3-ylsulphanyl)-1,3-benzothiazol-2-yl]acetamide
3-(3-fluoro-1-methylpiperidin-4-yl)-N-[6-(imidazo[1,2-a]pyrimidin-3-ylsulphanyl)-1,3-benzothiazol-2-yl]propanamide
2-(3,3-difluoro-1-methylpiperidin-4-yl)-N-[6-(imidazo[1,2-a]pyrimidin-3-ylsulphanyl)-1,3-benzothiazol-2-yl]acetamide
3-(3,3-difluoro-1-methylpiperidin-4-yl)-N-[6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-1-methylazetidine-3-carboxamide
2-(3,5-dimethylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-(3,4,5-trimethylpiperazin-1-yl)acetamide
3-(3,5-dimethylpiperazin-1-yl)-N-[6-(imidazo[1,2-a]pyrimidin-3-ylsulphanyl)-1,3-benzothiazol-2-yl]propanamide
N-[6-(imidazo[1,2-a]pyrimidin-3-ylsulphanyl)-1,3-benzothiazol-2-yl]-3-(3,4,5-trimethylpiperazin-1-yl)propanamide
3-(5,6-dihydroimidazo[1,2-a]pyrazin-7(8H)-yl)-N-[6-(imidazo[1,2-a]pyrimidin-3-ylsulphanyl)-1,3-benzothiazol-2-yl]propanamide
2-(5,6-dihydroimidazo[1,2-a]pyrazin-7(8H)-yl)-N-[6-(imidazo[1,2-a]pyrimidin-3-ylsulphanyl)-1,3-benzothiazol-2-yl]acetamide
2-(4-cyclohexylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-[4-(tetrahydro-2H-pyran-4-yl)piperazin-1-yl]acetamide
N-[6-(imidazo[1,2-a]pyrimidin-3-ylsulphanyl)-1,3-benzothiazol-2-yl]-2-[4-(4-methyltetrahydro-2H-pyran-4-yl)piperazin-1-yl]acetamide
N-[6-(imidazo[1,2-a]pyrimidin-3-ylsulphanyl)-1,3-benzothiazol-2-yl]-2-[4-(2-methylpropan-2-yl)piperazin-1-yl]acetamide
2-[4-(diethylamino)piperidin-1-yl]-N-[6-(imidazo[1,2-a]pyrimidin-3-ylsulphanyl)-1,3-benzothiazol-2-yl]acetamide
2-[3-(diethylamino)pyrrolidin-1-yl]-N-[6-(imidazo[1,2-a]pyrimidin-3-ylsulphanyl)-1,3-benzothiazol-2-yl]acetamide
2-(4-acetylpiperazin-1-yl)-N-[6-(imidazo[1,2-a]pyrimidin-3-ylsulphanyl)-1,3-benzothiazol-2-yl]acetamide
N-[6-(imidazo[1,2-a]pyrimidin-3-ylsulphanyl)-1,3-benzothiazol-2-yl]-2-[4-(2-methoxyethyl)piperazin-1-yl]acetamide
2-[4-(2-hydroxyethyl)piperazin-1-yl]-N-[6-(imidazo[1,2-a]pyrimidin-3-ylsulphanyl)-1,3-benzothiazol-2-yl]acetamide
methyl 4-(2-{[6-(imidazo[1,2-a]pyrimidin-3-ylsulphanyl)-1,3-benzothiazol-2-yl]amino}-2-oxoethyl)piperazine-1-carboxylate
2-[4-(N,N-dimethylglycyl)piperazin-1-yl]-N-[6-(imidazo[1,2-a]pyrimidin-3-ylsulphanyl)-1,3-benzothiazol-2-yl]acetamide
N₂,N₂-diethyl-N-[6-(imidazo[1,2-a]pyrimidin-3-ylsulphanyl)-1,3-benzothiazol-2-yl]glycinamide
N-[6-(imidazo[1,2-a]pyrimidin-3-ylsulphanyl)-1,3-benzothiazol-2-yl]-2-(tetrahydropyran-4-yl)acetamide
N-[5-fluoro-6-(imidazo[1,2-a]pyrimidin-3-ylsulphanyl)-1,3-benzothiazol-2-yl]-2-(4-methyl-1,4-diazepan-1-yl)acetamide
2-(4-ethyl-1,4-diazepan-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-3-(4-methylpiperazin-1-yl)propanamide
N-[5-fluoro-6-(imidazo[1,2-a]pyrimidin-3-ylsulphanyl)-1,3-benzothiazol-2-yl]-3-[4-(2,2,2-trifluoroethyl)piperazin-1-yl]propanamide
N-[5-fluoro-6-(imidazo[1,2-a]pyrimidin-3-ylsulphanyl)-1,3-benzothiazol-2-yl]-2-[4-(2,2,2-trifluoroethyl)piperazin-1-yl]acetamide
N-[5-fluoro-6-(imidazo[1,2-a]pyrimidin-3-ylsulphanyl)-1,3-benzothiazol-2-yl]-2-(1-methylpiperidin-4-yl)acetamide
N-[5-fluoro-6-(imidazo[1,2-a]pyrimidin-3-ylsulphanyl)-1,3-benzothiazol-2-yl]-3-(1-methylpiperidin-4-yl)propanamide
N-[5-fluoro-6-(imidazo[1,2-a]pyrimidin-3-ylsulphanyl)-1,3-benzothiazol-2-yl]-2-(3-fluoro-1-methylpiperidin-4-yl)acetamide
N-[5-fluoro-6-(imidazo[1,2-a]pyrimidin-3-ylsulphanyl)-1,3-benzothiazol-2-yl]-3-(3-fluoro-1-methylpiperidin-4-yl)propanamide
2-(3,3-difluoro-1-methylpiperidin-4-yl)-N-[5-fluoro-6-(imidazo[1,2-a]pyrimidin-3-ylsulphanyl)-1,3-benzothiazol-2-yl]acetamide
3-(3,3-difluoro-1-methylpiperidin-4-yl)-N-[5-fluoro-6-(imidazo[1,2-a]pyrimidin-3-ylsulphanyl)-1,3-benzothiazol-2-yl]propanamide
N-[5-fluoro-6-(imidazo[1,2-a]pyrimidin-3-ylsulphanyl)-1,3-benzothiazol-2-yl]-1-methylazetidine-3-carboxamide
2-(3,5-dimethylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-(3,4,5-trimethylpiperazin-1-yl)acetamide
3-(3,5-dimethylpiperazin-1-yl)-N-[5-fluoro-6-(imidazo[1,2-a]pyrimidin-3-ylsulphanyl)-1,3-benzothiazol-2-yl]propanamide
N-[5-fluoro-6-(imidazo[1,2-a]pyrimidin-3-ylsulphanyl)-1,3-benzothiazol-2-yl]-3-(3,4,5-trimethylpiperazin-1-yl)propanamide
3-(5,6-dihydroimidazo[1,2-a]pyrazin-7(8H)-yl)-N-[5-fluoro-6-(imidazo[1,2-a]pyrimidin-3-ylsulphanyl)-1,3-benzothiazol-2-yl]propanamide
2-(5,6-dihydroimidazo[1,2-a]pyrazin-7(8H)-yl)-N-[5-fluoro-6-(imidazo[1,2-a]pyrimidin-3-ylsulphanyl)-1,3-benzothiazol-2-yl]acetamide
2-(4-cyclohexylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-[4-(tetrahydro-2H-pyran-4-yl)piperazin-1-yl]acetamide
N-[5-fluoro-6-(imidazo[1,2-a]pyrimidin-3-ylsulphanyl)-1,3-benzothiazol-2-yl]-2-[4-(4-methyltetrahydro-2H-pyran-4-yl)piperazin-1-yl]acetamide
N-[5-fluoro-6-(imidazo[1,2-a]pyrimidin-3-ylsulphanyl)-1,3-benzothiazol-2-yl]-2-[4-(2-methylpropan-2-yl)piperazin-1-yl]acetamide
2-[4-(diethylamino)piperidin-1-yl]-N-[5-fluoro-6-(imidazo[1,2-a]pyrimidin-3-ylsulphanyl)-1,3-benzothiazol-2-yl]acetamide
2-[3-(diethylamino)pyrrolidin-1-yl]-N-[5-fluoro-6-(imidazo[1,2-a]pyrimidin-3-ylsulphanyl)-1,3-benzothiazol-2-yl]acetamide
2-(4-acetylpiperazin-1-yl)-N-[5-fluoro-6-(imidazo[1,2-a]pyrimidin-3-ylsulphanyl)-1,3-benzothiazol-2-yl]acetamide
N-[5-fluoro-6-(imidazo[1,2-a]pyrimidin-3-ylsulphanyl)-1,3-benzothiazol-2-yl]-2-[4-(2-methoxyethyl)piperazin-1-yl]acetamide
N-[5-fluoro-6-(imidazo[1,2-a]pyrimidin-3-ylsulphanyl)-1,3-benzothiazol-2-yl]-2-[4-(2-hydroxyethyl)piperazin-1-yl]acetamide
methyl 4-(2-{[5-fluoro-6-(imidazo[1,2-a]pyrimidin-3-ylsulphanyl)-1,3-benzothiazol-2-yl]amino}-2-oxoethyl)piperazine-1-carboxylate
2-[4-(N,N-dimethylglycyl)piperazin-1-yl]-N-[5-fluoro-6-(imidazo[1,2-a]pyrimidin-3-ylsulphanyl)-1,3-benzothiazol-2-yl]acetamide
2-(4-cyclopropylpiperazin-1-yl)-N-[5-fluoro-6-(imidazo[1,2-a]pyrimidin-3-ylsulphanyl)-1,3-benzothiazol-2-yl]acetamide
N₂,N₂-diethyl-N-[5-fluoro-6-(imidazo[1,2-a]pyrimidin-3-ylsulphanyl)-1,3-benzothiazol-2-yl]glycinamide
N-[5-fluoro-6-(imidazo[1,2-a]pyrimidin-3-ylsulphanyl)-1,3-benzothiazol-2-yl]-2-[4-(propan-2-yl)piperazin-1-yl]acetamide

and to the addition salts with pharmaceutically acceptable inorganic and organic acids or with pharmaceutically acceptable inorganic and organic bases of the said products of formula (I).

The invention also relates to pharmaceutical compositions comprising, as active principle, one at least of the products of formula (I) as defined above or a pharmaceutically acceptable salt of this product or a prodrug of this product and, if appropriate, a pharmaceutically acceptable carrier.

The invention thus applies to the pharmaceutical compositions comprising, as active principle, one at least of the medicaments as defined above.

Such pharmaceutical compositions of the present invention can also, if appropriate, include active principles of other antimitotic medicaments, such as, in particular, those based on taxol, cisplatin, DNA intercalating agents and others.

These pharmaceutical compositions can be administered orally, parenterally or locally by topical application to the skin and the mucous membranes or by intravenous or intramuscular injection.

These compositions can be solid or liquid and be provided in all the pharmaceutical forms commonly used in human medicine, such as, for example, simple or sugar-coated tablets, pills, lozenges, hard gelatin capsules, drops, granules, injectable preparations, ointments, creams or gels; they are prepared according to the usual methods. The active principle can be incorporated therein in excipients normally employed in these pharmaceutical compositions, such as talc, gum arabic, lactose, starch, magnesium stearate, cocoa butter, aqueous or nonaqueous carriers, fatty substances of animal or vegetable origin, paraffinic derivatives, glycols, various wetting, dispersing or emulsifying agents, or preservatives.

The usual dosage, which can vary according to the product used, the subject treated and the condition in question, can, for example, be from 0.05 to 5 g per day for adults or preferably from 0.1 to 2 g per day.

Another subject-matter of the present invention is the use of the products of formula (I) as defined above or of pharmaceutically acceptable salts of these products in the preparation of a medicament intended to inhibit the activity of a protein kinase.

Another subject-matter of the present invention is the use of products of formula (I) as defined above in the preparation of a medicament intended for the treatment or prevention of a disease characterized by deregulation of the activity of a protein kinase.

Such a medicament can in particular be intended for the treatment or prevention of a disease in a mammal.

Another subject-matter of the present invention is the use defined above in which the protein kinase is a protein tyrosine kinase.

Another subject-matter of the present invention is the use defined above in which the protein tyrosine kinase is MET or its mutant forms.

Another subject-matter of the present invention is the use defined above in which the protein kinase is in a cell culture.

Another subject-matter of the present invention is the use defined above in which the protein kinase is in a mammal.

A subject-matter of the present invention is in particular the use of a product of formula (I) as defined above in the preparation of a medicament intended for the prevention or treatment of diseases related to an uncontrolled proliferation.

A subject-matter of the present invention is in particular the use of a product of formula (I) as defined above in the preparation of a medicament intended for the treatment or prevention of a disease chosen from the following group: blood vessel proliferation disorders, fibrotic disorders, “mesangial” cell proliferation disorders, metabolic disorders, allergies, asthma, thrombosis, diseases of the nervous system, retinopathy, psoriasis, rheumatoid arthritis, diabetes, muscle degeneration and cancers.

A subject-matter of the present invention is thus very particularly the use of a product of formula (I) as defined above in the preparation of a medicament intended for the treatment or prevention of diseases in oncology and in particular intended for the treatment of cancers.

Among these cancers, interest is directed at the treatment of solid or liquid tumours and at the treatment of cancers which are resistant to cytotoxic agents.

The cited products of the present invention can in particular be used in the treatment of primary tumours and/or of metastases, in particular in gastric, liver, kidney, ovarian, colon, prostate or lung (NSCLC and SCLC) cancers, glioblastomas, thyroid, bladder or breast cancers, in melanomas, in lymphoid or myeloid haematopoietic tumours, in sarcomas and in brain, laryngeal, lymphatic, bone and pancreatic cancers.

Another subject-matter of the present invention is the use of the products of formula (I) as defined above in the preparation of medicaments intended for cancer chemotherapy.

Such medicaments intended for cancer chemotherapy can be used alone or in combination.

The products of the present patent application can in particular be administered alone or in combination with chemotherapy or radiotherapy or also in combination, for example, with other therapeutic agents.

Such therapeutic agents can be commonly used antitumour agents.

Mention may be made, as kinase inhibitors, of butyrolactone, flavopiridol and 2-(2-hydroxyethylamino)-6-benzylamino-9-methylpurine, known as olomoucine.

A further subject-matter of the present invention is, as novel industrial products, the synthetic intermediates of formulae (A), (B), (C), (D), (E), (F), (G), (H), (J), (K), (L) and (M) as defined above and restated below:

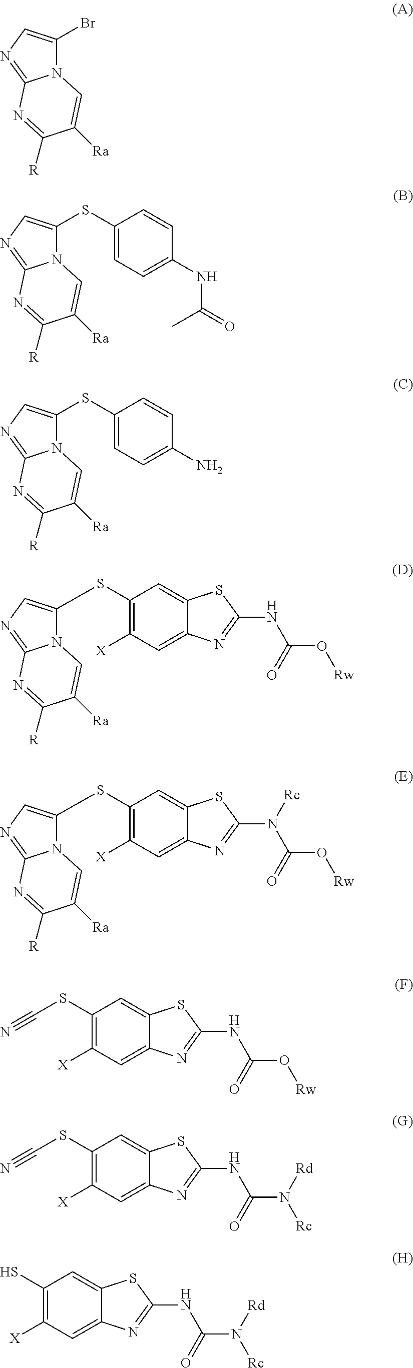

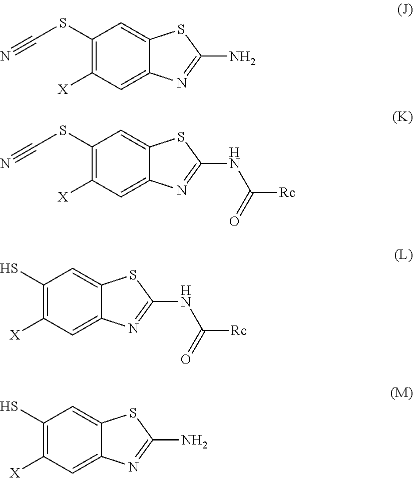

in which Ra, Rb, Rc, Rd, R and X have the definitions indicated above and Rw represents a t-butyl or phenyl radical.

The following examples, which are products of formula (I), illustrate the invention, without, however, limiting it.

Experimental Part

The nomenclature of the compounds of this present invention was carried out with the ACDLABS software, version 10.0.

The microwave oven used is a Biotage Initiator™ 2.0 device, 400 W max, 2450 MHz.

The 400 MHz ¹H NMR spectra were recorded on a Bruker Avance DRX-400 spectrometer with the chemical shifts (δ in ppm) in the solvent d₆-dimethyl sulphoxide (d₆-DMSO) referenced at 2.5 ppm at a temperature of 303K.

The mass spectra (MS) were obtained either by method A or by method B:

Method A:

Device Waters UPLC-SQD; Ionization: positive and/or negative mode electrospray (ES+/−); Chromatography conditions: Column: Acquity BEH C₁₈ 1.7 μm-2.1×50 mm; Solvents: A: H₂O (0.1% formic acid) B: CH₃CN (0.1% formic acid); Column temperature: 50° C.; Flow rate: 1 ml/min; Gradient (2 min): from 5 to 50% of B in 0.8 min; 1.2 min: 100% of B; 1.85 min: 100% of B; 1.95: 5% of B; Retention time=Tr (min).

Method B:

Device Waters ZQ; Ionization: positive and/or negative mode electrospray (ES+/−); Chromatography conditions: Column: XBridge C₁₈ 2.5 μm-3×50 mm; Solvents: A: H₂O (0.1% formic acid) B: CH₃CN (0.1% formic acid); Column temperature: 70° C.; Flow rate: 0.9 ml/min; Gradient (7 min): from 5 to 100% of B in 5.3 min; 5.5 min: 100% of B; 6.3 min: 5% of B; Retention time=Tr (min).

### EXAMPLE 1

### 6-(imidazo[1,2-a]pyrimidin-3-ylsulphanyl)-1,3-benzothiazol-2-amine

### EXAMPLE 1a

### 6-(imidazo[1,2-a]pyrimidin-3-ylsulphanyl)-1,3-benzothiazol-2-amine

The compound can be prepared in the following way:

600 mg of 3-bromoimidazo[1,2-a]pyrimidine (commercially available product), 1.05 g of 1-[2-(morpholin-4-yl)ethyl]-3-(6-sulphanyl-1,3-benzothiazol-2-yl)urea, 840 mg of potassium carbonate and 12 ml of dimethyl sulphoxide are charged to a sealed glass tube. The medium is heated at 190° C. for 12 minutes using microwave radiation. After returning to a temperature in the vicinity of 20° C., the medium is poured onto 200 ml of water and ice. The precipitate thus formed is isolated by filtration on a sintered glass funnel, rinsed 3 times with 10 ml of water and dried. The filtrate is extracted with 4 times 15 ml of dichloromethane and the combined organic extracts are dried over magnesium sulphate, filtered and concentrated to dryness under reduced pressure. The evaporation residue and the solid isolated above are chromatographed, under argon pressure, on silica gel (eluent dichloromethane/methanol 9/1). 65 mg of 6-(imidazo[1,2-a]pyrimidin-3-ylsulphanyl)-1,3-benzothiazol-2-amine are thus obtained in the form of a light brown solid.

Melting point>260° C. (Köfler).

MS: method A; [M+H]⁺: m/z=300; Tr=0.41 min.

¹H NMR (400 MHz, d₆-DMSO) δ ppm 7.12 (dd, J=8.4, 2.2 Hz, 1H) 7.19 (dd, J=6.8, 4.2 Hz, 1H) 7.23 (d, J=8.4 Hz, 1H) 7.51 (broad s, 2H) 7.60 (d, J=2.2 Hz, 1H) 8.19 (s, 1H) 8.67 (dd, J=4.2, 2.0 Hz, 1H) 8.89 (dd, J=6.8, 2.0 Hz, 1H)

### EXAMPLE 1b

### 1-[2-(morpholin-4-yl)ethyl]-3-(6-sulphanyl-1,3-benzothiazol-2-yl)urea

The compound can be prepared in the following way:

A solution of 11 mg of potassium dihydrogenphosphate in 2.3 ml of water is added to a suspension of 900 mg of 2-({[2-(morpholin-4-yl)ethyl]carbamoyl}amino)-1,3-benzothiazol-6-yl thiocyanate in 35 ml of ethanol at 20° C., followed by 1.1 g of DL-dithiothreitol. The white suspension is stirred at reflux for 18 h. The reaction mixture is cooled to 20° C., 30 ml of water are then added and the mixture is stirred for 15 minutes. The precipitate formed is filtered off and then washed with large amounts of water. 633 mg of 1-[2-(morpholin-4-yl)ethyl]-3-(6-sulphanyl-1,3-benzothiazol-2-yl)urea are thus obtained, in the form of a white solid.

MS: method B; [M+H]⁺: m/z=339; [M−H]⁻: m/z=337; Tr=2.31 min.

### EXAMPLE 1c

### 2-({[2-(morpholin-4-yl)ethyl]carbamoyl}amino)-1,3-benzothiazol-6-yl thiocyanate

The compound can be prepared in the following way:

0.44 ml of 2-(morpholin-4-yl)ethanamine is added at 20° C. to a solution of 1 g of phenyl(6-thiocyanato-1,3-benzothiazol-2-yl)carbamate in 30 ml of tetrahydrofuran. The reaction medium is kept stirred at 20° C. for 24 hours and then concentrated by evaporation under reduced pressure. The residue obtained is chromatographed on a Merck 70 g cartridge (solid deposit; elution with a gradient of dichloromethane and then dichloromethane/methanol 90/10). 902 mg of 2-({[2-(morpholin-4-yl)ethyl]carbamoyl}amino)-1,3-benzothiazol-6-yl thiocyanate are thus obtained in the form of a colourless foam.

MS: method A; [M+H]⁺: m/z=364; Tr=0.99 min.

### EXAMPLE 1d

### phenyl(6-thiocyanato-1,3-benzothiazol-2-yl)carbamate

The compound can be prepared in the following way:

7.5 g of phenyl chlorocarbonate are added, at 20° C., to a solution of 2.5 g of 2-amino-1,3-benzothiazol-6-yl thiocyanate (commercially available product) in 94 ml of tetrahydrofuran, followed by 4.05 g of sodium hydrogencarbonate and 9.4 ml of water. The reaction medium is stirred at 20° C. for 20 hours and then extracted with 2 times 150 ml of ethyl acetate. The organic phases are combined and then washed 3 times with 50 ml of a saturated aqueous sodium hydrogencarbonate solution. The organic phase obtained is dried over magnesium sulphate and then concentrated to dryness under reduced pressure. The residue thus obtained is taken up in 50 ml of water, then filtered off and dried under vacuum at 20° C. 3.45 g of phenyl(6-thiocyanato-1,3-benzothiazol-2-yl)carbamate are thus obtained in the form of a pale yellow solid.

MS: method B; [M+H]⁺: m/z=328; [M−H]⁻: m/z=326; Tr=3.89 min.

The compound 6-(imidazo[1,2-a]pyrimidin-3-ylsulphanyl)-1,3-benzothiazol-2-amine (Example 1 and 1a) can also be obtained in the following way:

A suspension of 310 mg of 4-(imidazo[1,2-a]pyrimidin-3-ylsulphanyl)aniline, 25 ml of acetic acid and 500 mg of potassium thiocyanate is stirred until dissolution is achieved. 66 μl of bromine in solution in 3 ml of acetic acid are subsequently added dropwise. The reaction medium is kept stirred at a temperature in the vicinity of 20° C. for 48 hours and then poured onto 70 ml of ice-cold water. The pH is brought to approximately 11 by addition of 10N sodium hydroxide solution. The precipitate formed is filtered off, washed with water, superficially freed from the washing medium and dried. 242 mg of 6-(imidazo[1,2-a]pyrimidin-3-ylsulphanyl)-1,3-benzothiazol-2-amine are thus obtained in the form of a yellow solid.

### EXAMPLE 1e

### 4-(imidazo[1,2-a]pyrimidin-3-ylsulphanyl)aniline

The compound can be prepared in the following way:

A solution of 770 mg of N-[4-(imidazo[1,2-a]pyrimidin-3-ylsulphanyl)phenyl]acetamide (770 mg; 2.7 mmol), 5.2 ml of hydrochloric acid (37% by volume) and 60 ml of ethanol is brought to reflux for 8 hours. After returning to ambient temperature, the medium is concentrated to dryness by evaporation under reduced pressure and the residue obtained is taken up in a saturated aqueous sodium hydrogencarbonate solution and extracted with 3 times 50 ml of dichloromethane. The combined organic extracts are dried, filtered and concentrated to dryness under reduced pressure. The evaporation residue is chromatographed, under argon pressure, on silica gel (eluent: dichloromethane/methanol 94/6). 480 mg of 4-(imidazo[1,2-a]pyrimidin-3-ylsulphanyl)aniline are thus obtained in the form of a yellow solid.

MS: method A; [M+H]⁺: m/z=243; Tr=0.35 min.

### EXAMPLE 1f

### N-[4-(imidazo[1,2-a]pyrimidin-3-ylsulphanyl)phenyl]acetamide

The compound can be prepared in the following way:

1.42 g of 3-bromoimidazo[1,2-a]pyrimidine (commercially available product), 1.18 g of N-(4-sulphanylphenyl)acetamide (commercially available product), 1.95 g of potassium carbonate and 15 ml of dimethyl sulphoxide are charged to a sealed glass tube. The medium is heated at 180° C. for 12 minutes using microwave radiation. After returning to a temperature in the vicinity of 20° C., the medium is poured onto 250 ml of water and ice. The precipitate thus formed is filtered off, washed with 3 times 70 ml of water and dried, and the filtrate is extracted with 150 ml of dichloromethane. The combined organic extracts are washed with 2 times 30 ml of water, dried over magnesium sulphate, filtered and concentrated to dryness by evaporation under reduced pressure. The precipitate isolated above and the extract are combined in order to be chromatographed on silica gel, under argon pressure (eluent dichloromethane/methanol 9/1). 780 mg of N-[4-(imidazo[1,2-a]pyrimidin-3-ylsulphanyl)phenyl]acetamide are thus obtained in the form of a beige solid.

MS: method A; [M+H]⁺: m/z=285; [M−H]⁻: m/z=283; Tr=1.07 min.

### EXAMPLE 2

### N-[6-(imidazo[1,2-a]pyrimidin-3-ylsulphanyl)-1,3-benzothiazol-2-yl]cyclopropanecarboxamide

The compound can be prepared in the following way:

45 μl of cyclopropanecarbonyl chloride are added dropwise to a solution of 135 mg of 6-(imidazo[1,2-a]pyrimidin-3-ylsulphanyl)-1,3-benzothiazol-2-amine and 5 ml of pyridine. The reaction medium is stirred at a temperature in the vicinity of 20° C. for 16 hours and then concentrated to dryness under reduced pressure. The evaporation residue is chromatographed, under argon pressure, on silica gel (eluent dichloromethane/methanol 94/6). The solid obtained is triturated from ethyl acetate, filtered off and dried. 28 mg of N-[6-(imidazo[1,2-a]pyrimidin-3-ylsulphanyl)-1,3-benzothiazol-2-yl]cyclopropanecarboxamide are thus obtained in the form of a yellow solid.

Melting point=258° C. (Köfler).

MS: method B; [M+H]⁺: m/z=368; [M−H]⁻: m/z=366; Tr=3.23 min.

¹H NMR (400 MHz, d₆-DMSO) δ ppm 0.86-0.96 (m, 4H) 1.91-2.00 (m, 1H) 7.15-7.25 (m, 2H) 7.61 (d, J=8.3 Hz, 1H) 7.82 (d, J=2.0 Hz, 1H) 8.23 (s, 1H) 8.69 (dd, J=4.2, 2.0 Hz, 1H) 8.87 (dd, J=6.8, 2.0 Hz, 1H) 12.54-12.68 (broad unresolved m, 1H)

### EXAMPLE 3

### N-[6-(imidazo[1,2-a]pyrimidin-3-ylsulphanyl)-1,3-benzothiazol-2-yl]acetamide

The compound can be prepared in the following way:

A solution of 73 mg of 6-(imidazo[1,2-a]pyrimidin-3-ylsulphanyl)-1,3-benzothiazol-2-amine, 2 ml of acetic anhydride and 2 ml of pyridine is brought to reflux for 8 hours.

After concentrating the reaction medium to dryness by evaporation under reduced pressure, the residue obtained is chromatographed, under argon pressure, on silica gel (eluent dichloromethane/methanol 95/5). The solid obtained is triturated from 2 ml of isopropanol. The solid obtained is filtered off, washed twice with 1 ml of isopropanol and 3 times with 3 ml of diisopropyl ether, and dried. 51 mg of N-[6-(imidazo[1,2-a]pyrimidin-3-ylsulphanyl)-1,3-benzothiazol-2-yl]acetamide are thus obtained in the form of a yellow solid.

Melting point>260° C. (Köfler).

MS: method A; [M+H]⁺: m/z=342; [M−H]⁻: m/z=340; Tr=0.58 min.

¹H NMR (400 MHz, d₆-DMSO) δ ppm 2.17 (s, 3H) 7.17-7.21 (m, 2H) 7.62 (d, J=8.6 Hz, 1H) 7.83 (d, J=2.0 Hz, 1H) 8.23 (s, 1H) 8.69 (dd, J=4.2, 2.0 Hz, 1H) 8.86 (dd, J=6.7, 2.0 Hz, 1H) 12.25-12.35 (broad unresolved m, 1H)

### EXAMPLE 4

### 1-[6-(imidazo[1,2-a]pyrimidin-3-ylsulphanyl)-1,3-benzothiazol-2-yl]-3-[2-(morpholin-4-yl)ethyl]urea

The compound can be prepared in the following way:

A mixture of 171 mg of 1-[2-(morpholin-4-yl)ethyl]-3-(6-sulphanyl-1,3-benzothiazol-2-yl)urea, 5 ml of ethanol, 1 mg of potassium dihydrogenphosphate, 0.1 ml of water, 100 mg of 3-bromoimidazo[1,2-a]pyrimidine (commercially available product) and 0.1 ml of triethylamine is brought to reflux for 16 hours. The precipitate which appears is removed by filtration on a sintered glass funnel and washed with ethanol, and the filtrate is concentrated to dryness under reduced pressure. The residue isolated is chromatographed, under argon pressure, on silica gel (eluent dichloromethane/methanol 9/1). 22 mg of 1-[6-(imidazo[1,2-a]pyrimidin-3-ylsulphanyl)-1,3-benzothiazol-2-yl]-3-[2-(morpholin-4-yl)ethyl]urea are thus obtained in the form of a white solid.

Melting point>260° C. (Köfler).

MS: method A; [M+H]⁺: m/z=456; [M+H—C₇H₁₂N₂O₂]⁺: m/z=300; Tr=0.45 min.

¹H NMR (400 MHz, d₆-DMSO) δ ppm 2.33-2.45 (m, 6H) 3.25 (partially masked m, 2H) 3.57 (m, 4H) 6.77 (broad m, 1H) 7.13-7.21 (m, 2H) 7.48 (broad d, J=8.8 Hz, 1H) 7.78 (broad s, 1H) 8.22 (s, 1H) 8.67 (dd, J=4.5, 2.1 Hz, 1H) 8.88 (dd, J=6.7, 2.1 Hz, 1H) 10.86 (broad unresolved m, 1H).

### EXAMPLE 5

### 1-[6-(imidazo[1,2-a]pyrimidin-3-ylsulphanyl)-1,3-benzothiazol-2-yl]-3-[2-(pyrrolidin-1-yl)ethyl]urea

### EXAMPLE 5a

### 1-[6-(imidazo[1,2-a]pyrimidin-3-ylsulphanyl)-1,3-benzothiazol-2-yl]-3-[2-(pyrrolidin-1-yl)ethyl]urea

The compound can be prepared in the following way:

0.15 ml of 2-(pyrrolidin-1-yl)ethanamine is added to a suspension of 0.46 g of phenyl [6-(imidazo[1,2-a]pyrimidin-3-ylsulphanyl)-1,3-benzothiazol-2-yl]carbamate in 25 ml of tetrahydrofuran. After stirring at a temperature in the vicinity of 20° C. for 4 hours, 0.015 ml of 2-(pyrrolidin-1-yl)ethanamine is added and the reaction mixture is stirred at a temperature in the vicinity of 20° C. for 2 hours, then at 50° C. for one hour and then at a temperature in the vicinity of 20° C. for 64 hours. The mixture is then cooled using an ice bath and is kept stirred for one hour. The precipitate formed is filtered off on a sintered glass funnel and washed with 10 ml of tetrahydrofuran and with 2 times 10 ml of diethyl ether. The solid isolated is chromatographed, under argon pressure, on silica gel (eluent dichloromethane/methanol/NH₄OH 90/10/0.5). 0.3 g of 1-[6-(imidazo[1,2-a]pyrimidin-3-ylsulphanyl)-1,3-benzothiazol-2-yl]-3-[2-(pyrrolidin-1-yl)ethyl]urea is thus obtained in the form of a white solid.

Melting point>260° C. (Köfler bench).

MS: method A; [M+H]⁺: m/z=440; [M−H]⁻: m/z=438; Tr=0.46 min.

¹H NMR (400 MHz, d₆-DMSO) δ ppm 1.69 (br. s., 4H) 2.42-2.48 (m, 6H) 3.20-3.27 (m, 2H) 6.8 (br. s., 1H) 7.12-7.25 (m, 2H) 7.49 (d, J=8.0 Hz, 1H) 7.78 (d, J=1.5 Hz, 1H) 8.22 (s, 1H) 8.68 (dd, J=4.3, 1.8 Hz, 1H) 8.88 (dd, J=7.0, 1.8 Hz, 1H) 10.71 (br. s., 1H).

### EXAMPLE 5b

### phenyl [6-(imidazo[1,2-a]pyrimidin-3-ylsulphanyl)-1,3-benzothiazol-2-yl]carbamate

The compound can be prepared in the following way:

0.13 ml of phenyl chlorocarbonate is added to a suspension of 0.3 g of 6-(imidazo[1,2-a]pyrimidin-3-ylsulphanyl)-1,3-benzothiazol-2-amine in 5 ml of pyridine. The mixture is stirred at a temperature in the vicinity of 20° C. for 2 hours and then a further 0.13 ml of phenyl chlorocarbonate is added. After stirring for one hour at a temperature in the vicinity of 20° C., the reaction medium is cooled using an ice bath and 20 ml of water are added. After stirring at ambient temperature for two days, the precipitate formed is filtered off on a sintered glass funnel, washed with 3 times 10 ml of water and dried. 0.46 g of phenyl [6-(imidazo[1,2-a]pyrimidin-3-ylsulphanyl)-1,3-benzothiazol-2-yl]carbamate is thus obtained in the form of a yellow solid.

Melting point>260° C. (Köfler bench).

MS: method A; [M+H]⁺: m/z=420; [M−H]⁻: m/z=418; Tr=0.84 min.

### EXAMPLE 6

### N-[6-(imidazo[1,2-a]pyrimidin-3-ylsulphinyl)-1,3-benzothiazol-2-yl]cyclopropanecarboxamide

The compound can be prepared in the following way:

30 mg of 3-chloroperbenzoic acid are added to a heterogeneous solution of 49 mg of N-[6-(imidazo[1,2-a]pyrimidin-3-ylsulphanyl)-1,3-benzothiazol-2-yl]cyclopropanecarboxamide and 5 ml of dichloromethane, and the reaction medium is stirred at a temperature in the vicinity of 20° C. for 96 h. The medium is subsequently diluted with 10 ml of dichloromethane and 10 ml of a saturated aqueous sodium hydrogencarbonate solution. After stirring for 10 minutes, the aqueous phase is separated and extracted with two times 10 ml of dichloromethane. The combined organic extracts are washed with 15 ml of distilled water, dried over magnesium sulphate, filtered and concentrated to dryness under reduced pressure. The evaporation residue is chromatographed on silica gel, under argon pressure (eluent: dichloromethane/methanol 96/4). 6.5 mg of N-[6-(imidazo[1,2-a]pyrimidin-3-ylsulphinyl)-1,3-benzothiazol-2-yl]cyclopropanecarboxamide are thus obtained in the form of a white solid.

Melting point>260° C. (Köfler bench).

MS: method A; [M+H]⁺: m/z=384; [M−H]⁻: m/z=382; Tr=0.55 min.

¹H NMR (400 MHz, d₆-DMSO) δ ppm 0.93 (d, J=5.6 Hz, 2H) 1.95 (br. s., 1H) 7.17 (dd, J=6.9, 4.2 Hz, 1H) 7.61 (d, J=8.5 Hz, 1H) 7.80 (d, J=8.5 Hz, 1H) 8.29 (s, 1H) 8.40 (br. s., 1H) 8.72 (dd, J=4.2, 2 Hz, 1H) 8.86 (dd, J=6.9, 2 Hz, 1H) 12.7 (br. s., 1H)

### EXAMPLE 7

### N-[6-(imidazo[1,2-a]pyrimidin-3-ylsulphonyl)-1,3-benzothiazol-2-yl]cyclopropanecarboxamide

The compound can be prepared in the following way:

270 mg of 3-chloroperbenzoic acid are added to a heterogeneous solution of 200 mg of N-[6-(imidazo[1,2-a]pyrimidin-3-ylsulphanyl)-1,3-benzothiazol-2-yl]cyclopropanecarboxamide and 20 ml of dichloromethane, and the reaction medium is stirred at a temperature in the vicinity of 20° C. for 24 hours. The medium is subsequently taken up in 25 ml of a saturated aqueous sodium hydrogencarbonate solution. After stirring for 15 minutes, the organic phase is filtered, in order to separate a solid, and the filtrate is extracted with 15 ml of dichloromethane. The combined organic extracts are washed with 20 ml of distilled water, dried over magnesium sulphate, filtered and concentrated to dryness under reduced pressure. The evaporation residue and the isolated solid are combined and chromatographed on silica gel, under argon pressure (eluent: dichloromethane/methanol 96/4). The solid isolated is taken up in 2 ml of isopropyl ether, filtered off and dried under reduced pressure. 145 mg of N-[6-(imidazo[1,2-a]pyrimidin-3-ylsulphonyl)-1,3-benzothiazol-2-yl]cyclopropanecarboxamide are thus obtained in the form of a beige solid.

Melting point=232° C. (Köfler bench).

MS: method A; [M+H]⁺: m/z=400; [M−H]⁻: m/z=398; Tr=0.68 min.

¹H NMR (400 MHz, d₆-DMSO) δ ppm 0.93 (d, J=5.6 Hz, 2H) 1.95 (br. s., 1H) 7.17 (dd, J=6.9, 4.2 Hz, 1H) 7.61 (d, J=8.3 Hz, 1H) 7.80 (d, J=8.8 Hz, 1H) 8.29 (s, 1H) 8.40 (br. s., 1H) 8.72 (dd, J=4.3, 2.0 Hz, 1H) 8.86 (dd, J=6.8, 2.0 Hz, 1H).

### EXAMPLE 8

### N-[6-(imidazo[1,2-a]pyrimidin-3-ylsulphanyl)-1,3-benzothiazol-2-yl]-3-(pyrrolidin-1-yl)propanamide

The compound can be prepared in the following way:

A suspension of 0.3 g of 6-(imidazo[1,2-a]pyrimidin-3-ylsulphanyl)-1,3-benzothiazol-2-amine, 1.8 g of 3-(pyrrolidin-1-yl)propionic acid hydrochloride, 1.92 g of 1-(3-dimethylaminopropyl)-3-ethylcarbodiimide hydrochloride and 20 ml of pyridine is kept stirred at a temperature in the vicinity of 20° C. for 3 days. The medium is subsequently brought to 50° C. for 3 hours and 1 g of 1-(3-dimethylaminopropyl)-3-ethylcarbodiimide hydrochloride is added. After stirring at a temperature in the vicinity of 20° C. for 18 hours, 50 ml of water and 150 ml of ethyl acetate are added to the reaction medium. The two phases are combined and concentrated by evaporation under reduced pressure. The residue thus obtained is chromatographed on silica gel, under argon pressure (eluent: dichloromethane/methanol/NH₄OH 95/5/0.5). The solid isolated is again chromatographed on silica gel, under argon pressure (eluent: ethyl acetate/methanol 9/1). The solid isolated is taken up in 20 ml of isopropyl ether, filtered off, washed 3 times with 10 ml of isopropyl ether and then dried. 220 mg of N-[6-(imidazo[1,2-a]pyrimidin-3-ylsulphanyl)-1,3-benzothiazol-2-yl]-3-(pyrrolidin-1-yl)propanamide are thus obtained in the form of a yellow solid.

Melting point=247° C. (Köfler bench).

MS: method B; [M+H]⁺: m/z=425; [M−H]⁻: m/z=423; Tr=2.36 min.

¹H NMR (400 MHz, d₆-DMSO) δ ppm 1.61-1.71 (m, 4H) 2.43-2.48 (m, 4H) 2.63 (d, J=6.3 Hz, 2H) 2.74 (d, J=6.3 Hz, 2H) 7.19 (dd, J=6.8, 4.1 Hz, 1H) 7.22 (dd, J=8.5, 2.0 Hz, 1H) 7.63 (d, J=8.5 Hz, 1H) 7.84 (d, J=2.0 Hz, 1H) 8.24 (s, 1H) 8.69 (dd, J=4.1, 2.0 Hz, 1H) 8.87 (dd, J=6.8, 2.0 Hz, 1H).

### EXAMPLE 9

### N-[6-(imidazo[1,2-a]pyrimidin-3-ylsulphanyl)-1,3-benzothiazol-2-yl]benzamide

The compound can be prepared as in Example 2 but starting from 0.3 g of 6-(imidazo[1,2-a]pyrimidin-3-ylsulphanyl)-1,3-benzothiazol-2-amine, 0.28 g of benzoyl chloride and 5 ml of pyridine. 270 mg of N-[6-(imidazo[1,2-a]pyrimidin-3-ylsulphanyl)-1,3-benzothiazol-2-yl]benzamide are thus obtained in the form of a yellow solid.

Melting point>260° C. (Köfler bench)

MS: method A; [M+H]⁺: m/z=404; [M−H]⁻: m/z=402; Tr=0.83 min.

¹H NMR (400 MHz, d₆-DMSO) δ ppm 7.21 (dd, J=4.2 and 6.8 Hz, 1H); 7.27 (dd, J=2.0 and 8.6 Hz, 1H); 7.55 (t, J=7.8 Hz, 2H); 7.61 to 7.71 (m, 2H); 7.89 (d, J=2.0 Hz, 1H); 8.11 (broad d, J=7.8 Hz, 2H); 8.25 (s, 1H); 8.70 (dd, J=2.2 and 4.2 Hz, 1H); 8.88 (dd, J=2.2 and 6.8 Hz, 1H); 12.87 (broad s, 1H).

### EXAMPLE 10

### N-[6-(imidazo[1,2-a]pyrimidin-3-ylsulphanyl)-1,3-benzothiazol-2-yl]-2-(4-methylpiperazin-1-yl)acetamide

The compound can be prepared as in Example 8 but starting from 85 mg of 6-(imidazo[1,2-a]pyrimidin-3-ylsulphanyl)-1,3-benzothiazol-2-amine, 0.55 g of (4-methylpiperazin-1-yl)acetic acid hydrochloride, 0.54 g of 1-(3-dimethylaminopropyl)-3-ethylcarbodiimide hydrochloride and 3 ml of pyridine. 65 mg of N-[6-(imidazo[1,2-a]pyrimidin-3-ylsulphanyl)-1,3-benzothiazol-2-yl]-2-(4-methylpiperazin-1-yl)acetamide are thus obtained in the form of a crystallizing orange oil.

MS: method B; [M+H]⁺: m/z=440; [M−H]⁻: m/z=438; Tr=2.34 min.

¹H NMR (400 MHz, d₆-DMSO) δ ppm 2.15 (s, 3H); 2.25 to 2.58 (partially masked m, 8H); 3.23 to 3.38 (partially masked m, 2H); 7.19 (dd, J=4.2 and 6.8 Hz, 1H); 7.23 (dd, J=2.1 and 8.4 Hz, 1H); 7.64 (d, J=8.4 Hz, 1H); 7.85 (d, J=2.1 Hz, 1H); 8.24 (s, 1H); 8.69 (dd, J=2.0 and 4.2 Hz, 1H); 8.86 (dd, J=2.0 and 6.8 Hz, 1H); 11.10 to 13.03 (broad unresolved m, 1H).

(4-Methylpiperazin-1-yl)acetic acid can be prepared as described in Patent US 2005/0256164, p. 27.

### EXAMPLE 11

### 2-methylpropan-2-yl(2-{[6-(imidazo[1,2-a]pyrimidin-3-ylsulphanyl)-1,3-benzothiazol-2-yl]amino}-2-oxoethyl)carbamate

The compound can be prepared as in Example 8 but starting from 600 mg of 6-(imidazo[1,2-a]pyrimidin-3-ylsulphanyl)-1,3-benzothiazol-2-amine, 3.5 g of tert-butoxycarbonylaminoacetic acid, 3.83 g of 1-(3-dimethylaminopropyl)-3-ethylcarbodiimide hydrochloride and 30 ml of anhydrous pyridine. 200 mg of 2-methylpropan-2-yl(2-{[6-(imidazo[1,2-a]pyrimidin-3-ylsulphanyl)-1,3-benzothiazol-2-yl]amino}-2-oxoethyl)carbamate are obtained in the form of a cream solid.

Melting point>260° C. (Köfler bench)

MS: method B; [M+H]⁺: m/z=457; [M−H]⁻: m/z=455; Tr=3.46 min.

¹H NMR (400 MHz, d₆-DMSO) δ ppm 1.39 (s, 9H); 3.87 (d, J=6.0 Hz, 2H); 7.13 (broad t, J=6.0 Hz, 1H); 7.19 (dd, J=4.2 and 6.8 Hz, 1H); 7.22 (dd, J=2.0 and 8.6 Hz, 1H); 7.64 (d, J=8.6 Hz, 1H); 7.84 (d, J=2.0 Hz, 1H); 8.24 (s, 1H); 8.69 (dd, J=2.0 and 4.2 Hz, 1H); 8.87 (dd, J=2.0 and 6.8 Hz, 1H); 12.36 (broad s, 1H).

### EXAMPLE 12

### N-[6-(imidazo[1,2-a]pyrimidin-3-ylsulphanyl)-1,3-benzothiazol-2-yl]glycinamide dihydrochloride

A heterogeneous solution of 390 mg of 2-methylpropan-2-yl(2-{[6-(imidazo[1,2-a]pyrimidin-3-ylsulphanyl)-1,3-benzothiazol-2-yl]amino}-2-oxoethyl)carbamate and 21.5 ml of hydrochloric acid in ethyl ether (1M solution) is stirred at a temperature in the vicinity of 20° C. for 4 hours. The reaction medium is subsequently evaporated to dryness under reduced pressure and the evaporation residue is triturated from 10 ml of ethyl acetate before being filtered off, washed with 5 ml of ethyl acetate and then 2 times 5 ml of ethyl ether, superficially freed from the washing medium and dried under reduced pressure. 361 mg of N-[6-(imidazo[1,2-a]pyrimidin-3-ylsulphanyl)-1,3-benzothiazol-2-yl]glycinamide dihydrochloride are thus obtained in the form of a pale yellow solid.

Melting point˜242° C. (Köfler bench).

MS: method A; [M+H]⁺: m/z=357; [M−H]⁻: m/z=355; Tr=0.39 min.

¹H NMR (400 MHz, d₆-DMSO) δ ppm 3.94 (q, J=6.0 Hz, 2H); 7.31 to 7.37 (m, 2H); 7.71 (d, J=8.6 Hz, 1H); 7.87 (d, J=2.0 Hz, 1H); 8.32 (broad t, J=6.0 Hz, 3H); 8.43 (s, 1H); 8.82 (dd, J=2.0 and 4.2 Hz, 1H); 8.98 (dd, J=2.0 and 6.8 Hz, 1H); 12.84 (broad s, 1H).

### EXAMPLE 13

### (trans-A)-N-[6-(imidazo[1,2-a]pyrimidin-3-ylsulphanyl)-1,3-benzothiazol-2-yl]-2-(morpholin-4-ylmethyl)cyclopropanecarboxamide and

### EXAMPLE 14

### (trans-B)-N-[6-(imidazo[1,2-a]pyrimidin-3-ylsulphanyl)-1,3-benzothiazol-2-yl]-2-(morpholin-4-ylmethyl)cyclopropanecarboxamide

The compounds can be prepared as in Example 8 but starting from 300 mg of 6-(imidazo[1,2-a]pyrimidin-3-ylsulphanyl)-1,3-benzothiazol-2-amine, 3 g of (RR,SS)-trans-2-(morpholin-4-ylmethyl)-1-cyclopropanecarboxylic acid, 2.59 g of 1-(3-dimethylaminopropyl)-3-ethylcarbodiimide hydrochloride and 20 ml of anhydrous pyridine. 195 mg of a yellow powder are thus obtained. The two isomers (trans A and trans B) were separated by chromatography (Chiralpak IC 5 μm, eluent: acetonitrile/ethanol/methanol 8/1/1 and then acetonitrile/ethanol/methanol 6/2/2). 47.5 mg of (trans-A)-N-[6-(imidazo[1,2-a]pyrimidin-3-ylsulphanyl)-1,3-benzothiazol-2-yl]-2-(morpholin-4-ylmethyl)cyclopropanecarboxamide are thus obtained in the form of a white solid and 52.3 mg of (trans-B)-N-[6-(imidazo[1,2-a]pyrimidin-3-ylsulphanyl)-1,3-benzothiazol-2-yl]-2-(morpholin-4-ylmethyl)cyclopropanecarboxamide are thus obtained in the form of a white solid.

(trans-A)-N-[6-(imidazo[1,2-a]pyrimidin-3-ylsulphanyl)-1,3-benzothiazol-2-yl]-2-(morpholin-4-ylmethyl)cyclopropanecarboxamide:

MS: method B; [M+H]⁺: m/z=467; [M−H]⁻: m/z=465; Tr=2.54 min.

¹H NMR (400 MHz, d₆-DMSO) δ ppm 0.84 to 0.91 (m, 1H); 1.13 to 1.18 (m, 1H); 1.43 to 1.49 (m, 1H); 1.83 (m, 1H); 2.27 (dd, J=7.3 and 13.0 Hz, 1H); 2.37 (dd, J=6.4 and 13.0 Hz, 1H); 2.41 (m, 4H); 3.57 (m, 4H); 7.20 (dd, J=4.2 and 6.8 Hz, 1H); 7.23 (dd, J=2.2 and 8.5 Hz, 1H); 7.63 (d, J=8.5 Hz, 1H); 7.83 (d, J=2.2 Hz, 1H); 8.25 (s, 1H); 8.70 (dd, J=2.0 and 4.2 Hz, 1H); 8.88 (dd, J=2.0 and 6.8 Hz, 1H); 12.62 (broad s, 1H).

(trans-B)-N-[6-(imidazo[1,2-a]pyrimidin-3-ylsulphanyl)-1,3-benzothiazol-2-yl]-2-(morpholin-4-ylmethyl)cyclopropanecarboxamide:

MS: method B; [M+H]⁺: m/z=467; [M−H]⁻: m/z=465; Tr=2.56 min.

¹H NMR (400 MHz, d₆-DMSO) δ ppm 0.85 to 0.91 (m, 1H); 1.13 to 1.18 (m, 1H); 1.43 to 1.49 (m, 1H); 1.83 (m, 1H); 2.27 (dd, J=6.9 and 12.7 Hz, 1H); 2.37 (dd, J=6.4 and 12.7 Hz, 1H); 2.41 (m, 4H); 3.57 (m, 4H); 7.20 (dd, J=4.4 and 6.7 Hz, 1H); 7.23 (dd, J=2.2 and 8.5 Hz, 1H); 7.63 (d, J=8.5 Hz, 1H); 7.83 (d, J=2.2 Hz, 1H); 8.25 (s, 1H); 8.70 (dd, J=2.0 and 4.4 Hz, 1H); 8.88 (dd, J=2.0 and 6.7 Hz, 1H); 12.62 (broad s, 1H)

(RR,SS)-trans-2-(Morpholin-4-ylmethyl)-1-cyclopropanecarboxylic acid can be prepared as described in Patent WO 2001/02427, p. 59.

### EXAMPLE 15

### 2-(4-ethylpiperazin-1-yl)-N-[6-(imidazo[1,2-a]pyrimidin-3-ylsulphanyl)-1,3-benzothiazol-2-yl]acetamide

The compound can be prepared as in Example 8 but starting from 300 mg of 6-(imidazo[1,2-a]pyrimidin-3-ylsulphanyl)-1,3-benzothiazol-2-amine, 1.27 g of (4-ethylpiperazin-1-yl)acetic acid hydrobromide, 0.96 g of 1-(3-dimethylaminopropyl)-3-ethylcarbodiimide hydrochloride and 20 ml of anhydrous pyridine. 280 mg of 2-(4-ethylpiperazin-1-yl)-N-[6-(imidazo[1,2-a]pyrimidin-3-ylsulphanyl)-1,3-benzothiazol-2-yl]acetamide are thus obtained in the form of a beige solid.

Melting point=210° C. (Köfler bench)

MS: method B; [M+H]⁺: m/z=454; [M−H]⁻: m/z=452; Tr=2.49 min.

¹H NMR (400 MHz, d₆-DMSO) δ ppm 0.97 (t, J=7.2 Hz, 3H); 2.31 (q, J=7.2 Hz, 2H); 2.38 (broad s, 4H); 2.48 to 2.55 (partially masked m, 4H); 3.26 to 3.33 (partially masked m, 2H); 7.19 (dd, J=4.2 and 6.8 Hz, 1H); 7.23 (dd, J=2.1 and 8.5 Hz, 1H); 7.64 (d, J=8.5 Hz, 1H); 7.85 (d, J=2.1 Hz, 1H); 8.24 (s, 1H); 8.69 (dd, J=2.0 and 4.2 Hz, 1H); 8.86 (dd, J=2.0 and 6.8 Hz, 1H); 12.02 (broad unresolved m, 1H)

(4-Ethylpiperazin-1-yl)acetic acid can be prepared as described in Patent US 2005/0256164 p. 28.

### EXAMPLE 16

### 2-(4-cyclopropylpiperazin-1-yl)-N-[6-(imidazo[1,2-a]pyrimidin-3-ylsulphanyl)-1,3-benzothiazol-2-yl]acetamide

### EXAMPLE 16a

### 2-(4-cyclopropylpiperazin-1-yl)-N-[6-(imidazo[1,2-a]pyrimidin-3-ylsulphanyl)-1,3-benzothiazol-2-yl]acetamide

The compound can be prepared in the following way:

A solution of 1.95 g of potassium carboxylate of (4-cyclopropylpiperazin-1-yl)acetic acid and 17.6 ml of ethereal hydrochloric acid (2N solution in diethyl ether) is stirred at a temperature in the vicinity of 20° C. overnight. After concentrating by evaporating under reduced pressure, the white powder thus obtained is reacted as in Example 8 with 260 mg of 6-(imidazo[1,2-a]pyrimidin-3-ylsulphanyl)-1,3-benzothiazol-2-amine, 1.69 g of 1-(3-dimethylaminopropyl)-3-ethylcarbodiimide hydrochloride and 20 ml of anhydrous pyridine. 280 mg of 2-(4-cyclopropylpiperazin-1-yl)-N-[6-(imidazo[1,2-a]pyrimidin-3-ylsulphanyl)-1,3-benzothiazol-2-yl]acetamide are thus obtained in the form of a beige solid.

Melting point=224° C. (Köfler bench).

MS: method A; [M+H]⁺: m/z=466; [M−H]⁻: m/z=464; Tr=0.49 min.

¹H NMR (400 MHz, d₆-DMSO) δ ppm 0.23 to 0.30 (m, 2H); 0.36 to 0.42 (m, 2H); 1.61 (m, 1H); 2.45 to 2.58 (partially masked m, 8H); 3.26 to 3.33 (partially masked m, 2H); 7.19 (dd, J=4.2 and 6.8 Hz, 1H); 7.23 (dd, J=2.0 and 8.6 Hz, 1H); 7.64 (d, J=8.6 Hz, 1H); 7.85 (d, J=2.0 Hz, 1H); 8.24 (s, 1H); 8.69 (dd, J=2.0 and 4.2 Hz, 1H); 8.86 (dd, J=2.0 and 6.8 Hz, 1H); 11.98 (broad unresolved m, 1H).

### EXAMPLE 16b

### potassium carboxylate of (4-cyclopropylpiperazin-1-yl)acetic acid

The compound can be prepared in the following way:

A solution of 1.39 g of 2-bromoacetic acid and 25 ml of water is cooled using a bath of water and ice. 2 g of 4-cyclopropylpiperazine dihydrochloride (commercially available product) and 2.76 g of potassium carbonate are then added and the reaction medium is kept stirred at a temperature in the vicinity of 20° C. for 2 days. After concentrating the reaction medium by evaporation under reduced pressure, the residue obtained is taken up in 50 ml of toluene and then again concentrated by evaporation under reduced pressure. This operation is repeated twice. The white powder thus obtained is taken up in diethyl ether, filtered off, washed with 3 times 20 ml of diethyl ether and dried. The white powder thus obtained is taken up in 50 ml of ethanol and the resulting suspension is stirred at a temperature in the region of 20° C. and then filtered. The solid residue obtained is washed with 3 times 20 ml of ethanol. The filtrate is concentrated by evaporation under reduced pressure and the solid residue is washed with 50 ml of diethyl ether. 1.95 g of potassium carboxylate of (4-cyclopropylpiperazin-1-yl)acetic acid are thus obtained in the form of a white powder.

MS: method B; [M]⁺: m/z=184; base peak: m/z=185; Tr=0.40 min.

### EXAMPLE 17

### N₂,N₂-diethyl-N-[6-(imidazo[1,2-a]pyrimidin-3-ylsulphanyl)-1,3-benzothiazol-2-yl]glycinamide

The compound can be prepared as in Example 16 but starting from 360 mg of 6-(imidazo[1,2-a]pyrimidin-3-ylsulphanyl)-1,3-benzothiazol-2-amine, sodium carboxylate of N,N-diethylglycine, 12 ml of ethereal hydrochloric acid (2N solution in diethyl ether), 2.3 g of 1-(3-dimethylaminopropyl)-3-ethylcarbodiimide hydrochloride and 30 ml of anhydrous pyridine. 220 mg of N₂,N₂-diethyl-N-[6-(imidazo[1,2-a]pyrimidin-3-ylsulphanyl)-1,3-benzothiazol-2-yl]glycinamide are thus obtained in the form of an orange solid.

Melting point=180° C. (Köfler bench).

MS: method A; [M+H]⁺: m/z=413; [M−H]⁻: m/z=411; Tr=0.46 min.

¹H NMR (400 MHz, d₆-DMSO) δ ppm 0.98 (t, J=7.2 Hz, 6H); 2.61 (q, J=7.2 Hz, 4H); 3.38 (s, 2H); 7.19 (dd, J=4.2 and 6.8 Hz, 1H); 7.23 (dd, J=2.1 and 8.5 Hz, 1H); 7.63 (d, J=8.5 Hz, 1H); 7.84 (d, J=2.1 Hz, 1H); 8.24 (s, 1H); 8.69 (dd, J=2.1 and 4.2 Hz, 1H); 8.86 (dd, J=2.1 and 6.8 Hz, 1H); 11.69 (broad unresolved m, 1H).

### EXAMPLE 18

### N-[5-fluoro-6-(imidazo[1,2-a]pyrimidin-3-ylsulphanyl)-1,3-benzothiazol-2-yl]cyclopropanecarboxamide

### EXAMPLE 18a

### N-[5-fluoro-6-(imidazo[1,2-a]pyrimidin-3-ylsulphanyl)-1,3-benzothiazol-2-yl]cyclopropanecarboxamide

The compound can be prepared in the following way:

352 mg of 3-bromoimidazo[1,2-a]pyrimidine (commercially available product), 476 mg of N-[5-fluoro-6-sulphanyl-1,3-benzothiazol-2-yl]cyclopropanecarboxamide, 490 mg of potassium carbonate and 4 ml of dimethyl sulphoxide are charged to a sealed glass tube. The medium is heated at 185° C. for 12 minutes using microwave radiation. After returning to a temperature in the vicinity of 20° C., the medium is poured onto 100 ml of water and ice. The precipitate thus formed is isolated by filtration on a sintered glass funnel, washed with water and dried. The solid isolated is then partially dissolved in a dichloromethane/methanol (90/10) mixture and filtered through a sintered glass funnel and the filtrate is concentrated to dryness by evaporation under reduced pressure. The solid isolated is chromatographed a first time on silica gel, under argon pressure (eluent dichloromethane/methanol 96/4). The advantageous fractions are concentrated to dryness by evaporation under reduced pressure and then chromatographed a second time, under argon pressure, on silica gel (eluent dichloromethane/methanol 98/2). 121 mg of N-[5-fluoro-6-(imidazo[1,2-a]pyrimidin-3-ylsulphanyl)-1,3-benzothiazol-2-yl]cyclopropanecarboxamide are thus obtained in the form of a beige solid.

Melting point>260° C. (Köfler bench).

MS: method B; [M+H]⁺: m/z=386; [M−H]⁻: m/z=384; Tr=3.35 min.

¹H NMR (400 MHz, d₆-DMSO) δ ppm 0.86-0.99 (m, 4H) 1.91-2.03 (m, 1H) 7.23 (dd, J=6.9, 4.1 Hz, 1H) 7.62 (d, J=10.5 Hz, 1H) 7.68 (d, J=7.6 Hz, 1H) 8.21 (s, 1H) 8.70 (dd, J=4.1, 2.0 Hz, 1H) 8.92 (dd, J=6.9, 2.0 Hz, 1H) 12.67 (br. s., 1H).

### EXAMPLE 18b

### N-[5-fluoro-6-sulphanyl-1,3-benzothiazol-2-yl]cyclopropane-carboxamide

The compound can be prepared in the following way:

555 mg of 2-[(cyclopropylcarbonyl)amino]-5-fluoro-1,3-benzothiazol-6-yl thiocyanate, 18 ml of ethanol, a solution of 25 mg of potassium dihydrogenphosphate in 2 ml of distilled water and 853 mg of 1,4-dithio-DL-threitol are successively charged to a single-necked flask and the heterogeneous solution is brought to reflux for 2 hours. The reaction medium is subsequently poured onto 200 ml of distilled water and stirred for 10 minutes and then the solid is isolated by filtration, washed with water, superficially freed from the washing medium and dried under reduced pressure. 476 mg of N-[5-fluoro-6-sulphanyl-1,3-benzothiazol-2-yl]cyclopropanecarboxamide are obtained in the form of an off-white solid.

MS: method A; [M+H]⁺: m/z=269; [M−H]⁻: m/z=267; Tr=0.91 min.

¹H NMR (400 MHz, d₆-DMSO) δ ppm 0.88-1.02 (m, 4H) 1.93-2.06 (m, 1H) 5.47 (br. s., 1H) 7.59 (d, J=10.3 Hz, 1H) 8.01 (d, J=7.6 Hz, 1H) 12.67 (br. s., 1H).

### EXAMPLE 18c

### 2-[(cyclopropylcarbonyl)amino]-5-fluoro-1,3-benzothiazol-6-yl thiocyanate

The compound can be prepared according to Example 2, starting from 510 mg of 2-amino-5-fluoro-1,3-benzothiazol-6-yl thiocyanate and 262 μl of cyclopropanecarbonyl chloride in 10 ml of pyridine. 556 mg of 2-[(cyclopropylcarbonyl)amino]-5-fluoro-1,3-benzothiazol-6-yl thiocyanate are thus obtained in the form of an off-white solid.

Melting point=256° C. (Köfler bench).

MS: method A; [M+H]⁺: m/z=294; [M−H]⁻: m/z=292; Tr=0.90 min.

¹H NMR (400 MHz, d₆-DMSO) δ ppm 0.89-1.06 (m, 4H) 1.95-2.09 (m, 1H) 7.83 (d, J=10.0 Hz, 1H) 8.47 (d, J=7.1 Hz, 1H) 12.91 (br. s., 1H).

### EXAMPLE 18d

### 2-amino-5-fluoro-1,3-benzothiazol-6-yl thiocyanate

The compound can be prepared in the following way:

3.9 g of potassium thiocyanate are added to a solution of 960 μl of 3-fluoroaniline in 40 ml of acetic acid and the combined mixture is stirred until the former has completely dissolved. A solution of 1.02 ml of bromine and 5 ml of acetic acid is subsequently run in dropwise. The reaction medium is stirred at a temperature in the vicinity of 20° C. for 16 hours. The thick medium is subsequently poured onto 100 ml of water cooled with an ice bath and then basification is carried out to a pH in the vicinity of 10 with a 28% aqueous ammonia solution. The solid formed is isolated by filtration, washed with distilled water, superficially freed from the washing medium and dried under reduced pressure and then chromatographed on silica gel, under argon pressure (eluent dichloromethane/methanol 95/5). The evaporation to dryness under reduced pressure of the fractions makes it possible to obtain 330 mg of 2-amino-5-fluoro-1,3-benzothiazol-6-yl thiocyanate in the form of a yellow solid.

MS: method A; [M+H]⁺: m/z=226; [M−H]⁻: m/z=224; Tr=0.65 min.

¹H NMR (400 MHz, d₆-DMSO) δ ppm 7.36 (d, J=10.8 Hz, 1H) 8.01 (s, 2H) 8.11 (d, J=7.1 Hz, 1H)

### EXAMPLE 19

### 5-fluoro-6-(imidazo[1,2-a]pyrimidin-3-ylsulphanyl)-1,3-benzo-thiazol-2-amine

### EXAMPLE 19a

### 5-fluoro-6-(imidazo[1,2-a]pyrimidin-3-ylsulphanyl)-1,3-benzothiazol-2-amine

The compound can be prepared in the following way:

200 mg of 3-bromoimidazo[1,2-a]pyrimidine (commercially available product), 240 mg of 2-amino-5-fluoro-1,3-benzothiazole-6-thiol, 0.36 ml of N,N-diisopropylethylamine, 170 mg of bis(diphenylphosphino)-9,9-dimethylxanthene, 140 mg of tris(dibenzylideneacetone)dipalladium(0), 2 ml of 1,4-dioxane and two drops of dimethylformamide are charged to a sealed glass tube. The medium is heated at 160° C. for 30 minutes using microwave radiation. After returning to a temperature in the vicinity of 20° C. the medium is concentrated by evaporation under reduced pressure and then chromatographed on silica gel, under argon pressure (eluent dichloromethane/methanol/NH₄OH 95/5/0.5). Methanol and methanolic hydrochloric acid solution are added to the fractions comprising the expected product and then concentrating is carried out by evaporation under reduced pressure. The orange solid thus obtained is taken up in an aqueous potassium carbonate solution and kept stirred. The precipitate formed is filtered off on a sintered glass funnel and washed with 3 times 10 ml of water, 2 times 10 ml of ethanol and 2 times 10 ml of isopropyl ether. The white powder thus obtained is dissolved in 2 ml of dimethyl sulphoxide. After having warmed the suspension until complete dissolution is achieved and then cooling, a slight precipitate is filtered off. 10 ml of water are then added to the filtrate and the white precipitate obtained is filtered off on a sintered glass funnel, washed with 3 times 10 ml of water and 3 times 10 ml of diethyl ether and dried. 53 mg of 5-fluoro-6-(imidazo[1,2-a]pyrimidin-3-ylsulphanyl)-1,3-benzothiazol-2-amine are thus obtained in the form of a white powder.

Melting point>264° C. (Köfler bench).

MS: method A; [M+H]⁺: m/z=318; [M−H]⁻: m/z=316; Tr=0.54 min.

¹H NMR (400 MHz, d₆-DMSO) δ ppm 7.19 (d, J=10.8 Hz, 1H); 7.23 (dd, J=4.3 and 6.7 Hz, 1H); 7.53 (d, J=7.3 Hz, 1H); 7.70 (broad s, 2H); 8.16 (s, 1H); 8.68 (dd, J=2.1 and 4.3 Hz, 1H); 8.93 (dd, J=2.1 and 6.7 Hz, 1H).

### EXAMPLE 19b

### 2-amino-5-fluoro-1,3-benzothiazole-6-thiol

The compound can be prepared as in Example 18b but starting from 1 g of 2-amino-5-fluoro-1,3-benzothiazol-6-yl thiocyanate, 30 ml of ethanol, a solution of 14 mg of potassium dihydrogenphosphate in 3 ml of distilled water and 1.58 g of 1,4-dithio-DL-threitol. 750 mg of 2-amino-5-fluoro-1,3-benzothiazole-6-thiol are thus obtained in the form of a pale yellow solid.

Melting point=223° C. (Köfler bench).

MS: method A; [M+H]⁺: m/z=201; Tr=0.58 min.

### EXAMPLE 20

### N-[6-(imidazo[1,2-a]pyrimidin-3-ylsulphanyl)-1,3-benzothiazol-2-yl]-3-methoxypropanamide

The compound can be prepared as in Example 8 but starting from 320 mg of 6-(imidazo[1,2-a]pyrimidin-3-ylsulphanyl)-1,3-benzothiazol-2-amine, 1 ml of 3-methoxypropanoic acid, 2.05 g of 1-(3-dimethylaminopropyl)-3-ethylcarbodiimide hydrochloride and 20 ml of anhydrous pyridine. 45 mg of N-[6-(imidazo[1,2-a]pyrimidin-3-ylsulphanyl)-1,3-benzothiazol-2-yl]-3-methoxypropanamide are thus obtained in the form of a white solid.

Melting point=225° C. (Köfler bench).

MS: method A; [M+H]⁺ m/z=386; [M−H]⁻ m/z=384; Tr=0.64 min.

¹H NMR (400 MHz, d₆-DMSO) δ ppm 2.70 (t, J=6.1 Hz, 2H); 3.23 (s, 3H); 3.63 (t, J=6.1 Hz, 2H); 7.20 (dd, J=4.3 and 6.8 Hz, 1H); 7.23 (dd, J=2.1 and 8.5 Hz, 1H); 7.63 (d, J=8.5 Hz, 1H); 7.85 (d, J=2.1 Hz, 1H); 8.24 (s, 1H); 8.69 (dd, J=2.1 and 4.3 Hz, 1H); 8.87 (dd, J=2.1 and 6.8 Hz, 1H); 12.35 (broad unresolved m, 1H).

### EXAMPLE 21

### N-[6-(imidazo[1,2-a]pyrimidin-3-ylsulphanyl)-1,3-benzothiazol-2-yl]-2-(4-methyl-3-oxopiperazin-1-yl)acetamide

### EXAMPLE 21a

### N-[6-(imidazo[1,2-a]pyrimidin-3-ylsulphanyl)-1,3-benzothiazol-2-yl]-2-(4-methyl-3-oxopiperazin-1-yl)acetamide

The compound can be prepared as in Example 8 but starting from 140 mg of 6-(imidazo[1,2-a]pyrimidin-3-ylsulphanyl)-1,3-benzothiazol-2-amine, 1 ml of (4-methyl-3-oxopiperazin-1-yl)acetic acid hydrochloride, 0.94 g of 1-(3-dimethylaminopropyl)-3-ethylcarbodiimide hydrochloride and 10 ml of anhydrous pyridine. 160 mg of N-[6-(imidazo[1,2-a]pyrimidin-3-ylsulphanyl)-1,3-benzothiazol-2-yl]-2-(4-methyl-3-oxopiperazin-1-yl)acetamide are thus obtained in the form of a beige solid.

Melting point˜264° C. (Köfler bench).

MS: method B; [M+H]⁺ m/z=454; [M−H]⁻ m/z=452; Tr=2.77 min.

¹H NMR (400 MHz, d₆-DMSO) δ ppm 2.77 to 2.84 (m, 5H); 3.16 (s, 2H); 3.24 to 3.33 (partially masked m, 2H); 3.42 (s, 2H); 7.19 (dd, J=4.3 and 6.8 Hz, 1H); 7.23 (broad d, J=8.5 Hz, 1H); 7.62 (broad d, J=8.5 Hz, 1H); 7.84 (broad s, 1H); 8.24 (s, 1H); 8.69 (dd, J=2.1 and 4.3 Hz, 1H); 8.87 (dd, J=2.1 and 6.8 Hz, 1H); 12.17 (broad unresolved m, 1H).

### EXAMPLE 21b

### (4-methyl-3-oxopiperazin-1-yl)acetic acid

The compound can be prepared in the following way:

A solution of 0.61 g of 2-bromoacetic acid, 10 ml of water, 0.74 g of 1-methyl-piperazin-2-one hydrochloride (commercially available product) and 0.61 g of potassium carbonate is kept stirred at a temperature in the vicinity of 20° C. for 18 hours. 0.31 g of potassium carbonate is then added and stirring is maintained for one hour. The reaction medium is acidified (pH˜1) by addition of an aqueous hydrochloric acid solution (1N) and then concentrated by evaporation under reduced pressure. The residue obtained is taken up in 2 times 30 ml of toluene and then concentrated. The yellow solid obtained is taken up in 5 ml of ethanol, filtered off on a sintered glass funnel and washed with 2 times 5 ml of ethanol. The filtrate is concentrated by evaporation under reduced pressure and 1.03 g of (4-methyl-3-oxopiperazin-1-yl)acetic acid hydrochloride are thus obtained in the form of a yellow foam.

MS: method A; [M+H]⁺: m/z=173; [M−H]⁻: m/z=171; Tr=0.11 min.

### EXAMPLE 22

### N-{6-[(7-aminoimidazo[1,2-a]pyrimidin-3-yl)sulphanyl]-1,3-benzothiazol-2-yl}cyclopropanecarboxamide

### EXAMPLE 22a

### N-{6-[(7-aminoimidazo[1,2-a]pyrimidin-3-yl)sulphanyl]-1,3-benzo-thiazol-2-yl}cyclopropanecarboxamide

The compound can be prepared as in Example 18a but starting from 0.88 g of bis(2-methylpropan-2-yl) (3-bromoimidazo[1,2-a]pyrimidin-7-yl)imidodicarbonate, 640 mg of N-(6-sulphanyl-1,3-benzothiazol-2-yl)cyclopropanecarboxamide, 620 mg of potassium carbonate and 10 ml of dimethyl sulphoxide. 240 mg of N-{6-[(7-aminoimidazo[1,2-a]pyrimidin-3-yl)sulphanyl]-1,3-benzothiazol-2-yl}cyclo-propanecarboxamide are thus obtained in the form of a pale yellow solid.

Melting point>264° C. (Köfler bench).

MS: method A; [M+H]⁺ m/z=383; [M−H]⁻ m/z=381; Tr=0.54 min.

¹H NMR (400 MHz, d₆-DMSO) δ ppm 0.90 to 0.98 (m, 4H); 1.93 to 2.01 (m, 1H); 6.35 (d, J=7.3 Hz, 1H); 7.07 (broad s, 2H); 7.14 (dd, J=2.0 and 8.6 Hz, 1H); 7.63 (d, J=8.6 Hz, 1H); 7.66 (s, 1H); 7.77 (d, J=2.0 Hz, 1H); 8.22 (d, J=7.3 Hz, 1H); 12.63 (broad unresolved m, 1H).

### EXAMPLE 22b

### N-(6-sulphanyl-1,3-benzothiazol-2-yl)cyclopropanecarboxamide

The compound can be prepared in the following way:

A solution of 33.6 mg of potassium dihydrogenphosphate in 8 ml of water is added, at 20° C., to a suspension of 2 g of (6-thiocyanato-1,3-benzothiazol-2-yl)cyclopropanecarboxamide and 70 ml of ethanol, followed by 3.2 g of 1,4-dithio-DL-threitol. The reaction medium is stirred at reflux for 5 hours and then brought back to a temperature in the vicinity of 20° C. 400 ml of water are then added and the precipitate formed is filtered off on a sintered glass funnel, washed copiously with water, superficially freed from the washing medium and then dried. 1.5 g of N-(6-sulphanyl-1,3-benzothiazol-2-yl)cyclopropanecarboxamide are thus obtained in the form of a pale yellow solid.

MS: method B; [M+H]⁺ m/z=251; [M−H]⁻ m/z=249; Tr=3.77 min.

### EXAMPLE 22c

### (6-thiocyanato-1,3-benzothiazol-2-yl)cyclopropanecarboxamide

The compound can be prepared in the following way:

5.3 ml of cyclopropanecarbonyl chloride are added to a solution of 10 g of 2-amino-1,3-benzothiazol-6-yl thiocyanate (commercially available product) and 100 ml of pyridine while maintaining the temperature in the vicinity of 20° C. The reaction medium is stirred for 4 hours and then 500 ml of water are added. The precipitate formed is filtered off on a sintered glass funnel, washed copiously with water, superficially freed from the washing medium and then dried. 13 g of (6-thiocyanato-1,3-benzothiazol-2-yl)cyclopropanecarboxamide are thus obtained in the form of a pale yellow solid used as is in the following stages.

Bis(2-methylpropan-2-yl)(3-bromoimidazo[1,2-a]pyrimidin-7-yl)imidodicarbonate can be prepared as described in Patent WO 2002/074773 p. 62.

### EXAMPLE 23

### N-(6-{[6-(3-fluorophenyl)imidazo[1,2-a]pyrimidin-3-yl]sulphanyl}-1,3-benzothiazol-2-yl)cyclopropanecarboxamide

### EXAMPLE 23a

### N-(6-{[6-(3-fluorophenyl)imidazo[1,2-a]pyrimidin-3-yl]sulphanyl}-1,3-benzothiazol-2-yl)cyclopropanecarboxamide

The compound can be prepared in the following way:

450 mg of 3-bromo-6-(3-fluorophenyl)imidazo[1,2-a]pyrimidine, 400 mg of N-[6-sulphanyl-1,3-benzothiazol-2-yl]cyclopropanecarboxamide, 430 mg of potassium carbonate and 10 ml of dimethyl sulphoxide are charged to a sealed glass tube. The medium is heated at 185° C. for 12 minutes using microwave radiation. After returning to a temperature in the vicinity of 20° C., the medium is poured onto 200 ml of water and ice and extracted with 4 times 50 ml of a dichloromethane/methanol 90/10 mixture. The combined organic extracts are washed with 2 times 50 ml of distilled water, dried over magnesium sulphate, filtered and concentrated to dryness by evaporation under reduced pressure. The solid isolated is chromatographed a first time on silica gel, under argon pressure (eluent dichloromethane/methanol 96/4). The advantageous fractions are concentrated to dryness by evaporation under reduced pressure and then chromatographed a second time on a Chiralpak IC 20 μM column (eluent acetonitrile/ethanol 90/10). 86 mg of N-(6-{[6-(3-fluorophenyl)imidazo[1,2-a]pyrimidin-3-yl]sulphanyl}-1,3-benzothiazol-2-yl)cyclopropanecarboxamide are thus obtained in the form of an ochre solid.

MS: method B; [M+H]⁺ m/z=462; [M−H]⁻ m/z=460; Tr=4.14 min.

¹H NMR (400 MHz, d₆-DMSO) δ ppm 0.89 to 0.96 (m, 4H); 1.91 to 2.01 (m, 1H); 7.24 to 7.33 (m, 2H); 7.55 (dt, J=6.3 and 8.1 Hz, 1H); 7.62 (m, 2H); 7.70 (td, J=2.0 and 10.5 Hz, 1H); 7.89 (d, J=2.0 Hz, 1H); 8.27 (s, 1H); 9.07 (d, J=2.7 Hz, 1H); 9.08 (d, J=2.7 Hz, 1H); 12.57 (broad unresolved m, 1H).

### EXAMPLE 23b

### 3-bromo-6-(3-fluorophenyl)imidazo[1,2-a]pyrimidine

The compound can be prepared in the following way:

A solution of 426 mg of 6-(3-fluorophenyl)imidazo[1,2-a]pyrimidine, 356 mg of N-bromosuccinimide and 20 ml of chloroform is heated at reflux for 5 hours. After concentrating to dryness by evaporation of the reaction medium under reduced pressure, the residue obtained is taken up in 30 ml of distilled water and stirred for 30 minutes and the solid is isolated by filtration, washed with distilled water, then with 5 ml of ethanol and subsequently with 5 ml of ethyl ether, superficially freed from the washing medium and dried under reduced pressure. 450 mg of 3-bromo-6-(3-fluorophenyl)imidazo[1,2-a]pyrimidine are thus obtained in the form of a beige solid.

MS: method A; [M+H]⁺ m/z=292; Tr=0.77 min.

¹H NMR (400 MHz, d₆-DMSO) δ ppm 7.25-7.38 (m, 1H) 7.59 (td, J=8.0, 6.2 Hz, 1H) 7.68-7.76 (m,1H) 7.81 (dt, J=10.5, 2.1 Hz, 1H) 7.95 (s, 1H) 8.95-9.03 (m, 2H).

### EXAMPLE 23c

### 6-(3-fluorophenyl)imidazo[1,2-a]pyrimidine

The compound can be prepared in the following way:

400 mg of 6-bromoimidazo[1,2-a]pyrimidine (commercially available product), 345 mg of 3-fluorophenylboronic acid, 69 mg of tetrakis(triphenylphosphine)palladium, 2 ml of a 2M aqueous sodium carbonate solution and 8 ml of dimethylformamide are charged to a sealed glass tube. The medium is heated at 150° C. for 20 minutes using microwave radiation. After returning to a temperature in the vicinity of 20° C., the medium is filtered through a bed of Clarcel Flo M and rinsing is carried out with 2 times 2 ml of dimethylformamide and then 2 times 5 ml of methanol. The filtrate is concentrated to dryness by evaporation under reduced pressure. The solid isolated is suspended in 80 ml of distilled water, stirred, filtered off, washed with distilled water, superficially freed from the washing medium and dried under reduced pressure. 430 mg of 6-(3-fluorophenyl)imidazo[1,2-a]pyrimidine are thus obtained in the form of a light brown solid.

MS: method A; [M+H]⁺ m/z=214; Tr=0.38 min.

¹H NMR (400 MHz, d₆-DMSO) δ ppm 7.28 (td, J=8.5, 2.6 Hz, 1H) 7.51-7.66 (m, 2H) 7.69 (dt, J=10.5, 2.0 Hz, 1H) 7.78 (d, J=1.5 Hz, 1H) 7.92 (d, J=1.5 Hz, 1H) 8.93 (d, J=2.7 Hz, 1H) 9.38 (d, J=2.7 Hz, 1H).

### EXAMPLE 24

### N-(6-{[6-(cyclohexyloxy)imidazo[1,2-a]pyrimidin-3-yl]sulphanyl}-1,3-benzothiazol-2-yl)cyclopropanecarboxamide

### EXAMPLE 24a

### N-(6-{[6-(cyclohexyloxy)imidazo[1,2-a]pyrimidin-3-yl]sulphanyl}-1,3-benzothiazol-2-yl)cyclopropanecarboxamide

The compound can be prepared as in Example 19a but starting from 78 mg of 3-bromo-6-(cyclohexyloxy)imidazo[1,2-a]pyrimidine, 85 mg of (6-sulphanyl-1,3-benzothiazol-2-yl)cyclopropanecarboxamide, 93 μl of N,N-diisopropylethylamine, 36 mg of tris(dibenzylideneacetone)dipalladium(0), 46 mg of 4,5-bis(diphenyl-phosphino)-9,9-dimethylxanthene and 3 ml of 1,4-dioxane. 44 mg of N-(6-{[6-(cyclohexyloxy)imidazo[1,2-a]pyrimidin-3-yl]sulphanyl}-1,3-benzothiazol-2-yl)cyclopropanecarboxamide are thus obtained in the form of a cream solid.

Melting point˜161° C. (Köfler bench).

MS: method A; [M+H]⁺ m/z=466; [M−H]⁻ m/z=464; Tr=1.01 min.

¹H NMR (400 MHz, d₆-DMSO) δ ppm 0.88 to 0.98 (m, 4H); 1.14 to 1.41 (m, 5H); 1.46 (m, 1H); 1.55 to 1.66 (m, 2H); 1.73 to 1.83 (m, 2H); 1.93 to 2.02 (m, 1H); 4.28 to 4.41 (m, 1H); 7.26 (dd, J=2.0 and 8.6 Hz, 1H); 7.63 (d, J=8.6 Hz, 1H); 7.85 (d, J=2.0 Hz, 1H); 8.13 (s, 1H); 8.26 (d, J=2.9 Hz, 1H); 8.53 (d, J=2.9 Hz, 1H); 12.60 (broad unresolved m, 1H).

### EXAMPLE 24b

### 3-bromo-6-(cyclohexyloxy)imidazo[1,2-a]pyrimidine

3-Bromo-6-(cyclohexyloxy)imidazo[1,2-a]pyrimidine can be prepared as in Example 23b but starting from 74 mg of 6-(cyclohexyloxy)imidazo[1,2-a]pyrimidine, 7 ml of chloroform and 65 mg of N-bromosuccinimide. 79 mg of 3-bromo-6-(cyclohexyloxy)imidazo[1,2-a]pyrimidine are thus obtained in the form of a brown oil.

MS: method B; [M+H]⁺ m/z=296; Tr=3.84 min.

### EXAMPLE 24c

### 6-(cyclohexyloxy)imidazo[1,2-a]pyrimidine

6-(Cyclohexyloxy)imidazo[1,2-a]pyrimidine can be prepared in the following way:

12 ml of ethanol, 920 mg of potassium hydroxide pellets and 1 g of 6-bromoimidazo[1,2-a]pyrimidine are charged to a glass tube. The tube is sealed and heated at 135° C. for 12 minutes using microwave radiation. After returning to a temperature in the vicinity of 20° C., 1.5 ml of bromocyclohexane are added. The tube is again sealed and the combined mixture is heated at 140° C. for 15 minutes using microwave radiation. After returning to a temperature in the vicinity of 20° C., the reaction medium is evaporated to dryness under reduced pressure and the solid isolated is chromatographed, under argon pressure, on silica gel (eluent dichloromethane/methanol 97/3). 75 mg of 6-(cyclohexyloxy)imidazo[1,2-a]pyrimidine are thus obtained in the form of a beige solid.

MS: method B; [M+H]⁺ m/z=218; Tr=2.54 min.

### EXAMPLE 25

### 3-[(2-amino-1,3-benzothiazol-6-yl)sulphanyl]-N-cyclohexyl-imidazo[1,2-a]pyrimidin-6-amine

### EXAMPLE 25a

### 3-[(2-amino-1,3-benzothiazol-6-yl)sulphanyl]-N-cyclohexylimidazo[1,2-a]pyrimidin-6-amine

The compound can be prepared as in Example 19a but starting from 310 mg of 3-bromo-N-cyclohexylimidazo[1,2-a]pyrimidin-6-amine, 230 mg of (6-sulphanyl-1,3-benzothiazol-2-yl)cyclopropanecarboxamide, 380 μl of N,N-diisopropylethylamine, 140 mg of tris(dibenzylideneacetone)dipalladium(0), 180 mg of 4,5-bis(diphenylphosphino)-9,9-dimethylxanthene, 3 ml of 1,4-dioxane and 5 drops of dimethylformamide. 25 mg of 3-[(2-amino-1,3-benzothiazol-6-yl)sulphanyl]-N-cyclohexylimidazo[1,2-a]pyrimidin-6-amine are thus obtained in the form of a beige solid.

MS: method A; [M+H]⁺ m/z=397; [M−H]⁻ m/z=395; Tr=0.68 min.

¹H NMR (400 MHz, d₆-DMSO) δ ppm 0.94 to 1.35 (m, 5H); 1.50 to 1.69 (m, 3H); 1.71 to 1.81 (m, 2H); 2.97 to 3.10 (m, 1H); 5.82 (d, J=7.6 Hz, 1H); 7.08 (dd, J=2.1 and 8.6 Hz, 1H); 7.23 (d, J=8.6 Hz, 1H); 7.51 (broad s, 2H); 7.53 (d, J=2.9 Hz, 1H); 7.58 (d, J=2.1 Hz, 1H); 7.90 (s, 1H); 8.32 (d, J=2.9 Hz, 1H).

### EXAMPLE 25b

### 3-bromo-N-cyclohexylimidazo[1,2-a]pyrimidin-6-amine

The compound can be prepared as in Example 23b but starting from 720 mg of N-cyclohexylimidazo[1,2-a]pyrimidin-6-amine, 60 ml of chloroform and 530 mg of N-bromosuccinimide. 330 mg of 3-bromo-N-cyclohexylimidazo[1,2-a]pyrimidin-6-amine are thus obtained in the form of a brown powder.

Melting point=190° C. (Köfler bench).

MS: method A; [M+H]⁺ m/z=295; Tr=0.72 min.

### EXAMPLE 25c

### N-cyclohexylimidazo[1,2-a]pyrimidin-6-amine

The product can be prepared in the following way:

3.2 g of 6-bromoimidazo[1,2-a]pyrimidine, 5.5 ml of cyclohexylamine and 32 ml of acetonitrile are charged to a glass tube. The tube is sealed and heated at 120° C. for 30 minutes using microwave radiation. After returning to a temperature in the vicinity of 20° C., 100 ml of an aqueous potassium carbonate solution are added and the resulting aqueous phase is extracted with 3 times 150 ml of ethyl acetate and 1 times 150 ml of dichloromethane. The organic phases are combined, washed with 2 times 200 ml of an aqueous sodium chloride solution, dried over sodium sulphate, filtered and concentrated by evaporation under reduced pressure. The residue obtained is chromatographed on silica gel (eluent dichloromethane/methanol 95/5). 720 mg of N-cyclohexylimidazo[1,2-a]pyrimidin-6-amine are thus obtained in the form of a brown oil.

MS: method A; [M+H]⁺ m/z=217; Tr=0.45 min.

### EXAMPLE 26

### N-(6-{[6-(benzylamino)imidazo[1,2-a]pyrimidin-3-yl]sulphanyl}-1,3-benzothiazol-2-yl)cyclopropanecarboxamide

### EXAMPLE 26a

### N-(6-{[6-(benzylamino)imidazo[1,2-a]pyrimidin-3-yl]sulphanyl}-1,3-benzothiazol-2-yl)cyclopropanecarboxamide

N-(6-{[6-(Benzylamino)imidazo[1,2-a]pyrimidin-3-yl]sulphanyl}-1,3-benzothiazol-2-yl)cyclopropanecarboxamide can be prepared as in Example 19a but starting from 100 mg of N-benzyl-3-bromoimidazo[1,2-a]pyrimidin-6-amine, 95 mg of (6-sulphanyl-1,3-benzothiazol-2-yl)cyclopropanecarboxamide, 114 μl of N,N-diisopropylethylamine, 43 mg of tris(dibenzylideneacetone)dipalladium(0), 55 mg of 4,5-bis(diphenylphosphino)-9,9-dimethylxanthene and 4 ml of 1,4-dioxane. 65 mg of N-(6-{[6-(benzylamino)imidazo[1,2-a]pyrimidin-3-yl]sulphanyl}-1,3-benzothiazol-2-yl)cyclopropanecarboxamide are thus obtained in the form of a light yellow solid.

Melting point>260° C. (Köfler bench).

MS: method A; [M+H]⁺ m/z=473; [M−H]⁻ m/z=471; Tr=0.81 min.

¹H NMR (400 MHz, d₆-DMSO) δ ppm 0.92 to 0.97 (m, 4H); 1.94 to 2.03 (m, 1H); 4.20 (d, J=6.0 Hz, 2H); 6.68 (t, J=6.0 Hz, 1H); 7.03 to 7.11 (m, 2H); 7.18 (t, J=7.5 Hz, 2H); 7.27 (d, J=7.5 Hz, 2H); 7.53 to 7.61 (m, 2H); 7.69 (d, J=2.0 Hz, 1H); 7.94 (s, 1H); 8.42 (d, J=2.9 Hz, 1H); 12.62 (broad unresolved m, 1H).

### EXAMPLE 26b

### N-benzyl-3-bromoimidazo[1,2-a]pyrimidin-6-amine

N-Benzyl-3-bromoimidazo[1,2-a]pyrimidin-6-amine can be prepared as in Example 23b but starting from 110 mg of N-benzylimidazo[1,2-a]pyrimidin-6-amine, 10 ml of chloroform and 89 mg of N-bromosuccinimide. 109 mg of N-benzyl-3-bromoimidazo[1,2-a]pyrimidin-6-amine are thus obtained in the form of a beige solid.

MS: method A; [M+H]⁺ m/z=303; Tr=0.66 min.

### EXAMPLE 26c

### N-benzylimidazo[1,2-a]pyrimidin-6-amine

N-Benzylimidazo[1,2-a]pyrimidin-6-amine can be prepared in the following way:

670 μl of benzylamine, 2 ml of acetonitrile and 400 mg of 6-bromoimidazo[1,2-a]pyrimidine are charged to a glass tube. The tube is sealed and heated at 120° C. for 30 minutes using microwave radiation. After returning to a temperature in the vicinity of 20° C., the reaction medium is evaporated to dryness by evaporation under reduced pressure and the solid isolated is chromatographed, under argon pressure, on silica gel (eluent dichloromethane/methanol 95/5). 112 mg of N-benzylimidazo[1,2-a]pyrimidin-6-amine are thus obtained in the form of an orange lacquer.

MS: method A; [M+H]⁺ m/z=225; Tr=0.38 min.

### EXAMPLE 27

### N-[6-(imidazo[1,2-a]pyrimidin-3-ylsulphanyl)-1,3-benzothiazol-2-yl]tetrahydro-2H-pyran-4-carboxamide

The compound can be prepared as in Example 8 but starting from 350 mg of 6-(imidazo[1,2-a]pyrimidin-3-ylsulphanyl)-1,3-benzothiazol-2-amine, 1.5 g of tetrahydro-2H-pyran-4-carboxylic acid (commercially available product), 2.24 g of 1-(3-dimethylaminopropyl)-3-ethylcarbodiimide hydrochloride and 20 ml of anhydrous pyridine. 200 mg of N-[6-(imidazo[1,2-a]pyrimidin-3-ylsulphanyl)-1,3-benzothiazol-2-yl]tetrahydro-2H-pyran-4-carboxamide are thus obtained in the form of a white solid.

Melting point=270° C. (Köfler bench).

MS: method A; [M+H]⁺ m/z=412; [M−H]⁻ m/z=410; Tr=0.66 min.

¹H NMR (400 MHz, d₆-DMSO) δ ppm 1.54 to 1.82 (m, 4H); 2.67 to 2.87 (m, 1H); 3.35 (partially masked m, 2H); 3.90 (m, 2H); 7.14 to 7.27 (m, 2H); 7.63 (m, 1H); 7.85 (s, 1H); 8.24 (s, 1H); 8.69 (m, 1H); 8.87 (m, 1H); 12.34 (broad unresolved m, 1H).

### EXAMPLE 28

### Pharmaceutical Composition

Tablets corresponding to the following formulation were prepared:

| | |
|---|---|
| Product of Example 1 | 0.2 g |
| Excipient for a tablet made up to | 1 g |
| (particulars of the excipient: lactose, | |
| talc, starch, magnesium stearate). | |

Example 1 is taken as an example of a pharmaceutical preparation, it being possible for this preparation to be produced, if desired, with other products in the examples in the present patent application.

Pharmacological Part:

Experimental Protocols

I) Expression and Purification of MET, Cytoplasmic Domain

Expression in Baculovirus:

The His-Tev-MET (956-1390) recombinant DNA in pFastBac (Invitrogen) is transfected into insect cells and, after several viral amplification stages, the final baculovirus stock is tested for the expression of the protein of interest.

After infection for 72 h at 27° C. with the recombinant virus, the SF21 cell cultures are harvested by centrifugation and the cell pellets are stored at −80° C.

Purification:

The cell pellets are resuspended in lysis buffer (buffer A [50 mM HEPES, pH 7.5, 250 mM NaCl, 10% glycerol, 1 mM TECP]; + cocktail of protease inhibitors, Roche Diagnostics, without EDTA, ref. 1873580), stirred at 4° C. until the mixture is homogeneous and then lysed mechanically using a “Dounce” type apparatus.

After centrifugation, the lysis supernatant is incubated for 2 h at 4° C. with nickel chelate resin (His-Trap 6 Fast Flow™, GE HealthCare). After washing with 20 volumes of buffer A, the suspension is packed into a column, and the proteins are eluted with a gradient of buffer B (buffer A+290 mM imidazole).

The fractions comprising the protein of interest for the purpose of electrophoretic analysis (SDS PAGE) are combined, concentrated by ultrafiltration (10 kDa cut-off) and injected onto an exclusion chromatography column (Superdex™ 200, GE HealthCare) equilibrated in buffer A.

After enzymatic cleavage of the histidine tag, the protein is reinjected onto a new IMAC nickel chelate chromatography column (His-Trap 6 Fast Flow™, GE HealthCare) equilibrated in buffer A. The fractions eluted with a gradient of buffer B and comprising the protein of interest after electrophoresis (SDS PAGE) are finally combined and conserved at −80° C.

For the production of autophosphorylated protein, the previous fractions are incubated for 1 h at ambient temperature after the addition of 2 mM ATP, 2 mM MgCl₂, and 4 mM Na₃VO₄. After the reaction has been stopped with 5 mM of EDTA, the reaction mixture is injected onto a HiPrep desalifying column (GE HealthCare) preequilibrated in buffer A+4 mM Na₃VO₄, and the fractions comprising the protein of interest (SDS PAGE analysis) are combined and stored at −80° C. The degree of phosphorylation is verified by mass spectrometry (LC-MS) and by peptide mapping.

II) Tests A and B

A) Test A: HTRF MET Assay in 96-Well Format

MET at a final concentration of 5 nM is incubated in a final volume of 50 μl of enzymatic reaction in the presence of the test molecule (for a final concentration range of from 0.17 nM to 10 μM, 3% DMSO final concentration) in 10 mM MOPS buffer, pH 7.4, 1 mM DTT, 0.01% Tween 20. The reaction is initiated with the substrate solution to obtain final concentrations of 1 μg/ml poly-(GAT), 10 μM ATP and 5 mM MgCl₂. After incubation for 10 min at ambient temperature, the reaction is stopped with a 30 μl mix so as to obtain a final solution of 50 mM Hepes, pH 7.5, 500 mM potassium fluoride, 0.1% BSA and 133 mM EDTA in the presence of 80 ng of streptavidin 61SAXLB Cis-Bio Int. and 18 ng of anti-phosphotyrosine Mab PT66-Europium Cryptate per well. After incubation for 2 hours at ambient temperature, the reading is taken at 2 wavelengths, 620 nm and 665 nm, on a reader for the TRACE/HTRF technique and the % inhibition is calculated from the 665/620 ratios.

The results obtained with this test A for the products of formula (I) in examples in the experimental part are such that IC₅₀ is less than 500 nM and in particular less than 100 nM.

B) Test B: Inhibition of the Autophosphorylation of MET; ELISA Technique (pppY1230, 1234, 1235)

a) Cell lysates: Seed MKN45 cells into 96-well plates (Cell coat BD polylysine) at 20 000 cells/well in 200 μl in RPMI medium+10% FCS+1% L-glutamine. Leave to adhere for 24 hours in an incubator.

The cells are treated the day after seeding with the products at 6 concentrations in duplicate for 1 h. At least 3 control wells are treated with the same final amount of DMSO.

Product dilution: Stock at 10 mM in pure DMSO—range from 10 mM to 30 μM with an increment of 3 in pure DMSO—intermediate 1/50 dilutions in culture medium and then removal of 10 μl added directly to the cells (200 μl): final range from 10 000 to 30 nM.

At the end of the incubation, carefully remove the supernatant and rinse with 200 μl of PBS. Next, place 100 μl of lysis buffer directly in the wells on ice and incubate at 4° C. for 30 minutes. Lysis buffer: 10 mM Tris HCl, pH 7.4, 100 mM NaCl, 1 mM EDTA, 1 mM EGTA, 1% Triton X-100, 10% glycerol, 0.1% SDS, 0.5% deoxycholate, 20 mM NaF, 2 mM Na₃VO₄, 1 mM PMSF and cocktail of antiproteases.

The 100 μl of lysates are transferred into a V-bottomed polypropylene plate and the ELISA is performed immediately, or the plate is frozen at −80° C.

b) PhosphoMET ELISA BioSource Kit KH00281

Into each well of the kit plate, add 70 μl of kit dilution buffer+30 μl of cell lysate or 30 μl of lysis buffer for the blanks. Incubate for 2 h with gentle agitation at ambient temperature.

Rinse the wells 4 times with 400 μl of kit washing buffer. Incubate with 100 μl of anti-phospho MET antibody for 1 hour at ambient temperature.

Rinse the wells 4 times with 400 μl of kit washing buffer. Incubate with 100 μl of anti-rabbit HRP antibody for 30 minutes at ambient temperature (except for the wells of chromogen alone).

Rinse the wells 4 times with 400 μl of kit washing buffer. Introduce 100 μl of chromogen and incubate for 30 minutes in the dark at ambient temperature.

Stop the reaction with 100 μl of stop solution. Read without delay at 450 nM, 0.1 second on Wallac Victor plate reader.

C) Test C: Measurement of cell proliferation by ¹⁴C-thymidine pulse

The cells are seeded into Cytostar 96-well plates in 180 μl for 4 hours at 37° C. and 5% CO₂: HCT116 cells at a rate of 2500 cells per well in DMEM medium+10% foetal calf serum+1% L-glutamine and MKN45 cells at a rate of 7500 cells per well in RPMI medium+10% foetal calf serum+1% L-glutamine. After these 4 hours of incubation, the products are added in 10 μl as a 20-fold concentrated solution according to the dilution method mentioned for the ELISA. The products are tested at 10 concentrations in duplicate from 10 000 nM to 0.3 nM with an increment of 3.

After treatment for 72 h, add 10 μl of ¹⁴C-thymidine at 10 μCi/ml to obtain 0.1 μCi per well. The ¹⁴C-thymidine incorporation is measured on a Micro-Beta machine (Perkin-Elmer) after 24 hours of pulse and 96 h of treatment.

All the stages of the assay are automated on BIOMEK 2000 or TECAN stations.

The results obtained with this test B for the products of formula (I) in the examples in the experimental part are such that IC₅₀ is less than 10 microM and in particular less than 1 microM.

The results obtained for the products in the examples in the experimental part are given in the table of pharmacological results below, as follows:

for test A, the sign+corresponds to less than 500 nM and the sign++corresponds to less than 100 nM;

for test B, the sign+corresponds to less than 500 nM and the sign++corresponds to less than 100 nM;

for test C, the sign+corresponds to less than 10 microM and the sign++corresponds to less than 1 microM.

Table of pharmacological results:

| Example | Test A | Test B | Test C |
|---|---|---|---|
| 1 | ++ | + | ++ |
| 2 | ++ | ++ | ++ |
| 3 | ++ | ++ | ++ |
| 4 | ++ | ++ | ++ |
| 5 | ++ | ++ | ++ |
| 6 | + | | ++ |
| 7 | ++ | ++ | ++ |
| 8 | ++ | ++ | ++ |
| 9 | + | | ++ |
| 10 | ++ | ++ | ++ |
| 11 | ++ | + | ++ |
| 12 | + | | ++ |
| 13 | ++ | ++ | ++ |
| 14 | ++ | ++ | ++ |
| 15 | ++ | ++ | ++ |
| 16 | ++ | ++ | ++ |
| 17 | ++ | + | ++ |
| 18 | ++ | ++ | ++ |
| 19 | ++ | ++ | ++ |
| 20 | ++ | ++ | ++ |
| 21 | ++ | ++ | ++ |
| 22 | ++ | ++ | ++ |
| 23 | ++ | ++ | ++ |
| 24 | ++ | ++ | ++ |
| 25 | ++ | ++ | ++ |
| 26 | ++ | ++ | ++ |
| 27 | ++ | + | ++ |

We claim:

1) A product of formula (I): 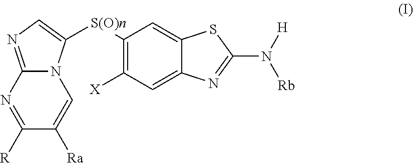
in which:
n=0, 1 or 2;
X represents a hydrogen atom, a halogen atom or an alkyl radical;
R represents a hydrogen atom or an NH₂, NHalk or N(alk)₂ radical;
Ra represents a hydrogen atom, a halogen atom or an —O-cycloalkyl, —O-alkyl, —O-aryl, —O-heteroaryl, —NRd(cycloalkyl), —NRd(alkyl), —NRd(aryl), —NRd(heteroaryl), alkyl, cycloalkyl, heterocycloalkyl, aryl or heteroaryl radical; the cycloalkyl, alkyl, aryl and heteroaryl radicals being, in all these radicals, optionally substituted as indicated below;
Rb represents a hydrogen atom or an Rc, —COORc, —CO—Rc or —CO—NRcRd radical;
with Rc representing an alkyl, cycloalkyl, heterocycloalkyl, aryl or heteroaryl radical, all these radicals being optionally substituted as indicated below;
Rd represents a hydrogen atom or an alkyl or cycloalkyl radical;
all the radicals defined above, alkyl, cycloalkyl, heterocycloalkyl, aryl and heteroaryl, being optionally substituted by one or more radicals chosen from halogen atoms and hydroxyl, alkoxyl, CN, CF₃, —NR1R2, —COOH, —COOalk, —CONR1R2, —NR1COR2, COR1, oxo, heterocycloalkyl, aryl and heteroaryl radicals, the latter heterocycloalkyl, aryl or heteroaryl being themselves optionally substituted by one or more radicals chosen from halogen atoms and hydroxyl, alkoxy, alkyl, CN, CF₃, —NR3R4, —COOH, —COOalk, —CONR3R4, —NR3COR4, —COR3 and oxo radicals;
the cycloalkyl, heterocycloalkyl, aryl or heteroaryl radicals being in addition optionally substituted by an alkyl radical itself optionally substituted by one or more radicals chosen from halogen atoms and hydroxyl, alkoxy, alkyl, NR3R4, —COOH, —COOalk, —CONR3R4, —NR3COR4 and —COR3 radicals;
NR1R2 being such that: either, R1 and R2 being identical or different, one of R1 and R2 represents a hydrogen atom or an alkyl radical and the other of R1 and R2 represents a hydrogen atom, a —CO₂-alkyl radical, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 3 to 10 ring members and optionally one or more other heteroatoms chosen from O, S and NH, this radical, including the optional NH which it comprises, being optionally substituted;
NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R3 and R4 form, with the nitrogen atom to which they are bonded, a cyclic radical including from 3 to 10 ring members and optionally one or more other heteroatoms chosen from O, S and NH, this radical, including the optional NH which it comprises, being optionally substituted;
the cyclic radicals which R1 and R2 or R3 and R4 respectively can form with the nitrogen atom to which they are bonded being optionally substituted by one or more identical or different radicals chosen from halogen atoms, hydroxyl, oxo, alkoxy, NH₂, NHalk and N(alk)₂ radicals and alkyl, cycloalkyl, heterocycloalkyl, —CO-alkyl, —CO₂Alk, phenyl, CH₂-phenyl and heteroaryl radicals, such that, in the latter radicals, the alkyl, heterocycloalkyl, phenyl and heteroaryl radicals are themselves optionally substituted by one or more radicals chosen from halogen atoms, hydroxyl radicals, alkyl and alkoxy radicals including from 1 to 4 carbon atoms, and NH₂, NHalk and N(alk)₂ radicals;
R1, R2, R3 and R4 in the —NR1COR2, —COR1, —NR3COR4 and —COR3 radicals being chosen from the meanings indicated above for R1, R2, R3 and R4 in NR1R2 and NR3R4 when R1 and R2, on the one hand, and R3 and R4 do not form a cyclic radical with the nitrogen atom to which they are bonded; and
all the alkyl(alk) and alkoxy radicals above include from 1 to 6 carbon atoms; or
an isomer or pharmaceutically acceptable salt thereof.

2) The product of claim 1, in which:
n=0, 1 or 2;
X represents a hydrogen atom, a fluorine atom or a methyl radical;
R represents a hydrogen atom or an NH₂ radical;
Ra represents a hydrogen atom, a halogen atom or an —O-cycloalkyl, —O-alkyl, —NRd(cycloalkyl), —NRd(alkyl), aryl or heteroaryl radical; in all these radicals, the cycloalkyl, alkyl, aryl and heteroaryl radicals being optionally substituted as indicated below;
Rb represents a hydrogen atom, a —CO—Rc radical or a —CO—NRcRd radical;
with Rc representing an alkyl, cycloalkyl, heterocycloalkyl or aryl radical, all optionally substituted by one or more radicals chosen from halogen atoms, hydroxyl, alkoxy and NR1R2 radicals and alkyl, heterocycloalkyl, aryl and heteroaryl radicals, themselves optionally substituted as indicated below;
Rd represents a hydrogen atom or an alkyl radical;
all the radicals defined above, alkyl, cycloalkyl, heterocycloalkyl, aryl and heteroaryl, being optionally substituted by one or more radicals chosen from halogen atoms and hydroxyl, alkoxy, —NR1R2, —COOH, —COOalk, —CONR1R2, alkyl and heterocycloalkyl radicals, itself optionally substituted by one or more radicals chosen from halogen atoms and hydroxyl, alkoxy, alkyl, —COOH, —COOalk, —NR3R4 and —CONR3R4 radicals;
NR1R2 being such that: either, R1 and R2 being identical or different, one of R1 and R2 represents a hydrogen atom or an alkyl radical and the other of R1 and R2 represents a hydrogen atom, a —CO₂-alk radical, a cycloalkyl radical or an alkyl radical optionally substituted by one or more identical or different radicals chosen from hydroxyl, alkoxy, NR3R4, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 3 to 10 ring members and optionally one or more other heteroatoms chosen from O, S and NH, this radical, including the optional NH which it comprises, being optionally substituted;
NR3R4 being such that: either, R3 and R4 being identical or different, one of R3 and R4 represents a hydrogen atom or an alkyl radical and the other of R3 and R4 represents a hydrogen atom, a cycloalkyl radical or an alkyl radical optionally substituted by one or more identical or different radicals chosen from hydroxyl, alkoxy, heterocycloalkyl, heteroaryl or phenyl radicals, themselves optionally substituted by one or more radicals chosen from halogen atoms and hydroxyl, alkyl, alkoxy, NH₂, NHalk and N(alk)₂ radicals; or R3 and R4 form, with the nitrogen atom to which they are bonded, a cyclic radical including from 3 to 10 ring members and optionally one or more other heteroatoms chosen from O, S and NH, this radical, including the optional NH which it comprises, being optionally substituted;
the cyclic radicals which R1 and R2 or R3 and R4 respectively can form with the nitrogen atom to which they are bonded being optionally substituted by one or more identical or different radicals chosen from halogen atoms, hydroxyl, oxo, alkoxy, NH₂, NHalk or N(alk)₂ radicals and alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and hydroxyl, alkyl, alkoxy, NH₂, NHalk and N(alk)₂ radicals; and
all the alkyl(alk) or alkoxy radicals above include from 1 to 6 carbon atoms; or
or an isomer or pharmaceutically acceptable salt thereof.

3) The product according to claim 1, in which:
n=0, 1 or 2;
X represents a hydrogen atom or a fluorine atom;
R represents a hydrogen atom or an NH₂ radical;
Ra represents a hydrogen atom, a halogen atom, an —O-cycloalkyl radical, an —NH-cycloalkyl radical, an —NH-alk-phenyl radical or a phenyl radical, all these cycloalkyl and phenyl radicals being optionally substituted by one or more radicals chosen from halogen atoms and hydroxyl, alkoxy, —NR1R2, —COOH, —COOalk, —CONR1R2, alkyl and heterocycloalkyl radicals, themselves optionally substituted by one or more radicals chosen from halogen atoms and alkyl, —COOH, —COOalk and —CONR3R4 radicals;
Rb represents a hydrogen atom, a —CO—Rc radical or a —CO—NRcRd radical;
with Rc representing an alkyl, cycloalkyl, heterocycloalkyl or aryl radical, all optionally substituted by one or more radicals chosen from hydroxyl, alkoxy, NR1R2, alkyl, heterocycloalkyl and phenyl radicals, the latter alkyl, heterocycloalkyl and phenyl radicals being themselves optionally substituted by one or more radicals chosen from halogen atoms and hydroxyl, alkoxy, alkyl and NR3R4 radicals;
Rd represents a hydrogen atom or an alkyl radical;
NR1R2 being such that either, R1 and R2 being identical or different, one of R1 and R2 represents a hydrogen atom or an alkyl radical and the other of R1 and R2 represents a hydrogen atom, a cycloalkyl radical, a CO₂alk radical or an alkyl radical optionally substituted by one or more identical or different radicals chosen from hydroxyl, alkoxy, NR3R4 or phenyl radicals, itself optionally substituted by one or more radicals chosen from halogen atoms and hydroxyl, alkyl, alkoxy, NH₂, NHalk and N(alk)₂ radicals; or R1 and R2 form, with the nitrogen atom to which they are bonded, a cyclic radical including from 4 to 7 ring members and optionally another heteroatom chosen from O, S and NH, this radical, including the optional NH which it comprises, being optionally substituted;
NR3R4 being such that either R3 and R4, which are identical or different, represent a hydrogen atom or an alkyl radical optionally substituted by one or more identical or different radicals chosen from hydroxyl or alkoxy radicals or R3 and R4 form, with the nitrogen atom to which they are bonded, a cyclic radical including from 4 to 7 ring members and optionally another heteroatom chosen from O, S and NH, this radical, including the optional NH which it comprises, being optionally substituted;
the cyclic radicals which R1 and R2 or R3 and R4 respectively can form with the nitrogen atom to which they are bonded being optionally substituted by one or more identical or different radicals; and
all the alkyl(alk) or alkoxy radicals above include from 1 to 4 carbon atoms; or
or an isomer or pharmaceutically acceptable salt thereof.

4) The product according to claim 1, in which:
n=0, 1 or 2;
X represents a hydrogen atom or a fluorine atom;
R represents a hydrogen atom or an NH₂ radical;
Ra represents a hydrogen atom, a halogen atom, an —O-cycloalkyl radical, an —NH-cycloalkyl radical, an —NH-alk-phenyl radical or a phenyl radical, the phenyl radicals being optionally substituted by one or more radicals chosen from halogen atoms and the alkyl radical;
Rb represents a hydrogen atom, a —CO—Rc radical or a —CO—NRcRd radical;
with Rc representing an alkyl, cycloalkyl, heterocycloalkyl or phenyl radical, all optionally substituted by one or more radicals chosen from hydroxyl, alkoxy, NR1R2, alkyl and heterocycloalkyl radicals, the latter alkyl and heterocycloalkyl radicals being themselves optionally substituted by one or more radicals chosen from halogen atoms and the hydroxyl, alkoxy, alkyl and NR3R4 radicals;
Rd represents a hydrogen atom;
NR1R2 being such that either R1 and R2, which are identical or different, represent a hydrogen atom or an alkyl radical optionally substituted by one or more identical or different radicals chosen from hydroxyl, alkoxy, NH₂, NHalk and N(alk)₂ radicals or else NR1R2 represents the —NHCO₂alk radical; or R1 and R2 form, with the nitrogen atom to which they are bonded, a cyclic radical including from 4 to 7 ring members and optionally another heteroatom chosen from O, S and NH, optionally substituted by one or more identical or different radicals chosen from oxo, NH₂, NHalk, N(alk)₂, alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and alkyl, hydroxyl, alkoxy, NH₂, NHalk and N(alk)₂ radicals;
NR3R4 being such that either R3 and R4, which are identical or different, represent a hydrogen atom or an alkyl radical optionally substituted by one or more identical or different radicals chosen from hydroxyl or alkoxy radicals or R3 and R4 form, with the nitrogen atom to which they are bonded, a cyclic radical including from 4 to 7 ring members and optionally another heteroatom chosen from O, S and NH, this radical, including the optional NH which it comprises, being optionally substituted by an alkyl or phenyl radical, themselves optionally substituted by one or more identical or different radicals chosen from halogen atoms and alkyl, hydroxyl, alkoxy, NH₂, NHalk and N(alk)₂ radicals; and
all the alkyl(alk) or alkoxy radicals above include from 1 to 4 carbon atoms; or
or an isomer or pharmaceutically acceptable salt thereof.

5) The product according to claim 1, in which:
n=0, 1 or 2;
X represents a hydrogen atom or a fluorine atom;
R represents a hydrogen atom or an NH₂ radical;
Ra represents a hydrogen atom, a —O-cycloalkyl radical, a —NH-cycloalkyl radical, a —NH-alk-phenyl radical or a phenyl radical, the phenyl radicals being optionally substituted by a halogen atom;
Rb represents a hydrogen atom, a —CO—Rc radical or a —CO—NRcRd radical;
with Rc representing a cycloalkyl radical optionally substituted by an alkyl radical itself optionally substituted by a morpholino radical; a heterocycloalkyl radical optionally substituted by an alkyl radical; a phenyl radical; or an alkyl radical substituted by an alkoxy, NR1R2 or heterocycloalkyl radical, itself optionally substituted by one or more radicals chosen from halogen atoms and alkyl radicals;
Rd represents a hydrogen atom;
NR1R2 being such that either R1 and R2, which are identical or different, represent a hydrogen atom or an alkyl radical or else NR1R2 represents the —NHCO₂alk radical; or R1 and R2 form, with the nitrogen atom to which they are bonded, a cyclic radical including from 4 to 7 ring members and optionally another heteroatom chosen from O, S, N and NH, optionally substituted by one or more identical or different radicals chosen from oxo, NH₂, NHalk and N(alk)₂ radicals and alkyl, cycloalkyl, heterocycloalkyl, —CO-alkyl, —CO₂alk, phenyl and CH₂-phenyl radicals, in which the alkyl, heterocycloalkyl and phenyl radicals are themselves optionally substituted by one or more identical or different radicals chosen from halogen atoms and alkyl, hydroxyl, alkoxy, NH₂, NHalk and N(alk)₂ radicals; and
the alkyl or alkoxy radicals above include from 1 to 4 carbon atoms; or
or an isomer or pharmaceutically acceptable salt thereof.

6) The product according to claim 1, wherein the product is:
6-(imidazo[1,2-a]pyrimidin-3-ylsulphanyl)-1,3-benzothiazol-2-amine;
N-[6-(imidazo[1,2-a]pyrimidin-3-ylsulphanyl)-1,3-benzothiazol-2-yl]cyclopropanecarboxamide;
N-[6-(imidazo[1,2-a]pyrimidin-3-ylsulphanyl)-1,3-benzothiazol-2-yl]acetamide;
1-[6-(imidazo[1,2-a]pyrimidin-3-ylsulphanyl)-1,3-benzothiazol-2-yl]-3-[2-(morpholin-4-yl)ethyl]urea;
1-[6-(imidazo[1,2-a]pyrimidin-3-ylsulphanyl)-1,3-benzothiazol-2-yl]-3-[2-(pyrrolidin-1-yl)ethyl]urea;
N-[6-(imidazo[1,2-a]pyrimidin-3-ylsulphinyl)-1,3-benzothiazol-2-yl]cyclopropanecarboxamide;
N-[6-(imidazo[1,2-a]pyrimidin-3-ylsulphonyl)-1,3-benzothiazol-2-yl]cyclopropanecarboxamide;
N-[6-(imidazo[1,2-a]pyrimidin-3-ylsulphanyl)-1,3-benzothiazol-2-yl]-3-(pyrrolidin-1-yl)propanamide;
N-[6-(imidazo[1,2-a]pyrimidin-3-ylsulphanyl)-1,3-benzothiazol-2-yl]benzamide;
N-[6-(imidazo[1,2-a]pyrimidin-3-ylsulphanyl)-1,3-benzothiazol-2-yl]-2-(4-methylpiperazin-1-yl)acetamide;
2-methylpropan-2-yl(2-{[6-(imidazo[1,2-a]pyrimidin-3-ylsulphanyl)-1,3-benzothiazol-2-yl]amino}-2-oxoethyl)carbamate;
N-[6-(imidazo[1,2-a]pyrimidin-3-ylsulphanyl)-1,3-benzothiazol-2-yl]glycinamide dihydrochloride;
(trans-A)-N-[6-(imidazo[1,2-a]pyrimidin-3-ylsulphanyl)-1,3-benzothiazol-2-yl]-2-(morpholin-4-ylmethyl)cyclopropanecarboxamide;
(trans-B)-N-[6-(imidazo[1,2-a]pyrimidin-3-ylsulphanyl)-1,3-benzothiazol-2-yl]-2-(morpholin-4-ylmethyl)cyclopropanecarboxamide;
2-(4-ethylpiperazin-1-yl)-N-[6-(imidazo[1,2-a]pyrimidin-3-ylsulphanyl)-1,3-benzothiazol-2-yl]acetamide;
2-(4-cyclopropylpiperazin-1-yl)-N-[6-(imidazo[1,2-a]pyrimidin-3-ylsulphanyl)-1,3-benzothiazol-2-yl]acetamide;
N₂,N₂-diethyl-N-[6-(imidazo[1,2-a]pyrimidin-3-ylsulphanyl)-1,3-benzothiazol-2-yl]glycinamide;
N-[5-fluoro-6-(imidazo[1,2-a]pyrimidin-3-ylsulphanyl)-1,3-benzothiazol-2-yl]cyclopropanecarboxamide;
5-fluoro-6-(imidazo[1,2-a]pyrimidin-3-ylsulphanyl)-1,3-benzothiazol-2-amine;
N-[6-(imidazo[1,2-a]pyrimidin-3-ylsulphanyl)-1,3-benzothiazol-2-yl]-3-methoxypropanamide;
N-[6-(imidazo[1,2-a]pyrimidin-3-ylsulphanyl)-1,3-benzothiazol-2-yl]-2-(4-methyl-3-oxopiperazin-1-yl)acetamide;
N-{6-[(7-aminoimidazo[1,2-a]pyrimidin-3-yl)sulphanyl]-1,3-benzothiazol-2-yl]cyclopropanecarboxamide;
N-(6-{[6-(3-fluorophenyl)imidazo[1,2-a]pyrimidin-3-yl]sulphanyl}-1,3-benzothiazol-2-yl)cyclopropanecarboxamide;
N-(6-{[6-(cyclohexyloxy)imidazo[1,2-a]pyrimidin-3-yl]sulphanyl}-1,3-benzothiazol-2-yl)cyclopropanecarboxamide;
3-[(2-amino-1,3-benzothiazol-6-yl)sulphanyl]-N-cyclohexylimidazo[1,2-a]pyrimidin-6-amine;
N-(6-{[6-(benzylamino)imidazo[1,2-a]pyrimidin-3-yl]sulphanyl}-1,3-benzothiazol-2-yl)cyclopropanecarboxamide;
N-[6-(imidazo[1,2-a]pyrimidin-3-ylsulphanyl)-1,3-benzothiazol-2-yl]tetrahydro-2H-pyran-4-carboxamide; or
a pharmaceutically acceptable salt thereof.

7) A process for preparing a product according to claim 1, said process comprising the following steps: 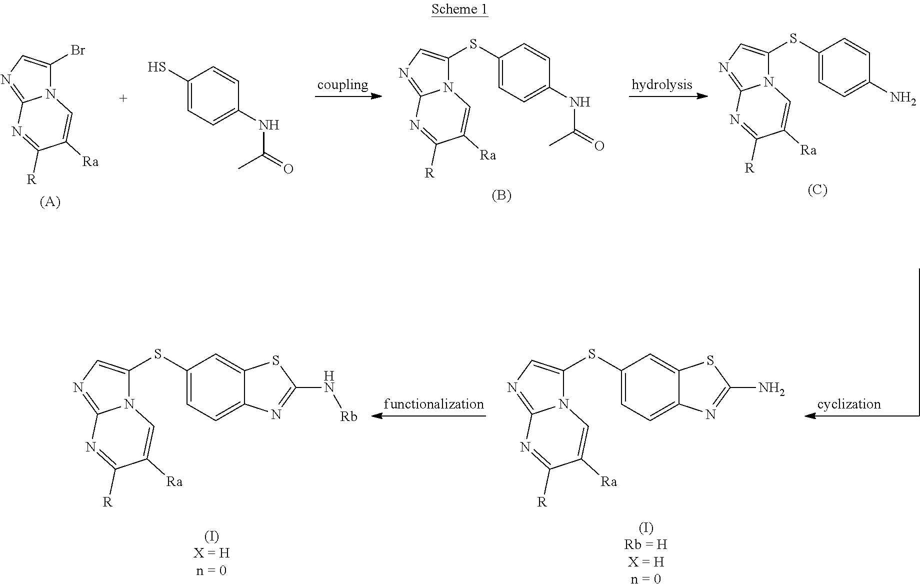
in which the Ra, Rb and R substituents have the meanings indicated in claim 1, X═H and n=0.

8) A process for preparing a product according to claim 1, said process comprising the following steps: 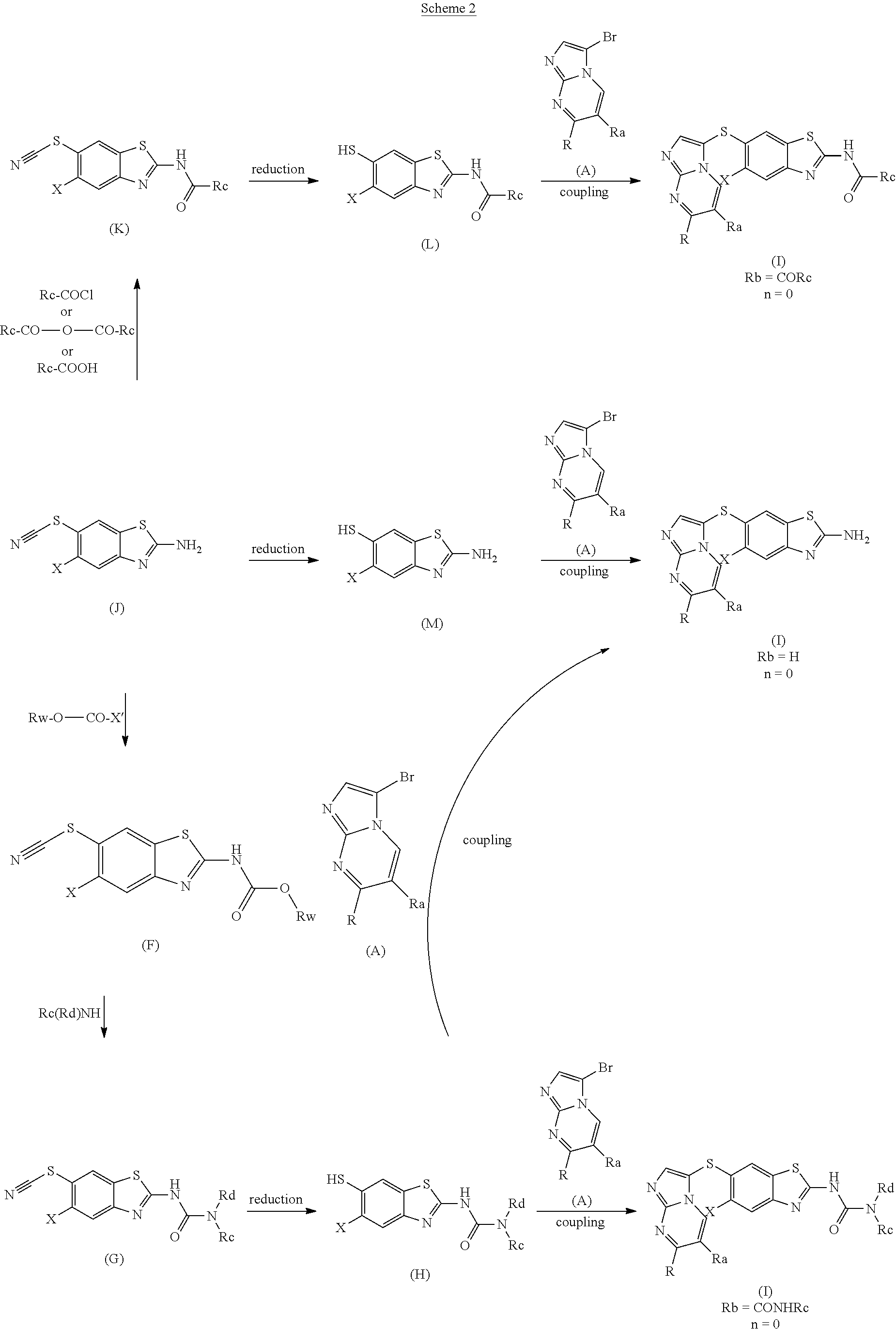
in which Rw represents a phenyl radical and the Ra, Rc, Rd, R and X substituents have the meanings indicated in claim 1 and n=0.

9) A process for preparing a product according to claim 1, said process comprising the following steps: 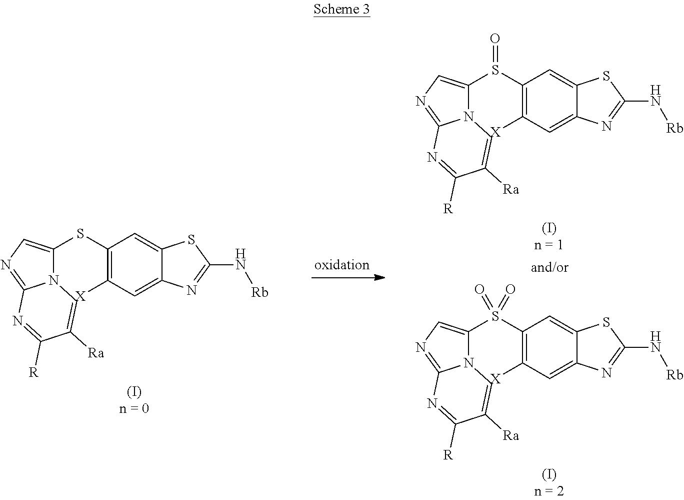
in which the Ra, Rb, R and X substituents have the meanings indicated in claim 1.

10) A pharmaceutical composition comprising a product of claim 1.

11) A pharmaceutical composition comprising a product of claim 6.

12) A pharmaceutical composition comprising at least one product according to claim 1, or a prodrug thereof, and a pharmaceutically acceptable carrier.

13) A method of inhibiting the activity of MET protein kinase in a patient in need thereof comprising administering to said patient a product according to claim 1.

14) A method of treating or preventing a disease in a patient in need thereof comprising administering to said patient a product according to claim 1, wherein said disease is chosen from the group consisting of blood vessel proliferation disorders, fibrotic disorders, “mesangial” cell proliferation disorders, metabolic disorders, allergies, asthma, thrombosis, diseases of the nervous system, retinopathy, psoriasis, rheumatoid arthritis, diabetes, muscle degeneration and cancers.

15) A method of treating cancer in a patient in need thereof comprising administering to said patient a product according to claim 1.

16) The method according to claim 15, wherein said cancer is in the form of a solid or liquid tumour.

17) The method according to claim 15, wherein said cancer has shown resistance to other cytotoxic agents.

18) The method according to claim 15, wherein the cancer is gastric cancer, liver cancer, kidney cancer, ovarian cancer, colon cancer, prostate cancer, lung cancer (NSCLC or SCLC), glioblastoma, thyroid cancer, bladder cancer, breast cancer, melanoma, lymphoid or myeloid haematopoietic tumour, sarcoma, brain cancer, laryngeal cancer, lymphatic cancer, bone cancer or pancreatic cancer.

19) The product according to claim 1, wherein said product is a kinase inhibitor.

20) The product according to claim 1, wherein said product is a MET inhibitor.

21) A product having one of the following formulas: 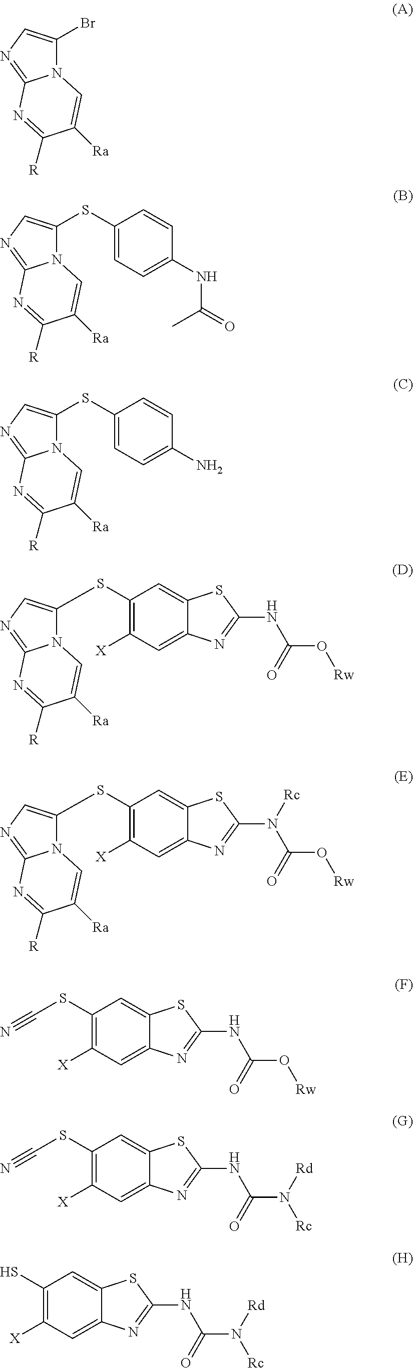 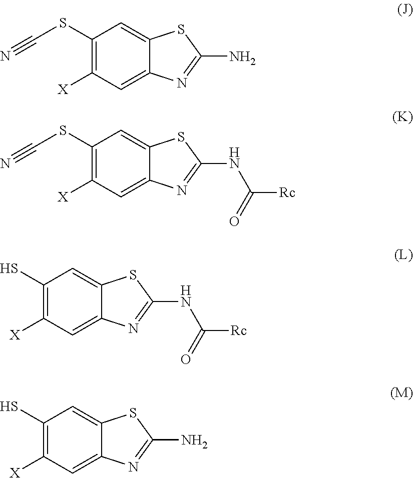
in which Ra, Rb, Rc, Rd, R and X have the definitions indicated in claim 1 and Rw represents a t-butyl or phenyl radical.
